(19) United States
(12) Patent Application Publication
(10) Pub. No.: US 2002/0055628 A1
(43) Pub. Date: May 9, 2002

(54) Multilocus repetitive DNA sequences for genotyping bacillus anthracis and related bacteria

(76) Inventors: Paul S. Keim, Flagstaff, AZ (US); Paul J. Jackson, Los Alamos, NM (US)

Correspondence Address: Samuel M. Freund, Los Alamos National Laboratory, LC/BPL, MS D412, Los Alamos, NM, 87545, US

(21) Appl. No.: 09/842,552

(22) Filed: Apr. 26, 2001

Related U.S. Application Data
(60) Provisional application No. 60/199,911, filed on Apr. 26, 2000.

(51) Int. Cl. C07H 21/04

(52) U.S. Cl. 536/23.7

(57) ABSTRACT

Bacillus anthracis is one the most molecularly homogeneous pathogens described, which makes strain discrimination particularly difficult. The present invention includes a molecular-typing method based upon rapidly evolving variable number tandem repeat (VNTR) loci. Multiple-locus VNTR analysis (MLVA) combines the information from multiple alleles at several marker loci. PCR amplification products from eight VNTR regions are detected and sized using fluorescently labeled primers. Five of these eight loci were discovered by characterization of AFLP markers (vrrC₁, vrrC₂, vrrB₁, vrrB₂ and CG3); two were discovered from complete plasmid nucleotide sequences (pXO1-aat, pXO2-at); and, one was previously known (vrrA). 425 isolates were characterized to identify 89 distinct genotypes. VNTR markers frequently had multiple alleles (from 2 to 8) and diversity (D) values between 0.3 and 0.8. UPGMA cluster analysis identified six genetically distinct groups that appear to represent genetic clones. Some of these clones show worldwide distribution, while others are restricted to particular geographic regions. The present method is also applicable to related bacteria. An additional 28 loci having variable repeat units have been identified by examining the B. anthracis DNA sequence, but these loci have not yet been utilized in the identification of B. anthracis strains.

### RELATED CASES

The present non-provisional patent application claims the benefit of Provisional Application Serial No. 60/199,911 which was filed on Apr. 26, 2000.

### STATEMENT REGARDING FEDERAL RIGHTS

This invention was made with government support under Contract No. W-7405-ENG-36 awarded by the U. S. Department of Energy to The Regents of The University of California. The government has certain rights in the invention.

### FIELD OF THE INVENTION

The present invention relates generally to molecular typing of Bacillus anthracis and, more particularly, to the utilization of variable number tandem repeat (VNTR) loci for the identification of genotypes of Bacillus anthracis and related bacteria.

### BACKGROUND OF THE INVENTION

Anthrax is a disease that has plagued mankind for millennia. While anthrax currently affects mostly livestock and wildlife around the world, it can and does kill humans. Current interest in anthrax is related to its potential as a bioterrorism agent with devastating impact; the spores of Bacillus anthracis can remain stable for scores of years and can be readily packaged into biological weapons. This same longevity may greatly influence the ecology and evolution of this pathogen. The initiating spores for an anthrax outbreak may emanate from a single long-deceased victim. Dormancy reduces the rate of evolutionary change and may contribute to the extremely homogeneous nature of B. anthracis.

Numerous studies have encountered the lack of molecular polymorphism within B. anthracis (See, e.g., L. J. Harrell et al., “Genetic variability of Bacillus anthracis and related species” J. Clin. Microbiol. 33:1847-1850 (1995), I. Henderson et al., “Differentiation of Bacillus anthracis and other ‘Bacillus cereus group’ bacteria using IS231-derived sequences” FEMS Microbiol. Lett. 128:113-118 (1995), and P. Keim et al., “Molecular evolution and diversity in Bacillus anthracis as detected by amplified fragment length polymorphism markers” J. Bacteriol. 179:818-824 (1997)).

Previous analyses using amplified fragment length polymorphisms (AFLP) observed only 30 differences among >1000 fragments (See Keim et al., supra). Many of these AFLP markers had low diversity values and little discriminatory power. A comparative DNA sequencing study of the protective antigen gene found only five differences across 2500 nucleotides in 25 diverse strains (See, e.g., L. B. Price et al., “Natural Genetic Diversity in the Protective Antigen Gene of Bacillus anthracis” J. Bacteriol. 181:2358-2362 (1999)).

An exception to this trend may be found in G. L. Andersen et al., “Identification of a region of genetic variability among Bacillus anthracis strains and related species” J. Bacteriol. 178:377-384 (1996) in which a previously identified AP-PCR marker (See Henderson et al., supra) was examined by DNA sequence analysis. A large ORF (vrrA) that contained a variable number tandemly repeated (VNTR) sequence was found. By contrast with the extreme monomorphic nature of the genome, five different allelic states were observed in the vrrA VNTR among diverse strains (See, e.g., P. E. Jackson et al., “Characterization of the variable-number tandem repeats in vrrA from different Bacillus anthracis isolates” Appl. Environ. Microbiol. 63:1400-1405 (1997)). This demonstrated that even highly similar B. anthracis strains could be differentiated if diverse genomic regions could be identified. Such discrimination is essential if molecular epidemiology is to aid in the understanding and control of anthrax.

Molecular typing of pathogens has long been a part of disease control and has recently been accelerating with new technologies. Traditionally, serotyping has been extremely valuable and was often able to identify important cellular components associated with virulence. While serotyping will continue to be an important tool, it often has limited discriminatory power that resolves pathogens into only a few types. Multi-locus enzyme electrophoresis (MLEE) provides a multiple factor genetic analysis with as many as 40 genetic loci analyzed (See, e.g., E. F. Boyd et al., “Molecular genetic relationships of the salmonellae” Appl. Environ. Microbiol. 62, 804 (1996)). In addition, enzyme loci frequently have greater than two alleles providing increased genetic resolution per locus. However, DNA typing has an even greater capacity for genetic dissection of bacterial pathogens and is limited only by the genome size and the technology. With genome sizes being in the millions of nucleotides, technology is invariably limiting. Pulse-field gel electrophoresis (PFGE) can resolve very large DNA restriction fragments, which has proven generally applicable to many pathogens and has notable successes in the epidemiological tracking (See, e.g., M. K. Mieftinen et al., “Molecular epidemiology of an outbreak of febrile gastroenteritis caused by Listeria monocytogenes in cold-smoked rainbow trout” J. Clin. Microbiol. 37, 2358 (1999)). However, PFGE is a cumbersome technology, which cannot easily handle very large sample sets, nor are PFGE data sets easily standardized for transfer throughout the public health community. Ribotyping uses restriction fragment length polymorphisms associated with rRNA genes (See, e.g., T. C. Popovic et al., “Use of molecular subtyping to document long-term persistence of Corynebacterium diphtheriae in South Dakota” J. Clin. Microbiol. 37:1092-1099 (1999)) and is generally applicable to all bacteria but is limited by the number of ribosomal loci in the genome.

Recently, PCR-based methods have exploded onto the molecular typing effort. These approaches include AFLPs, REP-PCR, RADPs and AP-PCR (See, e.g., J. Welsh and M. McClelland “Fingerprinting genomes using PCR with arbitrary primers” Nucleic Acids Res. 18:7213-7218 (1990), P. R. Vos et al., “AFLP: a new technique for DNA Fingerprinting” Nucleic Acids Res. 23:4407-4414 (1995.), and

J. G. Williams et al., “DNA polymorphisms amplified by arbitrary primers are useful as genetic markers” Nucleic Acids Res. 18, 6531 (1990). The power of PCR-based methods is the ease in which they can be applied to many bacterial pathogens and their multi-locus discrimination. These methods have proven valuable for genetic dissection of pathogens where other approaches have failed. However, a limitation of many PCR-based approaches is the biallelic (binary) nature of their data; frequently the presence or absence of a marker fragment. Comparative gene sequencing is becoming feasible for strain characterization and can be performed at multiple loci. Multiple locus sequence typing (MLST) can provide multiple alleles (haplotypes) per locus at well-defined and genomically dispersed locations (See, Maiden et al., supra). Nucleotide data are already standardized and very useful for phylogenetic analyses. If sufficient nucleotide diversity is present, MLST can distinguish among species and strains. While routine clinical MLST is still unfeasible, hybridization arrays (e.g., chip technology) could make single nucleotide polymorphisms (SNPs) a main stream approach to pathogen typing in the future (See, e.g., M. Vahey et al., “Performance of the Affymetrix GeneChip HIV PRT 440 platform for antiretroviral drug resistance genotyping of human immunodeficiency virus type 1 clades and viral isolates with length polymorphisms” J. Clin. Microbiol. 37:2533-2537 (1999).).

One of the most recent developments in molecular typing involves variable number tandemly repeated (VNTR) sequences (See, e.g., A. van Belkum et al., “Short-sequence DNA repeats in prokaryotic genomes” Microbiol. Mol. Biol. Rev. 62:275-93 (1998)). Short nucleotide sequences that are repeated multiple times often vary in copy number, creating length differences that can be detected by PCR using flanking primers. VNTRs appear to contain greater diversity and, hence, greater discriminatory capacity than any other type of molecular typing system (See van Belkum et al., supra). Many bacteria have VNTRs, though development of the PCR primers for these markers is specific to each pathogen. P. Keim et al. in “Molecular diversity in Bacillus anthracis” J. Appl. Microbiol. 87: 215-217 (1999) have identified eight novel variable number tandemly repeated loci from previously known amplified fragment length polymorphism markers or from the DNA sequence for Bacillus anthracis. The variable number tandem repeat (VNTR) loci are found in both gene coding (genic) and non-coding (non-genic) regions.

Accordingly, it is an object of the present invention to provide a method for molecular typing of Bacillus anthracis.

Another object of the present invention is to utilize rapidly evolving variable number tandem repeat (VNTR) loci for molecular typing of Bacillus anthracis.

Additional objects, advantages and novel features of the invention will be set forth, in part, in the description that follows, and, in part, will become apparent to those skilled in the art upon examination of the following or may be learned by practice of the invention. The objects and advantages of the invention may be realized and attained by means of the instrumentalities and combinations particularly pointed out in the appended claims.

### SUMMARY OF THE INVENTION

To achieve the foregoing and other objects of the present invention, and in accordance with its purposes as embodied and broadly described herein, the method for discriminating among different B. anthracis isolates hereof includes a multiple-locus VNTR analysis (MLVA) procedure using eight marker loci. Five of these markers (vrrC₁, vrrC₂, vrrB₁ ,vrrB₂ and CG3) were identified by the nucleotide sequence characterization of B. anthracis AFLP markers (See, “Molecular Evolution And Diversity In Bacillus anthracis As Detected By Amplified Fragment Length Polymorphism Markers” by Paul S. Keim et al., J. Bacteriol. 179, 818. (1997)). One marker (vrrA) was identified previously (See, “Identification Of A Region Of Genetic Variability Among Bacillus anthracis Strains And Related Species” by, G. L. Andersen et al., J. Bacteriol. 178, 377 (1996), and two were identified by analysis of the pXO1 and pXO2 plasmid sequences (pXO1-aat and pXO2-at) (See, “Molecular Epidemiology Of An Outbreak Of Febrile Gastroenteritis Caused By Listeria Monocytogenes In Cold-Smoked Rainbow Trout” by M. K. Miettinen et al., J. Clin. Microbiol. 37, 2358 (1999)). Because of the nearly monomorphic molecular nature of B. anthracis, MLVA may be the only reasonable method to study the diversity, evolution and molecular epidemiology of this pathogen. Analysis of a worldwide B. anthracis collection reveals 89 distinct MLVA genotypes that cluster into approximately six major genetic groups that represent worldwide clones.

Benefits and advantages of the present invention include a robust and easily transferable approach to characterizing B. anthracis isolates. The protocols presented are rapid and require only crudely isolated DNA to provide high-resolution molecular typing analysis. The individual marker alleles are uniquely identified by a combination of size and fluorescent color. Therefore, automated gel analysis is routine, and instrumentation for performing MLVA are widely available.

### BRIEF DESCRIPTION OF THE DRAWINGS

The accompanying drawings, which are incorporated in and form a part of the specification, illustrate the embodiments of the present invention and, together with the description, serve to explain the principles of the invention. In the drawings:

FIG. 1 is a fluorescent image of an ABI377 electrophoresis gel containing amplification products from the eight VNTR loci against 32 B. anthracis isolates, each marker allele having a unique size and color combination, thereby permitting exact identification.

FIG. 2 is a Dendrogram showing genotype results using the MLVA method of the present invention.

### DETAILED DESCRIPTION

Briefly, the method of the present invention includes a multiple-locus VNTR analysis (MLVA) system which uses the combined power of multiple alleles at several marker loci. Eight marker loci effectively discriminate among different isolates of B. anthracis. Five of these markers (vrrC₁, vrrC₂, vrrB₁, vrrB₂ and CG3) were identified through the nucleotide sequence characterization of B. anthracis AFLP markers (See Keim et al., supra). One was from a previous report (vrrA) (See Anderson et al., supra) and two were from the pXO1 and pXO2 plasmid sequences (pXO1-aat and pXO2-at) (See, R. Okinaka et al., “The Structure and Organization of pXO1, the toxin containing plasmid of Bacillus anthracis” J. Bacteriol. 181, 6509 (1999)). The variable AFLP DNA fragments were sequenced, flanking regions characterized, and locus-specific PCR primers developed. Likewise, locus-specific primers were designed around the variable plasmid regions. The locus-specific PCR primers were optimized for multiplexed analysis using primers from the previously described vrrA locus (See Andersen et al., supra). These loci also exist in the close relatives of B. anthracis such as B. thuringesis, B. mycoides, B. cereus, and have been shown to vary greatly in these species as well. Therefore, similar primers can be optimized for the analysis of those species.

TABLE 1 shows the primers used in DNA fingerprinting of B. anthracis.

TABLE 1

| Marker | | | Dye | Tm |
|---|---|---|---|---|
| Locus | Primer name | Primer sequence | label | ° C. |
| VrrA | VrrA-f1-fam | CAC AAC TAC CAC CGA TGG CAC A | fam | 71.0 |
| | (SEQ ID NO:1) | | | |
| | vrrA-r1 | GCG CGT TTC GTT TGA TTC ATA C | none | 69.7 |
| | (SEQ ID NO:2) | | | |
| VrrB₁ | VrrB₁-f1-fam | ATA GGT GGT TTT CCG CAA GTT ATT | fam | 70.0 |
| | (SEQ ID NO:3) | C | | |
| | vrrB₁-r1 | GAT GAG TTT GAT AAA GAA TAG CCT | none | 69.0 |
| | (SEQ ID NO:4) | GTG | | |
| VrrB₂ | VrrB₂-f1-fam | CAC AGG CTA TTC TTT ATC AAA CTC | fam | 72.0 |
| | (SEQ ID NO:5) | ATC | | |
| | vrrB₂-r1 | CCC AAG GTG AAG ATT GTT GTT GA | none | 68.8 |
| | (SEQ ID NO:6) | | | |
| VrrC₁ | VrrC₁-f1 | GAA GCA AGA AAG TGA TGT AGT GGA | none | 66.8 |
| | (SEQ ID NO:7) | C | | |
| | vrrC₁-r1-fam | CAT TTC CTC AAG TGC TAC AGG TTC | fam | 67.5 |
| | (SEQ ID NO:8) | | | |
| VrrC₂ | VrrC₂-f1-hex | CCA GAA GAA GTG GAA CCT GTA GCA | hex | 70.9 |
| | (SEQ ID NO:9) | C | | |
| | vrrC₂-r1 | GTC TTT CCA TTA ATC GCG CTC TAT | none | 70.6 |
| | (SEQ ID NO:10) | C | | |
| CG3 | CG3-f1-ned | TGT CGT TTT ACT TCT CTC TCC AAT | ned | 66.2 |
| | (SEQ ID NO:11) | AC | | |
| | CG₃₋ᵣ₁ | ACT CAT TGT TCT CTA TAA AGG GCA | none | 66.2 |
| | (SEQ ID NO:12) | T | | |
| pX01-aat | PX01-AAT-f3-fam | CAA TTT ATT AAC CAT CAG ATT AAG | fam | 66.3 |
| | (SEQ ID NO:13) | TTC A | | |
| | pX01-AAT-r3 | TCT ACA ATT ACT TCC TTC ATA ATC | none | 66.7 |
| | (SEQ ID NO:14) | CC | | |
| pX02-at | PX02-AT-f1-hex | TCA TCC TCT TTT AAC TCT TGG GT | hex | 64.4 |
| | (SEQ ID NO:15) | | | |
| | pX02-AT-r1 | GTG TGA TCA AGT CCG ACG ACA | none | 65.8 |
| | (SEQ ID NO:16) | | | |

TABLE 2 shows the DNA sequence from the variable regions of Bacillus anthracis, the sequence regions corresponding to the PCR primers listed in Table 1 are blocked.

TABLE 2

```
          a) The VrrB locus (SEQ ID NO:17):
          ACACAAACATCAAGAAAXAACAGAAGATATGCAGAGCTTCGCTTCAACA
CAGCGATTTCTCAAATGATGGTGTTCATCAACGATGCATACAAAGCTGAAACAC
TTCCGAAAGAATATGTAGAAGGTTTCGTAAAAATGATTGCACCAGTTGCACCTC
ACATCGGGGAAGAGCTATGGAGCAAACTTGGATACAATGAAACAATCACATAT
GCAAGCTGGCCAACATTTGATGAGTCTAAACTTGTAGAAGATGAAGTTGAAATC
GTTGTTCAAGTTATGGGTAAAGTTCGCGCAAAACTAACAATGAGTAAAGACGCA
TCAAAAGACGAAATGGAAAAACTTGCACTTGAAGCAATTCAAGACCAAATCGA
AGGAAAAACAGTTCGTAAAGTAATTGTAGTTCCTGGAAAACTTGTTAACGTTGT
TGCAAACTAATTCATTCTAAATAAAAGCTCAGAGTATTTATGCTCTGAGCTTTTT
TGTGTAGAAAATAATTTCTGGATCAGCTTCATCTAAATTTTCAACTATTCCACTA
CGTATAAAACCGTTCGCGTTAAATACTTTCTGCATACTTTCATTCGATTCATTCG
TTGAAGAAAAGAAGCGGAATTCCAATACAGACAAACACCTATTTTTTTATATGA
AATATGATAAAAAAGGTAGGCTAAATTGAGTAGAAGAGGTGAAAGGAATGAAA
GGGATGGACAATAATGCACCACACGGTTTTTTCGGAGGTGGATCGGATAGTTAT
GAACAAATGATGTTAATGGGTGGAGCTGGTCAACAAGGATATGGCGGAATTCC
GAGCTGGATGGGTGGATCACCTGGAGGGTTTTCAACATCAGTAGCTGGCGTACA
AACAGGAATGCCTACATCAATGGGGGGCTTTTCGACATCAGGGGCTGGAATGCC
TGCAGTAACAGGAGGCTTTCCAACGTCAGTAGCTGGCGTACAGACAGGATTTCC
AGTACCAGGAGTAGGAGTTGTAGCTGGAGGA|\overline{ATAGGTGGTTTTCCGCAAGTTAT}|
|\overline{TC}|ATGGTCATCATGGACATCACCACCATCAAGGTCACCACGGTCATCATGGACA
TCACCACCATCAAGGTCACCACGGTCATCATGGACATCACCACCATCAAGGTCA
CCACGGTCATCAAGGCCACCATCATCACCAAGGTCACCACGGTCATCATGGGCA
TCATCAACAACAAGTACATCACCACGGCCATCATCATATACATC|\overline{CACAGGCTAT}|
|\overline{TCTTTATCAAACTCATC}|AAGGTCACCAAGGCCACCATGATCATCACGGTCACCA
CGGTCATCAAGGCCACCACGGTCAACACAGTCAACAGCACCAACAGCACCAAC
AATATCAACAATATCAACAATATCAACAATATCAA|\overline{CAACAATCTTCACCTTGGG}|
|\overline{CAGG}|GGAATTGGAGCAGGGGCAGCGGGGGCAGCGGCAGGAGCGGCTAGTATTA
AAGACATAATAAAAAACTGCTAGGAGTTCCTAGCAGTTTTTTTCAGCTTCTTGGC
GTTGACGTTCGATTTCAGCACGAAGTTGTGGTTCTGGTGCTTGCCAGCCTTCAGG
TTTTAGAATTTTCCCGTCACCTTCGCGGAATCGAGGTTTCCCGTCTGGGAATAGT
TTTGCCATGTTTGCATTATTTACAATTTCGAATCCTTTATCAGGACGTACGCCCA
TTTCGGCAAACGTGCCGAATGCAAAGTAAATAAGATCAATTAGTGCATCGTATT
GATCTTCAACAGTCGTTGCTTCTAGAATTCTTCTAGCTCTTCTTGCATAAAACT
CGCACGGATTTTTGCACGTTCTTCTGTTAATTTTGTTGGAGTATTTGTCACAGGA
TGTCCGAATACTTCATGCATTTTTGCACAAGTTCGTACCCTTTATCTAAACCTTT
TTCGTTTGTCATGTTGATTCATCCTTTCTCGCTTTGTGCCGAATCTCATTGTAACA
TAAGCTGATTATGAAATGAAAAGGGACTGTTTAAAAGTTTCATCTTTTGGTTAAG
GTAGGGAAATATTAT
        b) CG3 variable sequence (SEQ ID NO:18):
        ..GAATTCCGTAATCCGATACTAAATATAAAAGTTCTTCTTTCTTTATTTCA
ATTACTTTTTCCAATTTCACTAATTCCA|\overline{TGTCGTTTTACTTCTCTCTCCAATAC}|TA
AAATGTATGTATTTTAGTATAGCCTATTTACATAATTAGTAAAAGTAAAATATGC
ATGTAAAATCTTACAAATATGAAAATAATATAATATCACCAAATAAATTT
|\overline{ATGCCCTTTATACAGAACAATGACT}|CATCTCATATTCATAACAAATCGGAATTTC
TCTAAAGTTATATCTTTATATTTTGGAGCCCTCTTACTTAA
        c) GG2 variable sequence (SEQ ID NO:19):
        |\overline{GAATTCGGCGTAACTGC}|TGCTCCTGTAGCTGTTGCTGGTGGCGCTGGAGA
AGCTGCTGCTGAGAAAACTGAATTTGATGTGGAACTAACTAGCGCTGGTGCACA
AAAAATCAAAGTTATCAAAGTTGTTC|\overline{GTGAAATCACTGGTCTTGGCTT}|AA
        d) AAT repeat from pXO1 (SEQ ID NO:20):
        TAAAAAATTCATTGATATTTTTAAGGAAGAAGGGAGTAATTTAACTTCGT
ATGGGAGAACAAATGAAGCGGAATTTTTTGCAGAAGCCTTTAGGTTAATGCATT
CTACGGACCATGCTGAACGTTTAAAAGTTCAAAAAAATGCTCCGAAAACTTTCC
|\overline{AATTTATTAACGATCAGATTAAGTTCA}|TTATTAACTCATAAGTAATGTATTAAAA
ATTTTCAAATGGATTTAATAATAATAATAATAATAATAATAACGGGACCA|\overline{GCCA}|
|\overline{TTATGAAGCAACTAATTCTAGA}|CTTGATAGTAATTCTTGGGAAGCACCAGATAG
TGTAAAAGGTGGCATTGCCAGAATGATATTTTATGTGTTCGTTAGATATGAAGG
CAAAAACAATGATCCTGACCTAGAACTTAATGAT
        e) AT repeat from pXO2 (SEQ ID NO:21):
        CCTAAGTAATAAACCTCATAAATCTGTCGCCAGTTACAACGTAGCACCCA
AAGATAAGGCATGTCAATTGTACAAATTATAAATTTTAATATATGAATCACAAAT
TATAGGATATTTACTATACATTCTTTTGTATTGATATTTTTAAGCCCTCTTCTAA
AAAACGGCTCTTTATTTTCGTTTTTAACGCTTTTTACAACTACCATTAATACATTT
GCACCAAAAAGAGGAAAAATCATAAAAACGAATTAATAGCCCCCTTAAATGGA
ATTCCTATAAAATCATTTCAAATTATTGTTT|\overline{TCATCCTCTTTTAAGTCTTGGGT}|TA
TATTTATCTACCTTTGCGGTATGTGAAATTATATATATATATATCAAAAGAAAAAA
AGGAAGGGGAATACCCCCCTCCTTTACTTGTACTTT|\overline{TGTCGTCGGAGTTCATCAC}|
|\overline{AC}|TTGGACAATATACAAAGACCCATTCCATTGTAATTGTACGTCGCCCTGTTGA
AAGCCAGACAACACGCCATGCAGCGCCCCCACATTTATTGCAGCGGTACACCTC
ATTGTGATTTACCTCAAAACCAATTTGTTTCTGTTTCATTTAAAATCTCTCCTTTGC
        f) vrrC variable sequence (SEQ ID NO:22):
        GAGCGACAATATGAAGAGAGTAAAAAGGAAGAAAAGTCAGTTGTCGATC
AATGGCTTGAGAAAAACGGTTATGAAATCGAACGCCAAGAGCCTATAGTAGAA
GAAAAAGAAGTGGTTCAAGAAATGAGTGCACCGCAGGAAGTTCCAGCGGcTGA
ATTACTTCATGAAACAATTGCTGAGCGCATGGAAGGTGC|\overline{GAAGCAAGAAAGTG}|
|\overline{ATGTAGTGGAC}|AAAAACATTCTACAAGAGGAATTAGTAGATTCTAAAGTAGAG
CACGAGGATACAATATTATCAGAAGAAATTAAACGTAGTACAGAAATAGAACA
ACCTACTATTGAAGTAGAAAAACAAGCACCAGAAGAATCAGTGATTGTCAAAG
CAGAAGAAAAACTTGAAGAAACAATTGTTGTAGAAATACCAGAAGAAGTGGAA
GTAATTGCAGAAACAGAAGAGTCAGAAGAAGTAGAAGTAATTGCAGAAACAGA
AGAGTCAGAAGAAGTAGAAGTAATTGCAGAAACAGAAGAGTCAGAAGAAGTAG
AAGTAATTGCAGAAACAGAAGAGTCAGAAGAAGTAGAAGTAATTGCGGAAACA
GAAGAGTCAGAAGAAGTAGAAGTAATTGCAGAAACAGAAGAGTCAGAAGAAGT
AGAAGTAATTGCAGAAACAGAAGAGTTAGAAGAAGTGGAAGTAACTGCAGAAA
CAGAAGAATTAGAAGAAGTGGAAGTAGTTGCAGAAACAGAAGAGTTAGAAGAA
GTGGAAGTAATTGCAGAAACAGAAAAATTAGAAGAATTAGAAGAAGTGGAAGT
AATTGCAGAAACAGAAGAGTCAGAAGAAGTAGAAGTAATTGCAGAAACAGAAG
CA|\overline{CCAGAAGAAGTGGAACCTGTAGCACTTGAGGAAATG}|ACAAGAAATGGTGTT
AAATGAAGCAATTGAACAAAAGAATGAATTCATACATGTTGCTGTGGCTGATGA
ACAAACGAAAAAAGATGTTCAAAGCTTTGCGGATGTTTTAATTGCGGAGGAACA
ATCAGTTGTAGAAGAAACACCGATTGTAGAAGAACAACCAGTTGCAGAAGAAG
CACCGGTCGTAGAAGAACAATCAGTTGTAGAAGAAACACCGATTGTAGAAGAA
CAACCAGCTGCAGAAGAAGCACCGGTCGTAGAAGAACAATCAGTTGTAGAAGA
AACACCGATTGTAGAAGAAGCACCGGTCGTAGAAGGACAATCAGTTGTAGAAG
AAGCACCGATTGCAGAAGAACAACCAGTTGCAGAGGAAACATCGGTCGTAGAA
GAACAACCAGTTGCAGAGGAAACATCGATCGTAGAAGAACAACCAGTTGCAGA
GGAAGCACCAGTCGTAGAAGAACAACCAGTTGTACAAAAAGAAGAACCAAAAC
GTGAGAAAAAGCGTCACGTACCATTTAATGTTGTTATGTTGAAACAAGATAGAG
|\overline{CGCGATTAATGGAAAGAC}|ATGCGTCTAGAACGAATGGAATGCAATCTTCTATGA
GTGAAAGAGTAGAGAATAAGCCTGTACATCAAGTAGAAGAACAACCACAAGTG
GAAGAAAAGCCAATGCAACAAGTAGTAGTGGAGCCACAAGTGGAAGAGAAAC
AAATGCAGCAAGTAGTGGAGCCACAAGTGGAAGAAAAACCAATGCAACAAGTA
GTAGTGGAACCACAAGTGGAAGAAAAACCAATGCAACAAGTAGTAGTGGAGCC
ACAAGTGGAAGAAAAACCAATGCAGCAAGTAGTGGTAGAACCGCAAGTGGAAG
AAAAACCAATGCAACAAGTAGTAGTGGAGCCACAAGTGGAAGAAAAACCAATG
CAGCAAGTAGTGGTAGAACCGCAAGTGGAAGAAAAACCAATGCAGCAAGTAGT
GGTAGAACCGCAAGTGGAAGAAAAACCAGTGCAGCAAGTAGTAGAGCCACAAG
TGGAAGAAGTGCAGCCAGTACAACAAGTAGTGGCAGAGCAAGTACAAAAGCCA
ATTTCAAGTACGGAAGTAGAAGAGAAAGCATATGTTGTAAATCAAAGAGAGAA
T
```

*Note
that the vrrB₁-r1 and the vrrB₂-f1 primer sequences overlap.
*Note
that the vrrC₁-r1 and the vrrC₂-f1 primer sequences overlap.

PCR amplification products from the eight VNTR regions were detected and sized using fluorescently labeled primers on an automated DNA sequencer. A collection of 425 isolates were MLVA characterized to identify 89 distinct genotypes. VNTR markers frequently had multiple alleles (from 2 to 8) and diversity (D) values between 0.3 and 0.8. UPGMA cluster analysis identified six genetically distinct groups that appear to represent genetic clones. Some of these clones show worldwide distribution, while others are restricted to particular geographic regions. Human commerce has been involved in the dispersal of particular clones in ancient and modern times. Because of the nearly monomorphic molecular nature of B. anthracis, MLVA may be the only reasonable method for understanding the diversity, evolution and molecular epidemiology of this pathogen.

Although the function of these genetic sequences is unknown, the hypermutability of these genetic elements could have great biological importance to these bacteria. Thus, it is likely that gene products from these DNA sequences may be useful for therapeutic, diagnostic or industrial applications.

Reference will now be made in detail to the preferred embodiments of the present invention, examples of which are illustrated in the accompanying Figures.

A. DNA Preparation: 426 B. anthracis isolates from around the world (Table 3) were analyzed.

TABLE 3

| | | No. | No. |
|---|---|---|---|
| Continent | Country | Isolates | Genotypes |
| Africa | Mozambique | 5 | 4 |
| | (MOZ) | | |
| | Namibia (NAM) | 23 | 7 |
| | South Africa | 127 | 9 |
| | (SAF) | | |
| | Tanzania (TANZ) | 5 | 1 |
| | Zambia (ZAM) | 17 | 2 |
| | Zimbabwe (ZIM) | 4 | 2 |
| | Totals = | 181 | 18 |
| Asia | China | 7 | 5 |
| | India | 3 | 2 |
| | Indonesia (INDO) | 5 | 4 |
| | Pakistan (PAK) | 4 | 4 |
| | S. Korea (KOR) | 4 | 2 |
| | Turkey (TURK) | 41 | 12 |
| | Totals = | 64 | 31 |
| Australia (AUS) | Totals = | 30 | 3 |
| Europe (EUR) | Croatia (CRO) | 1 | 1 |
| | France (FRA) | 8 | 3 |
| | Germany (GER) | 9 | 5 |
| | Hungary (HUN) | 3 | 2 |
| | Ireland (IRE) | 1 | 1 |
| | Italy | 3 | 2 |
| | Norway (NOR) | 5 | 5 |
| | Poland (POL) | 1 | 1 |
| | Slovakia (SLO) | 3 | 2 |
| | Spain (SPA) | 2 | 1 |
| | Switzerland (SWI) | 2 | 2 |
| | United Kingdom | 19 | 10 |
| | (UK) | | |
| | Totals = | 57 | 32 |
| North America | Canada (CAN) | 51 | 7 |
| (NoA) | | | |
| | Haiti | 1 | 1 |
| | United States | 32 | 16 |
| | (USA) | | |
| | Totals = | 84 | 22 |
| South America | Argentina (ARG) | 2 | 2 |
| (SoA) | | | |
| | Brazil (BRA) | 1 | 1 |
| | Totals = | 3 | 3 |
| | Overall Total = | 419 | 89 |

These samples include previously described samples (See, “Characterization Of The Variable-Number Tandem Repeats In vrrA From Different Bacillus anthracis Isolates” by Paul J. Jackson et al., Appl. Environ. Microbiol. 63, 1400 (1997), “Molecular Evolution And Diversity In Bacillus anthracis As Detected By Amplified Fragment Length Polymorphism Markers” by Paul S. Keim, J. Bacteriol. 179, 818 (1997)), plus more than 300 additional clinical or environmental isolates (Table 1). DNA from each isolate was obtained by either large batch procedures (See, Jackson et al. 1997, supra, and Keim et al. 1997, supra.) or by a greatly simplified approach requiring only heat lysis of a single colony. In this abbreviated protocol, B. anthracis cells were streaked onto blood agar plates, and then incubated at 37° C. overnight. A single colony from each plate was transferred into a microfuge tube containing 200 μl of TE (Tris HCl, pH=8.0, 1.0 mM EDTA). The colony was resuspended by vortexing or repetitive pipetting. The cellular suspension was heated to 95° C. for 20 minutes, and then cooled to room temperature. Cellular debris was removed by centrifugation at 15,000×g for 1 minute. The supernatant was then transferred to a new tube for storage. 1 μl of the lysate contained sufficient template to support a single PCR reaction, which means that this procedure can supply template for 200 reactions. Reactions were periodically optimized by titrating the heat lysate template concentrations using serial dilutions. Both DNA preparation protocols gave the same MLVA results; however, the heat lysis procedure is much more rapid and easily adapted to large-scale processing of samples.

B. MLVA PCR: MLVA reaction primers (Table 1) were designed to provide uniquely labeled or sized amplicons for every allele at the eight VNTR loci. PCR amplification of all eight VNTR loci was routinely accomplished using four reactions. Two of the amplicons (vrrC₁ and vrrC₂) are significantly larger than the others and, in addition, were amplified using partially complementary primers. Likewise, vrrB₁ and vrrB₂ were amplified using complementary primers. Limited unique sequences in these repeated regions necessitated the overlap of these primers and, thus, required these amplicons to be divided into separate PCR reactions. Large amplicons tend to be outcompeted by small amplicons and, thus, require separate PCR reactions. These restraints led to a four reaction design where: (1) vrrB₁ was grouped with CG3 and vrrA; (2) vrrB₂ was grouped with pXO1-aat and pXO2-at; (3) vrrC₁ was amplified alone; and (4) vrrC₂ was amplified alone.

Reaction 1 contained: 1×PCR buffer (20 mM Tris-HCl pH=8.4, 50 mM KCl); 2 mM MgCl₂; 0.2 mM dNTPs; 0.04 U/μl Platinum™ Taq DNA polymerase (Gibco-Life Technologies); 0.1 μM CG3-F1 and CG3-R1; 0.2 μM each of vrrA-F1; vrrA-R1, vrrB₁-F1 and vrrB₁-R1; 0.04-0.2 ng/μl of template DNA or simply 1 μl of the single colony lysate.

Reaction 2 contained: 1×PCR buffer; 4 mM MgCl₂; 0.2 mM dNTPs; 0.04 U/μl Platinum Taq DNA polymerase; 0.4 μM each of vrrB₂-F1, vrrB₂-R1, pXO1-aat-F1, pXO1-aat-R1, pXO2-at-F1 and pXO2-at-R1; 0.04-0.2 ng/μl of template DNA or simply 1 μl of the single colony lysate.

Reaction 3 contained: 1×PCR buffer; 2 mM MgCl₂; 0.2 mM dNTPs; 0.04 U/μl Platinum Taq DNA polymerase; 0.2 μM each of vrrC₁-F1 and vrrC₁-R1; 0.04-0.2 ng/μl of template DNA or simply 1 μl of the single colony lysate.

Reaction 4 contained: 1×PCR buffer; 2 mM MgCl₂; 0.2 mM dNTPs; 0.04 U/μl Platinum Taq DNA polymerase; 0.2 μM each of vrrC₂-F1 and vrrC₂-R1; 0.04-0.2 ng/μl of template DNA or simply 1 μl of the single colony lysate.

The PCR thermocycling program for all four reactions was identical. Once the reactions were assembled, they were heated at 94° C. for 5 min. to activate the DNA polymerase. Thereafter, each temperature cycle was 94° C. for 20 s, 60° C. for 20 s, and 65° C. for 20 s. These three steps were repeated 34 times. The final step was at 65° C. for 5 minutes.

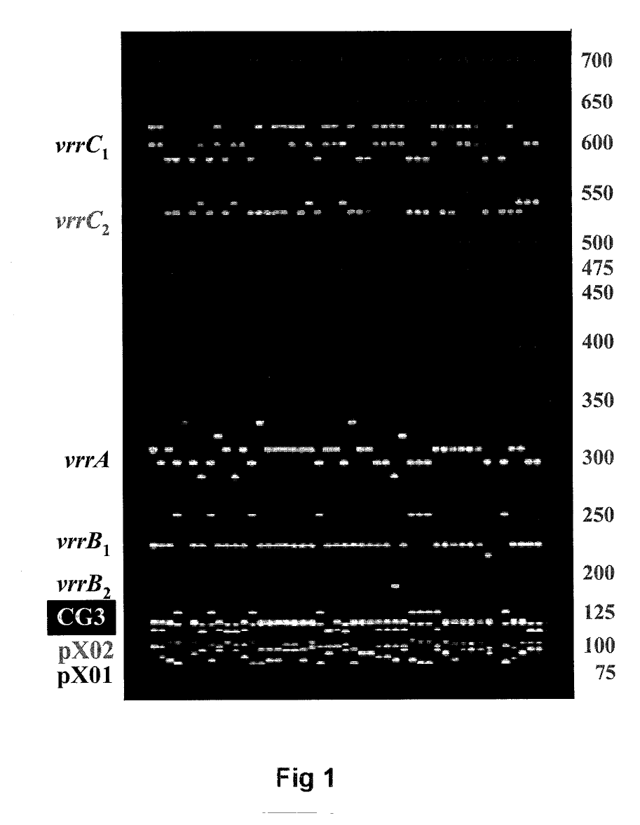

C. Automated Genotype Analysis: The MLVA PCR reactions products were electrophoretically analyzed using a Perkin-Elmer Applied Biosystems 377 automated fluorescent DNA sequencer. The results are shown in FIG. 1. Each lane contains a random strain chosen from the worldwide diversity set shown in Table 3. The four reactions were mixed in equal amounts prior to electrophoretic analysis, which provides relatively equal fluorescent signal from each amplicon. Genescan™ and Genotyper™ software packages (Perkin Elmer, ABI) were used to analyze the gel images. Custom macro programs (available upon request) associated with Genotyper™ allow the automated scoring of alleles.

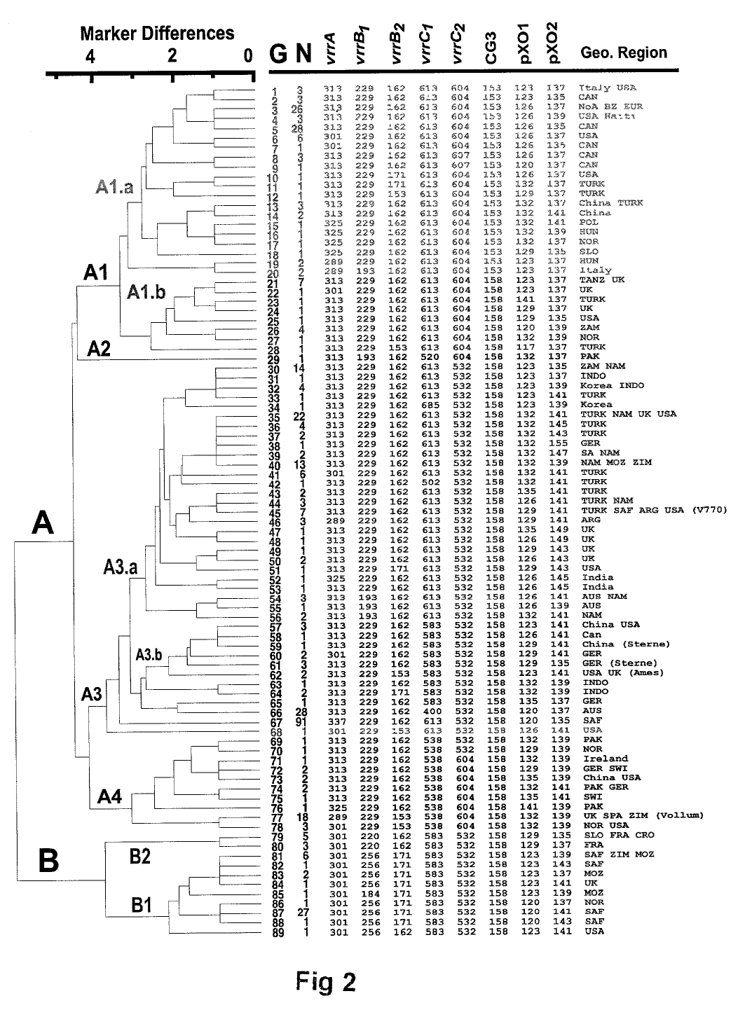

The apparent electrophoretic size of DNA fragments was not always exactly the same as the size determined by DNA sequencing. This could be due to DNA conformational differences, 3′ adenine addition by the polymerase, migrational deviations of the size standard, or mass asymmetry between the amplicon strands that affect the comparison with the single stranded standards. The actual nucleotide sequence of most marker alleles was determined by DNA sequencing and these values are reported in all cases. These differences are usually only one or two nucleotides, but the use of standard genotypes selected from FIG. 2 are recommended as references.

D. Data Analysis: Only genotypes generating data from all eight markers were included in these analyses. About 5% of the samples examined were missing one or both virulence plasmids, which precludes complete genotyping using the MLVA method of the present invention. This includes the commonly used vaccine strains that lack the pXO2 plasmid. These strains are annotated on FIG. 2 next to their seven-marker genotypic matches. The eight VNTR marker loci were used to calculate a matching coefficient among all 89 unique MLVA genotypes, and UPGMA cluster analysis was performed to identify groups of similar genotypes from the worldwide collection. The genetic distance is presented as the absolute number differences in marker alleles between genotypes. The amplicon sizes presented are based upon nucleotide sequence determinations using the primers from TABLE 1. Country abbreviations are defined in Table 1. The vaccine strains, Sterne, STI-1 and V770-NP1 are lacking the pXO2 plasmid marker and were not included in the cluster analysis; however, the data set was annotated (see Geo. Region column) to indicate where these strains match other genotypes based upon seven marker loci. STI-1 did not match any of the genotypes. Genotypes of the well-known strains, Ames and Vollum are labeled. Marker alleles are presented as their sizes in nucleotides. VrrA allele 313 corresponds to VNTR₄ described previously (See, “Characterization Of The Variable-Number Tandem Repeats In vrrA from different Bacillus anthracis Isolates”, by Paul J. Jackson et al., Appl. Environ. Microbiol. 63, 1400 (1997)).

Analysis of the raw genotype scores was accomplished by using a phenetic approach: Unweighted Pair Group Method Arithmetic average (UPGMA) cluster analysis (See, “Genetic Data Analysis” by B. S. Weir Sinauer Associates, Inc. Sunderland Mass. (1990)). UPGMA cluster analysis was performed using PAUP 4.0 (See, “PAUP-Phylogenetic Analysis Using Parsimony And Other Methods, 4.0 Beta Version” by D. Swofford, Sinauer Associates, Inc. Sunderland Mass. (1999)) with a simple matching coefficient to estimate genetic differences. Individual marker diversity (D) was calculated as equal to 1−Σ(allele frequency)² (B. S. Weir, supra) and based upon allele frequencies in the 89 distinct B. anthracis genotypes, not the complete 426 isolate collection.

E. Multiple-Locus VNTR Analysis (MLVA): A multi-locus VNTR analysis (MLVA) approach for molecular typing of B. anthracis strains has been developed. The present invention utilizes eight genetic loci that provide high levels of discrimination among different isolates. These marker loci were identified by DNA sequencing of variable AFLP marker fragments (CG3, vrrB₁, vrrB₂, vrrC₁ and vrrC₂), examination of virulence plasmid sequence (pXO2-at and pXO1-aat) and from the previously described vrrA VNTR locus (See, “Identification Of A Region Of Genetic Variability Among Bacillus anthracis Strains And Related Species” by G. L. Andersen, J. Bacteriol. 178, 377 (1996)). Five of the eight MLVA markers (vrrA, vrrB₁, vrrB₂, vrrC₁ and vrrC₂) are found in ORFs and variation in repeat number does not affect the translational reading frame (data not shown). The pXO1 and pXO2 VNTR markers allow monitoring for the presence or absence of the plasmids, as well as for plasmid-based variation. This plus-minus assay provides important information about virulence because the lack of either plasmid attenuates a B. anthracis strain (See, “Bacillus anthracis by C. B. Thorne, p. 113-132, In A. L. Sonenshein et al. (eds.), Bacillus subtilis And Other Gram-Positive Bacteria: Biochemistry, Physiology, And Molecular Genetics, American Society for Microbiology, Washington, D.C. (1993)). Phylogenetic comparison of nucleotide variation did not detect natural horizontal transfer among strains (See, “Natural Genetic Diversity In The Protective Antigen Gene Of Bacillus anthracis” by L. B. Price, J. Bacteriol. 181:2358 (1999)), suggesting that plasmid and chromosomal evolution in B. anthracis has been generally congruent.

While no effort was made to make the MLVA primers specific to B. anthracis templates, most sets will not support amplification from other bacterial species. A limited number of B. cereus and B. thuringiensis strains have been examined using the standard reaction conditions; at most couple, and frequently none, of the markers amplified in reactions containing these templates (data not shown). The vrrA locus amplified most often in other species, but the resulting allele sizes did not correspond to any of the five alleles observed in B. anthracis isolates. These bacterial taxa are the most closely related bacilli to B. anthracis. Therefore, this MLVA system represents a credible method of identifying B. anthracis, as well as, determining what strain type is present.

F. B. anthracis Genotypes: MLVA was used to characterize 426 B. anthracis isolates from diverse geographic locations. This analysis divided them into 89 MLVA genotypes (FIG. 2). It is clear that multiple isolates from the same anthrax outbreak frequently have identical genotypes. This reduces the number of distinguishable isolates relative to the total number of samples. In addition, many genotypes are found at multiple locations especially within a restricted geographical region. The number of distinct genotypes collected from particular countries is reported in Table 3. The distribution may be more a function of isolate availability for this study than intrinsic diversity within a limited geopgraphic area, so it is difficult to draw conclusions from these numbers. However, multiple genotypes are observed from all regions where a large collection of samples are available. The Australian collection is heavily biased by 28 samples from the short 1997 Victoria outbreak. All of these are one genotype. The restricted nature of the collection may, therefore, explain the lack of multiple genotypes discovered to date in Australia.

G. VNTR marker diversity: The discriminatory power of each MLVA marker can be estimated by the number of alleles it detects and by its diversity. These two simple descriptive statistics were determined using only the 89 B. anthracis genotypes to minimize the affect of sampling on allele frequency (Table 4). The isolate collection is biased towards numerous samples from easily accessed B. anthracis collections. This could unduly influence allele frequencies. MLVA markers average over five alleles per locus with a range from two to nine alleles. TABLE 4 shows VNTR marker loci attributes.

TABLE 4

| Marker | Repeat size^{§} | Array Size (repeats) | | Number of | D^{‡} |
|---|---|---|---|---|---|
| Name* | (nucleotides) | Smallest | Largest | Alleles | (Diversity) |
| VrrA | 12 | 2 | 6 | 5 | 0.50 |
| VrrB₁ | 9 | 15 | 23 | 5 | 0.32 |
| VrrB₂ | 9 | 11 | 15 | 3 | 0.34 |
| VrrC₁ | 36 | 4 | 12 | 6 | 0.55 |
| VrrC₂ | 18 | 17 | 19 | 3 | 0.50 |
| CG3 | 5 | 1 | 2 | 2 | 0.35 |
| pX01-aat | 3 | 4 | 11 | 8 | 0.81 |
| pX02-at | 2 | 6 | 15 | 9 | 0.79 |
| Average | — | — | — | 5.1 | 0.52 |

*VNTR markers found in ORFs are shown in italics.
^{§}VrrB repeats are not all identical as some contain single nucleotide differences.
^{§}VrrC markers contain a degenerate 9-nucleotide sub-repeat structure that results in some alleles of fractional repeat size.
^{‡}D is marker diversity. calculated as 1-Σ(allele frequency)² and based upon the 89 unique B. anthracis genotypes.

The diversity index (D) is based on the number of alleles and the allele frequency. This provides a better measure of discriminatory power than allele number (See B. S. Weir, 1990, supra.). MLVA markers have an average diversity of 0.54 with a range of 0.30 to 0.80. Note that vrrB₁ has the lowest diversity (0.30) in spite of having five alleles; whereas, CG3 detects only two alleles but has a diversity index of 0.38. The two plasmid-based markers have the highest diversity and greatest number of alleles, perhaps due the simple sequence nature of their repeats (Table 4).

While most of the observed B. anthracis allelic variation is consistent with the repeat unit size, some alleles contain fractions of a repeat. The nucleotide structures found in the vrrA, vrrB and vrrC have evolved from simpler trinucleotide repeats and remnants of these structures still exist within each repeat (See, G. L. Andersen et al., 1996, supra, and unpublished data). No fractional-size alleles were observed for vrrA or vrrB among the different B. anthracis strains, but several were observed for the vrrC markers. Nucleotide sequencing determined that these were due to insertion/deletion events within the subrepeats (See the vrrC₂ alleles in genotypes 8 and 9 of FIG. 2.).

H. B. anthracis Genetic Relationships: UPGMA cluster analysis reveals major genetic affiliations among the MLVA genotypes (FIG. 2). Six major clusters are apparent that may represent older clonal separations in the evolutionary history of this species. Similar major groups were identified using AFLP markers analysis (See, Paul S. Keim et al., 1997, supra.), most of which were independent of the MLVA markers.

The most obvious separation in the dendrogram is the split between the A and B genotypes (FIG. 2). The B cluster contains approximately 12% of the isolates and genotypes in this study. Cluster B is further subdivided into two groups, B1 and B2. Southern African isolates dominate (93%) group B1 and far outnumber samples found in group B2. Only two genotypes are present in the B2 group. These are rare and collected exclusively in Europe. The B2 group is only tentatively associated with the B1 subgroup as other analytical approaches (e.g. maximum parsimony) place B2 loosely with the A cluster (data not presented). All B genotypes are uncommon in much of the world, yet genotype 87 (FIG. 2) is an important contributor to the ongoing anthrax outbreak in Kruger National Park (See, “Bacillus anthracis Diversity In The Kruger National Park” by K. L. Smith et al., J. Clinical Microbiol. 38, 3780 (2000)).

Members of the A cluster are found worldwide and can be subdivided into at least four groups (FIG. 2). Isolates in the A1 cluster are found throughout the world, but they dominate the western North America collection. The most common A1 genotypes are geographically distributed from the Canadian Wood Bison National Park (genotypes 3 and 5) to southern Texas in the United States (genotype 8). The CG3 marker locus represents a defining diagnostic marker for the A1.a group as the 153 bp allele is only found in this group. This marker locus consists of a five-nucleotide sequence present in two copies in most strains, but only once in isolates found in cluster A1.a. This difference may not be readily reversible and all allelic contrasts due to a single evolutionary event. While STI-1 was not included in the UPGMA analysis due to its lack of the pXO1 plasmid, it most closely resembles members of the A1.a group. As the sole representative from Russia in this study, it did not exactly match any of the 89 genotypes with its seven markers. However, it is clearly related to isolates from the A1.a cluster. STI-1 marker alleles (FIG. 2: 313, 229, 162, 13, 604, 153, 129,- - -) matched six of seven markers for 11 different genotypes in A1.a. In addition, STI-1 contains the CG3 153 allele that is only present in A1.a isolates. The close genetic relationship between the western North American isolates and this single Russian representative needs further research and would benefit significantly from examination of additional Russian isolates. The A1.b cluster isolates occur most commonly in Africa and only rarely in other parts of the world.

The A2 branch is represented by a single isolate from Pakistan. It is distinct from other genotypes and may represent a B. anthracis that is common in this under-sampled region.

The A3 cluster is perhaps the single most important B. anthracis group due to its wide distribution and prevalence. This highly diverse cluster contains 44% of the genotypes (39 of 89) and 58% of the isolates (260 of 419) examined in this study (FIG. 2). Genotypes in this group are involved in some of the largest outbreaks that we have examined: Kruger National Park (genotype 67), Victoria Australia (genotype 66), Turkey (genotype 35) and southern Africa (genotypes 30 and 40). Genotypes matching the well-known vaccine strains V770-NPR (genotypes 45, 46 and 49) and Sterne (genotypes 59 and 61) are also found in this cluster. The well-known and highly virulent strain, Ames (genotype 62), is found in A2 and is similar to Sterne at most marker alleles. The Ames strain played a central role in the United States biological warfare program, before it was dismantled.

The A4 cluster is distinct and, yet, underrepresented in our current collection (FIG. 2). It is notable primarily for the well-known strain, Vollum (genotype 77), which was used in the British biological warfare program (pers. com. Peter Turnbull). Vollum has been studied in many laboratories and most of the fifteen isolates identical to genotype 77 are from laboratory archival collections. One sample of the Vollum 1 B strain differed by at the vrrA markers by one repeat from other Vollum samples. This seemingly represents an “in laboratory” mutational event. A natural isolate matching the Vollum genotype was collected in Spain. Other closely related isolates have been found in the USA, Norway, Europe and Asia, but not in Africa.

The MLVA typing method of the present invention represents a robust and easily transferable approach to characterizing B. anthracis isolates. The protocols presented are rapid and require only crudely isolated DNA to provide high-resolution molecular typing analysis. The individual marker alleles are uniquely identified by a combination of size and fluorescent color. Therefore, automated gel analysis is routine, and instrumentation for performing MLVA is widely available. Standardized data have been presented in order to provide a uniform reference to all anthrax research teams (FIG. 2). Future analyses by any laboratory in the world can be easily compared to these standardized data and particular strains (Table 4).

Molecular typing in many pathogenic bacterial species is accomplished without focusing on hypervariable VNTR loci. In B. anthracis, however, this has proven extremely difficult due to the homogeneous nature of all available strains (See, Paul S. Keim et al., 1997, supra.). In this pathogen, only the most rapidly evolving genomic regions are useful for strain discrimination. VNTR loci fall into this category and have successfully been used in this study to separate B. anthracis isolates into 89 distinct genotypes. As a first approximation, one can assume that the diversity of a particular VNTR is correlated with its evolutionary rate and, in the absence of selective constraints, this would be the mutation rate. The simple di- and tri-nucleotide tandem repeats (pXO1-aat and pXO2-at) are found to be the most diverse, while complex longer repeats have been found to have lower diversity (Table 4). Slip strand repair mutations by DNA polymerase are thought to occur more frequently on short repeats (See, “Short-Sequence DNA Repeats In Prokaryotic Genomes” by A. van Belkum et al., Microbiol. Mol. Biol. Rev. 62, 275 (1998).) and the present data are consistent with this model. Markers of higher diversity obviously provide great discriminatory power among strains. However, it is less obvious that highly diverse markers have less power for defining older evolutionarily relationships. The present MLVA markers have different diversity levels and contribute in different ways to the analysis of B. anthracis.

VNTR mutation rates are apparently fast on an evolutionary scale, but slow enough that mutations are very difficult to observe in the laboratory. A plasmid-cured strain of Ames was passaged for more than 100,000 generations and only one VNTR mutation (313 to 301) in vrrA was observed (G. Zinser and P. Keim, unpublished observations). At least in the six chromosomal loci, marker alleles appear stable to routine and even long-term handling in the laboratory. As stated above, variation in different Vollum strain (Genotype 77) accessions illustrate the stability of these markers. There are 15 different Vollum examples in this study from different sources in the USA and the UK. One of these was passaged three times through rats and three times through rabbit hosts, without MLVA changes. The only difference was observed in the vrrA marker (301 instead of 289) for the substrain Vollum 1B. These anecdotal and preliminary results need additional confirmation but suggest that VNTR mutation rates are slower than 10⁻⁵ and that mutational changes occur in single repeat steps.

The existence of a limited number of B. anthracis clones can be hypothesized from the genetic similarities observed within each of the six major clusters in FIG. 2. The number and distribution of these hypothesized clones has doubtlessly been influenced by human activity. This may have started with domestication of animals, but continues through modern day international commerce. Note that not all of the putative clones are equally widespread. For example, the A3 cluster is very common and distributed across many continents, while the B1 cluster is restricted mostly to southern Africa. The A1 cluster splits into two distinctive groups, with one branch primarily North American (A1.a) and one mostly African (A1.b). These differences in cluster prevalence and distribution may be influenced by inherent biological properties or just due to stochastic historical events.

While the biological significance of B. anthracis VNTR variation is unknown, some VNTR variation examples have pronounced effects upon pathogen biology (See, A. van Belkum et al., 1998, supra.). Five of the eight MLVA loci in the MLVA system are found in ORFs (data not presented). Therefore, VNTR variation could easily affect the bacterial phenotype via altered translational products. Moreover, it has also been shown that extra-genic VNTRs can influence adjacent gene expression (See, A. van Belkum et al., 1998, supra.). This provides a possible genetic role for even intergenic VNTRs. Given the extreme homogeneity of B. anthracis, VNTRs represent the only significant source for molecular variation among strains known at this time.

Tables 5 and 6 show additional loci having variable numbers of repeat units which were discovered by examining the B. anthracis DNA sequence. Alleles are detected by directly incorporating the dyes into the PCR amplicon rather than having dye labels on the associated primers. Although these loci have not yet been employed for strain identification, it is expected that they will be useful therefor.

Table 5 shows the additional primers expected to be useful for DNA fingerprinting of B. anthracis.

TABLE 5

| | | | | | | |
|---|---|---|---|---|---|---|
| | | | | Amplicon | | |
| | Primer Sequences | Repeat | Array Size | Size | | |
| Locus | (Forward primer on top) | Size (bp) | Range | Range | Alleles | D |

| | | | | | | |
|---|---|---|---|---|---|---|
| vrrC3 | hex-gacatgcgtctagaacgaatggaatgcaatcttc | * | | | | |
| | (SEQ ID NO:23) | COMPLEX | ** | 320-528 | 3 | 0.15 |
| | cttccgtacttgaaattggcttttgtacttgctct | | | | | |
| | (SEQ ID NO:24) | | | | | |
| Bavntr- | agtatagcaaaggcaataatgaat | COMPLEX | ** | 609-854 | 4 | 0.28 |
| 11 | (SEQ ID NO:25) | | | | | |
| | cgcaaggaaatatgaaatcag | | | | | |
| | (SEQ ID NO:26) | | | | | |
| Bavntr- | gaaaaagtacggagtggattttgatacgctgaaac | COMPLEX | ** | 515-540 | 3 | 0.07 |
| 22 | (SEQ ID NO:27) | | | | | |
| | ggtggctgtggtaacacatttaccgaaaac | | | | | |
| | (SEQ ID NO:28) | | | | | |
| Bavntr- | gaaatcaatgacgaaaattttcattttagcatcaactc | COMPLEX | ** | 553-821 | 8 | 0.47 |
| 31 | (SEQ ID NO:29) | | | | | |
| | gccttgttttttcgcattatcttttacattaccacc | | | | | |
| | (SEQ ID NO:30) | | | | | |
| Bavntr- | gagccagcataacgctagctctgttttg | 2 | 5 to 7 | 246-250 | 3 | 0.11 |
| 14 | (SEQ ID NO:31) | | | | | |
| | gatatgacgggtggtatgaattggaaaggtac | | | | | |
| | (SEQ ID NO:32) | | | | | |
| Bavntr- | gagttaaaccataaggaatacacc | COMPLEX | ** | 501-584 | 6 | 0.69 |
| 17 | (SEQ ID NO:33) | | | | | |
| | gagatataaaagtgccaatagtagc | | | | | |
| | (SEQ ID NO:34) | | | | | |
| Bavntr- | gatgacaaaagagccagatacaactccaatcgc | COMPLEX | ** | 281-348 | 4 | 0.21 |
| 28 | (SEQ ID NO:35) | | | | | |
| | gcttgtggcttacggctatgatgttttctttc | | | | | |
| | (SEQ ID NO:36) | | | | | |
| Bavntr- | gatgttcaatttaattgcacatgctcacgc | 3 | 4 TO 5 | 280-283 | 2 | 0.07 |
| 29 | (SEQ ID NO:37) | | | | | |
| | gtcactcctaacaccgagtatttttattggttttgtc | | | | | |
| | (SEQ ID NO:38) | | | | | |
| Bavntr- | gattttgtaggccgcgttggtgtagagac | 3 | 5 TO 6 | 180-183 | 2 | 0.18 |
| 27 | (SEQ ID NO:39) | | | | | |
| | gccatcttaaatgccttttcttcactttcagc | | | | | |
| | (SEQ ID NO:40) | | | | | |
| Bavntr- | gattttttctatgcaaaccaatctcctccttc | COMPLEX | ** | 796-1000 | 5 | 0.49 |
| 18 | (SEQ ID NO: 41) | | | | | |
| | gatctttttctggtttcgaaaaatcataaatactattacc | | | | | |
| | (SEQ ID NO:42) | | | | | |
| Bavntr- | gattttttgttgttgtttcatacgtctatccccttc | COMPLEX | ** | 296-424 | 5 | 0.52 |
| 21 | (SEQ ID NO:43) | | | | | |
| | gcctctctttttattttgtcatcttttgtcggtaagtc | | | | | |
| | (SEQ ID NO:44) | | | | | |
| Bavntr- | gcaaagaacggatttaggtatattgtattagcgattttac | 2 | 5 TO 6 | 347-349 | 2 | 0.27 |
| 20 | (SEQ ID NO:45) | | | | | |
| | gacgtattgttcctaatatttctttctcttcaatggcttc | | | | | |
| | (SEQ ID NO:46) | | | | | |
| Bavntr- | gcaatccctagctctgctgcttttgctttc | 12 | 2 TO 5 | 292-329 | 4 | 0.43 |
| 23 | (SEQ ID NO:47) | | | | | |
| | gattgctgcgagtgaaggggcgaataatac | | | | | |
| | (SEQ ID NO:48) | | | | | |
| Bavntr- | gcacagttgtatattgaaatagaaaagctatatgtaaagaaaatg | COMPLEX | ** | 439-509 | 6 | 0.33 |
| 25 | (SEQ ID NO:49) | | | | | |
| | ggaactccagacgcgaagaaagcc | | | | | |
| | (SEQ ID NO:50) | | | | | |
| Bavntr- | gcagtcaatatgtttccaaataaataagaagccac | | | 452-468 | | |
| 35 | (SEQ ID NO:51) | 5 | 6T09 | PE | 4 | 0.39 |
| | gatgcaagctggaaatgaattaggaaggattac | | | | | |
| | (SEQ ID NO:52) | | | | | |
| Bavntr- | gcctttactcatgaaacgataagaaggagcctac | 5 | 3 TO 5 | 131-141 | 3 | 0.49 |
| 24 | (SEQ ID NO:53) | | | | | |
| | gtatttgtttcgtaaatataaagccgaaaacacttgc | | | | | |
| | (SEQ ID NO:54) | | | | | |
| Bavntr- | gcggcttatcattgcgtacagtggataacac | 3 | 4 TO 6 | 406-412 | 3 | 0.52 |
| 19 | (SEQ ID NO:55) | | | | | |
| | gctctccccataaaacccgctaatgc | | | | | |
| | (SEQ ID NO:56) | | | | | |
| Bavntr- | gctatgcaaagcttggattatgggaaaaggc | 2 | 5 to 6 | 206-208 | 2 | 0.04 |
| 13 | (SEQ ID NO:57) | | | | | |
| | gtgtagctagctggactgtgaaatggactctc | | | | | |
| | (SEQ ID NO:58) | | | | | |
| Bavntr- | gctggatacttggatgaagaaattaactcctgaacaac | | | 496-556 | | |
| 16 | (SEQ ID NO: 59) | 8 | 8 to 15 | PE | 5 | 0.55 |
| | gcaaatagcgcgaaaaatccaagagcaagc | | | | | |
| | (SEQ ID NO:60) | | | | | |
| Bavntr- | ggatccaggagattatattttaaaggaagttcaagc | COMPLEX | ** | 434-606 | 9 | 0.76 |
| 32 | (SEQ ID NO:61) | | | | | |
| | gtcataaatcttaagcgtatccctaataaaacaagagcaaac | | | | | |
| | (SEQ ID NO:62) | | | | | |
| Bavntr- | ggatcgcagcttcaaaagatagt | COMPLEX | ** | 74-81 | 2 | 0.07 |
| 15 | (SEQ ID NO:63) | | | | | |
| | ggtaatagtaaacgctctaatgttcc | | | | | |
| | (SEQ ID NO:64) | | | | | |
| Bavntr- | ggtcaagtggttaagacaccgccctttc | | | 315-323 | | |
| 36 | (SEQ ID NO:65) | 4 | 4 TO 6 | PE | 3 | 0.04 |
| | gattcttctttcatctttacagcagcctcatatacattatc | | | | | |
| | (SEQ ID NO:66) | | | | | |
| Bavntr- | gtataacggaatccatatgaccaacctgtgtgaaatc | | | 347-355 | | |
| 33 | (SEQ ID NO:67) | 4 | 3 TO 5 | PE | 3 | 0.11 |
| | ggtatcagccataagcttaggccggctattc | | | | | |
| | (SEQ ID NO:68) | | | | | |
| Bavntr- | gtcaagtggttaagacaccgccctttcac | 4 | 3 TO 6 | 347-359 | 4 | 0.07 |
| 34 | (SEQ ID NO:69) | | | | | |
| | ggtccgtaacaagcccaacaacttgctc | | | | | |
| | (SEQ ID NO:70) | | | | | |
| Bavntr- | gtctatctaactacctcttattggctaaataaaataagcaagacac | | | 309-377 | | |
| 26 | (SEQ ID NO:71) | COMPLEX | ** | PE | 2 | 0.02 |
| | gtagatgaaacagcaaaacaagtgagtgaggc | | | | | |
| | (SEQ ID NO:72) | | | | | |
| Bavntr- | gttcatataattgcacctcatctagttttatttaaaaggaagc | 2 | 5 to 7 | 175-179 | 3 | 0.32 |
| 12 | (SEQ ID NO:73) | | | | | |
| | gattggaagatttcattacttacaggtgaagttcaaaaac | | | | | |
| | (SEQ ID NO:74) | | | | | |
| Bavntr- | gttgcatctaacaatgcagaacaaaaagatagc | | | 413-509 | | |
| 30 | (SEQ ID NO:75) | COMPLEX | ** | PE | 4 | 0.36 |
| | gttgtgtttgagtttgctgtgattttttctgttc | | | | | |
| | (SEQ ID NO:76) | | | | | |
| Bavntr- | tgtttgtgaccctgatttcat | | | 483-664 | | |
| 10 | (SEQ ID NO:77) | COMPLEX | ** | PE | 3 | 0.22 |
| | gtcaagctgaaggtaaacagaag | | | | | |
| | (SEQ ID NO:78) | | | | | |

*Arrays marked “COMPLEX” have mixed sequences that are tandemly repeated; that is, repeated regions are not exact repeats but mixtures of partially identical repeats.

Table 6 shows the DNA sequence from the additional variable regions of Bacillus anthracis; the sequence regions corresponding to the PCR primers listed in Table 5 are bracketed.

TABLE 6

(i) The VrrC3 locus (SEQ ID NO:79):
GAGCGACAATATGAAGAGAGTAAAAAGGAAGAAAAGTCAGTTGTCGATCAATG
GCTTGAGAAAAACGGTTATGAAATCGAACGCCAAGAGCCTATAGTAGAAGAAA
AAGAAGTGGTTCAAGAAATGAGTGCACCGCAGGAAGTTCCAGCGGCTGAATTA
CTTCATGAAACAATTGCTGAGCGCATGGAAGGTGCGAAGCAAGAAAGTGATGT
AGTGGACAAAAACATTCTACAAGAGGAATTAGTAGATTCTAAAGTAGAGCACGA
GGATACAATATTATCAGAAGAAATTAAACGTAGTACAGAAATAGAACAACCTAC
TATTGAAGTAGAAAAACAAGCACCAGAAGAATCAGTGATTGTCAAAGCAGAAG
CAGAAACAGAAGAGTCAGAAGAAGTAGAAGTAATTGCAGAPACAGAAGAGTCA
GAAGAAGTAGAAGTAATTGCAGAAACAGAAGAGTCAGAAGAAGTAGAAGTAAT
TGCAGAAACAGAAGAGTCAGAAGAAGTAGAAGTAATTGCGGAAACAGAAGAGT
CAGAAGAAGTAGAAGTAATTGCAGAAACAGAAGAGTCAGAAGAAGTAGAAGTA
ATTGCAGAAACAGAAGAGTTAGAAGAAGTGGAAGTAACTGCAGAAACAGAAGA
ATTAGAAGAAGTGGAAGTAGTTGCAGAAACAGAAGAGTTAGAAGAAGTGGAAG
TAATTGCAGAAACAGAAAAATTAGAAGAATTAGAAGAAGTGGAAGTAATTGCAG
AAACAGAAGAGTCAGAAGAAGTAGAAGTAATTGCAGAAACAGAAGCACCAGAA
GAAGTGGAACCTGTAGCACTTGAGGAAATGCAACAAGAAATGGTGTTAAATGA
AGCAATTGAACAAAAGAATGAATTCATACATGTTGCTGTGGCTGATGAACAAAC
GAAAAAAGATGTTCAAAGCTTTGCGGATGTTTTAATTGCGGAGGAACAATCAGT
TGTAGAAGAACACCGATTGTAGAAGAACAACCAGTTGCAGAAGAAGCACCGGT
CGTAGAAGAACAATCAGTTGTAGAAGAAACACCGATTGTAGAAGAACAACCAG
CTGCAGAAGAAGCACCGGTCGTAGAAGAACAATCAGTTGTAGAAGAAACACCG
ATTGTAGAAGAAGCACCGGTCGTAGAAGGACAATCAGTTGTAGAAGAAGCACC
GATTGCAGAAGAACAACCAGTTGCAGAGGAAACATCGGTCGTAGAAGAACAAC
CAGTTGCAGAGGAAACATCGATCGTAGAAGAACAACCAGTTGCAGAGGAAGC
ACCAGTCGTAGAAGAACAACCAGTTGTACAAAAAGAAGAACCAAAACGTGAGA
AAAAGCGTCACGTACCATTTAATGTTGTTATGTTGAAACAAGATAGAGCGCGAT
TAATGGAAA[GACATGCGTCTAGAACGAATGGAATGCAATCTTC]TATGAGTGAA
AGAGTAGAGAATAAGCCTGTACATCAAGTAGAAGAACAACCACAAGTGGAAGA
AAAGCCAATGCAACAAGTAGTAGTGGAGCCACAAGTGGAAGAGAAACAAATGC
AGCAAGTAGTGGAGCCACAAGTGGAAGAAAAACCAATGCAACAAGTAGTAGTG
GAACCACAAGTGGAAGAAAAACCAATGCAACAAGTAGTAGTGGAGCCACAAGT
GGAAGAAAAACCAATGCAGCAAGTAGTGGTAGAACCGCAAGTGGAAGAAAAA
CCAATGCAACAAGTAGTAGTGGAGCCACAAGTGGAAGAAAAACCAATGCAGCA
AGTAGTGGTAGAACCGCAAGTGGAAGAAAAACCAATGCAGCAAGTAGTGGTA
GAACCGCAAGTGGAAGAAAAACCAGTGCAGCAAGTAGTAGAGCCACAAGTGG
AAGAAGTGCAGCCAGTACAACAAGTAGTGGC[AGAGCAAGTACAAAAGCCAAT
TTCAAGTACGGAAG]TAGAAGAGAAAGCATATGTTGTAAATCAAAGAGAGAAT
(ii) The Bavntr-11 locus (SEQ ID NO:80):
TAATTGATTCATAAATCGTTTTAAATCATCTTGGTTCATAGAAAGTAAGTCTGTA
GTTTTCACTTTTAAATACACCTGCAAATTATTTCTATGATCGACTAAGATTCCTG
TTTTTTGTTCCAGTCGTTTATACGGAGCAATATCAACAATTGATGCATCCGTATT
TTTAAAAGAAGCAAAGATCGTTGAAATAAAACTTTGTTTTCCATTACTTTTCTCT
GACTCAATTTGTCTCTTTCGGTTTACCTTTTTAGCTTCAGCTGTCGCAACTTTCA
TCTTCATCTTTCCTTTCGTGAGATATAAAAAGGAGAGCGCTATATCTGCACTCT
CCTTTCATTCCTATTTCCTTTTTGTTTCGCTGCTGATGGTATTTACTTCGATTGG
ATGATAATTTTTATTGTCCATCTTCAACATATCCAAAATCACAAATATATHCTGT
TTGTAGGGCTGTTATGTGGTTTAATACATAGGAACAATCCAAACGTCCCAAACA
GCACATAAAACATAATCCTTCCAAACCCACCAACTGGAACTACCTTCGGCATAT
AACTTGCTAGAACTAACATTAGAGCTATAATCGCTCCATCTATGATATAGAAAAA
AGGCTAACTTAATTTCTTGTTTTGTATTTTTAGGATTACGCATCATTTCCATAATT
AATCATGCTCCTTCTTATCTTTCTGTTCCTTGCCCTTCCAAAAATCTCTGTCTAG
ATATTCAGCGTTTGTTACCGTTACCCACACAGGCTCTTTTTTCTTTCTTCTTTTA
AAAATCGTAAAAATCTGTTTAAAAAAAGTTGCGATTATGCTTAACACAATCGCAA
CTCCTAACATCAAGATAGCAAATATCCAGTTATTCATTTTAATATCCCTGCTGCT
TCTTCTCTATTTTTCTTGCTAACTTATTGTTCGAGTACAGATGGAGAGAAAGATA
AGTTAGAAAGGCAAAAGTAACAATGTAATGATATAAGTGACCTAACTCAAAACT
TATTTGAAAAACC[AGTATAGCAAAGGCAATAATGAAT]CCTGCTACCGTCATTA
GGATACTAGCTCGTAATTCTTGTTGCTTCATTTCAAAGTAAATTCTTCTTCGATT
CGCAATATCAATTACTTTCATGATTACCCCTCCTAATCTTATCTTCTACGCAATT
GATCTGTCCGATTTTGTGTACTTCCTCTTGGTCTTAATCAAGATTTTCTAACTTC
TTTCACATTATCTTGAACATTTACATCTCTTTGTACTGTAGTTGTCCCTACCCTT
ACATCCGCAGTTACTTCACTACTACCTCGAACGTTATTTTGAACAAATACATCAC
GCTGTACTGTCGTTGCTCCTGCTCTTACAGTATGATTTACTGCTTCACTTCCTC
TTACATTGTTCTGCACATTTACATCACGCTGCACTGTTGTTGAACCTGCTCTTA
CACTACTTTCTACTGCTTCGCTTCCTCTTACATTGTTTTGCACATTTACGTCACG
CTGTACTGTTGTTGAACCCGCTCTTACAGCACTTTCTACCGCTTCACTTCCCCT
TACATTGTTTTGTACATTTACATCACGCTGTACTGTTGTTGAACCTGCTCTTACA
TTACTTTCTACCGCTTCACTTCCCCTTACATTGTTTTGCACATTTACATCACGCT
GTACTGTTGTTGAACCTGCTCTTACATTGCTATCTACCGCTTCGCTTCCTCTTAT
ATTATTTTGTACATTTACGTCTCTCTGTATCGTTGCTACCCCTGCTTTTGCATCA
CTATTGATTGTTCCTGTCCCACCTGTACTTCTTAATGTATCTAGAACGTTCACTT
CACGCTGTACTGTTTGTGACC[CTGATTTCATATTTCCTTGCG]ATGCTTCAGTA
CCTCCCGTATTTCTTAATTTATTTTGTATTTCAATTTCTCTTTCCGCTGTTTGTCT
TTCGCCTTTTACATTGCCTTGCTGCCCTTCAGTACTTCCTGTATTTCTTAGTTTG
TTTTGTAATTCGATTTCTCGCTCTGCTGTTTGTCTTTCGCTTTTCACATTACCCT
GTTGCCCTTCTTCTTTTCCTGTATTTCTTAGTTTGTTTTGTAATTCAATTTCTCGT
TCTGCTGTTTGTCTTTCGCCTTTTATATTGCCCTGCTGCCTTTCATTAGAAATAG
AAGCTACATTCGGACGATTAGCTATTTTAGGGCTAGCAGATGGCTCTATACCA
GCATTTGCGTTATTTTGAATACCTTTACCTATTGTATTTAACGAATGACGATCAG
CGTTTTCTTGCGCCTTCTGTTTACCTTCAGCTTGACCGCCCTTAAATTCTGATA
CAGTTCCTTTCACACTGTCTGATACACCTTTTGCAACGCCTGATACTTTTTCTTT
TCCTTTATCAACTGCGCCGCCAACAGTCTCAGCCACCTTGTTTCCCGCATCGTT
CATCATGCCGCCAACTCCACGATTCGCAGCGTAACTCATGGCAGCACCCGTAT
TTCTAGGAAATTTCTTGGCCTGCATACCGCCGCCTTTGGCAGTATTCTTTTGTT
CGCCTTGTGATTCCGCACTCTCATGATTCGAATCCATTTCAGAGCGTTTAGGC
GTGTCTTCACGTGTTTGTCCACTTCCACCACTCATTAAGTTCTTCGCACCGCTA
AACATACGACCAGCGCCCTTTATAGCTCCTTTCGCTGCCATAGCTCCGCCAAC
CATACCCGTTAAGTGATTCTTTCCTTCTTTTAATCCAACATCAACACCAAAATAT
TTTAAGATAGATTCTGAACCATTAATTAATGCTACTGTTAAACAAACCATTGCTA
CGATATATAAAAATGCATTGATGTCTTTTTCTGCTAAATAATTAACGATAATCGT
GTAGACTCGTAAATTTAAACCCGTAAAAGCAAATACTAAAAATCCTTTAAAGATA
TCCTGTATAACCATTTTTGTTTTTTCGCCCGTTTCAATATGCGTAACGAACACAA
TTGGAGCAATAATTTTCTTCATGGCAATTTCAAAGATCGTCATAACAAATACAAA
AACAGTAAATAAATAGGCAACTCCTAGAGCTAATAACCCCCAAAATATAGTACC
GAAGTTCATTGGGTATCGAACATAGCCCCCTGGGAACTTATCTTTAAATGGATT
AAACACACTATCATTTATTTTTTCTACCGTTTCTTTGTCCCCATCGTTCGTAATTT
TATAAACCAAATATTCGGTTTCTTTAGGTATATCTTTTTGCGATGTCAACATTTC
TATAACTTTAGGTGTTACTACATCACCTAGTTGTGCTTTCAAGAAAATATCCTTA
C
(iii) The Bavntr-22 locus (SEQ ID NO:81):
TCCACAATCTTCCTCTCCTTGCACGAGCGGTGGCATATTATTATCCATCGGCAT
CATATTTGGATTTGGCATAGACGTATATTGCGGTCCAAACGGTGCTCCTTGCTG
ATATGGCATTTGATATGGATGTTGTTGTTGATAATACGGATTCGGCGGCATCAT
AGGCTGTTGATACGGCATTTGATATGGCACCATATTCGGTGGTTGATTGTTATC
CATAATTGGCATCATATTCGGCATTTGGTTGTTATCCATAATCGGCATCATATTT
GGCATTTGATTATTATCCATAATCGGCATCATATTTGGCATTTGATTATTATCCA
TAATTGGCATGATGTTTGGTGGTTGATTATTATCCATGATTGGCATAATGTTTG
GTGGTTGATTGTTATCCATGATTGGCATAATGTTTGGTGGTTGATTGTTATCCA
TGATTGGCATAATGTTTGGTGGTTGATTGTTATCCATGATTGGCATAATGTTTG
GTGGTTGATTATTGTCCATGATTGGCATAATGTTTGGCACTTGAATATTTGGCG
GTATTACTTGCGGAATCACTACATTTTCTTTCGATACATTTGGCGACACTATATT
TCCTACATTTTCTTTCTTCACTTGCGGTGATACTATATTCCCTACATTTTCTTTCT
TTACTTGCGGTGATACTATATTTCCTACGTTTTCTTTCTTTACTTGCGGTGATAC
TATATTTCCTACGTTTTCTTTCTTCACTTGCGGTGATACTATATTTCCTACGTTT
CTTTCTTCACTTGCGGTGATACTATGTTTCCTACATTTTCTTTCTTCACTTGTGG
TGATACTATATTCCCTACATTTTCTTTCTTTACTTGCGGTGATACTATATTATCTT
TTTTCGTTTGCGGAGAAATGATGTTATTAGGTTTCATTTTAGTAATTTGAGGAGC
AATTAATTCGCTTCCTTTTTTAATTACATCTGAAATTTTATATTCTTTTTTCGCTTT
TATTGGTGGTTGC[GGTGGCTGTGGTAAGACATTTACCGAAAAC]TTCGTGTGTTTT
CTTTCTCTGCTGGTTTTTGCTTTTCTATAACAGGTGGTTTTTTGGATAACAGACG
GTTTTTCAACTGGTTTTTCAACTGGTTTTTCTATTTGAATTAGCTTTTCTTTTTGC
ACTTCTTTTTGCGGTTTCACCTGCATTTCCTTCTGCGGTTTTACCTGTACTTCTT
TTTGTGGTTTCACTTGCATTTCCTTCTGTGGTTTTACTTGCACTTCTTTTTGTGTT
TGTTGCATAGCAGGCTGCTGTGTAATTGGCGCTGATTGATACGTAACTTCTTCT
TCATCTTCTATTCCAAGTGGAGTTGGAGTTGCTGCAAATTCTTTTTGTTGCACTT
CTTTTACGTATTGTTTTGGAGGTGCTGAGCCTGCACCAGCATGTTGTTTCACCT
GAACGCCGTTTGATGGTACTTTAATTTTCATACCTGGCATGATGAGATCCGGAT
TACTAAGTTGTGTATTTGTTT[GTTTCAGCGTATCAAAATCCACTCCGTACTTTTT
C]GCAATTTTCCAAAGGGTATCCCCTTTTTGCACGATATGAATTTTCAAATTTTC
CCCCTCCTGTATAACTTATCTATAAGTAACAAATCTCATGAAGTAACTCTTTCTA
CATAACGTTAACTTCTTTTCACTTTCATCACTTCTTCGATTTTCAATATCATTTGA
AGTGGAATAAGCATAAAGTGAAACTTTAATCAGTGGGGAGATTCATCCCCCAC
TGATTATTAGTTGAACCAATCGGACGTTTACGGGCAGTTGATCTCCGCCTAACT
TCTTTGTTCCAATCCAACTTTGAAGTATGAGATTTACTGCCCGTTAACGCGGGA
TAAAAAAATCACAATGATTTCTTCAAAACAATGTATGCCCATTTCGTTCTCGTTA
TGATTACATGTATGAAAAAAAGCAACGGGAATCCCGCTGCTTCACACACGCTC
TAACATACGATTTAATGCAAGAACCGCTTCTTCAGTTACTTGTTTATCTACGCTA
ATAACGTTAATTTCTTCTCCTCTTTCTATCGTTTCAAGTGCCCATAATAAGTGCG
GCAAATCAATTCGGTTCATCGTTAAACATGGACACATAAACGGATTAAGTGAAA
CAATTTCTTTATCTGGGTGAAGTTGAATAATTCGGTTCACTAAATTCATTTCTGT
ACCAATCGCCCATTTACTTCCGGATGGAGCCGATTCAATCATATCAATAATGTA
TTTCGTTGAACCTGCATAATCCGAAGCAGCTACAACTTCATAGCAACATTCCGG
ATGTACAATAATATTCATATCTGGATGATTTTTCCGTACATTCTCTATATTTTTCA
CTGTAAAGTTTTGGTGAACTGAACAATGCCCTTTCCATAAATCACTTGAATTTC
TTCTATATCTCCATCGTACTCTAATGAATCAGTATGTGGATCCCATACTGCCATT
TTATCTAACGGGATACCTAAGTCGTACGCTGTATTTCTCCCTAAATGTTGATCC
GGTAAAAAGACGAGACGCTCTT
(iv) The Bavntr-31 locus (SEQ ID NO:82):
TATACGATTGAAAGCTTAATGCATGATGGAAAAGCACTTCAAACAGGAACATCC
CATAACTTTGGAACGAATTTCTCTGAAGCATTTGATATTAAATTTTTAGATCGTA
ACGGTAAGTGGCAATATGTACACCAAACATCTTGGGGCGTATCAACAAGGATG
ATAGGTGGACTGATTATGGTTCATAGTGATAATAATGGGCTTGTAATGCCTCCT
AAAGTCGCACCAGTGCAAGTTGTTATTGTACCGATTGCGCAGCATAAAGAAGG
CGTTTTAGCGAAAGCAACAGAGTTACAAGGACATATTCAAAAGGTTGCACGAG
TAAAAATAGATGCTAGTAATAAAACACCAGGTTGGATTTAATGAGTATGAAA
TGAAAGGTATTCCAATTCGATTAGAAGTTGGTCCTAAAGATATTGAAAAGAATC
AAGTTGTACTCGTAAGAAGAGATACGAAAGAAAAAGAGTTTATATCAATGGATC
AATTAGAAGAACGCATTCCAGCACTACTTGAGGAAATTCATAACTCTTTATTTAA
TAAAGCAAAAGTATTTCGTGATGAAAATACGTATAGTGTGACGAATTTTGAAGA
GATGAAAAAATTGGCTGATGAAAAGCAAGGGTTTATTAAAGCAATGTGGTGTG
GAGAATTAGCTTGTGAAGAGAAACTAAAAGAAGAAGTTGCTTCTCGCTGTATG
CCTTTTGAACAAGAACATTTAGCAGAAGAATGTGTTTGTTGTGTAAACAAATGG
TGTATTGGGGAAAAGCGTATTAATGCTCTTTTGGATAGGAAGGATGAAAAGGA
CTTTGGGCTGCATAATAAATGCAATCCAAAGTCCTTTTTAGTTTTACAATTTGTT
TCATGTATTTTTAAGATGGAAGGATGAGTTTTTATTTTGCGTTTTTAGGGCGAAT
AAATAGTAACATACCAATTAGGAATGTTGAAAGTCCCATTAAAATAGATGCAGG
ATAATGTGTTGCAGTATTCGGTAACTTATC[GCCTTGTTTTTTCGCATTATCTTTT
ACATTACGACC]TTGAGAGCCATTTCCTCCGTGAGAACTGCTATCACCTTTAACA
CGATTTCCGCCTTGAGAGCCACTGCCACCAGAACCACTGCCACCAGAACTGTT
GCCACCCGAGCCATTGCCACCAGAACCGTTGCCGCCAGAACCATTTTCGCCC
GAGCCGCTGCTACCAGAACCGTTGCCGCCAGAACCATTTTCGCCCGAGCCGC
TGCTACCAGAACCGTTGCCGCCAGAACCATTTTCGCCCGAGCCGCTGCTACCA
GAACCGTTGCCACCAGAACCGTTGCCACCGGAACCATTGTCACCCGAGCCGC
TGCTACCAGAACCGTTGCCGCCAGAACCATTTTCGCCAGAACCGTTGCCACCA
GAACCGTTGCCACCGGAACCATTGTGACCCGAGCCATTGTCACCAGAACCGCT
ACCATCGGAACCATTGCCATCAGAACCACTATCGTCCTGAGAGCCACCGTTTG
CTGGTGGTTTATTATTATCTTTTGAGTTACCTTCTACGAAATAATGGTTATTTTGT
ACAATCGCGTTTGTTATTGGAGAGAAGTTTTCTGTTGCTGCATTACCGATATTT
AATCCACTAAAGAAAGATAAAGAAA[GAGTTGATGCTAAAATGAAAATTTTCGTC
ATTGATTTC]CTCCTGAAAAATTAATATTCCATCTAAAATACATCCCTTTGTAATA
TTTCATAGGAAAAAGAGTTTTATGCAAAAAAACTATATATAGAAAAAAGGTGGTG
TATTTCTAAAGAGAGAAAAGTGTTAGTGTAAATTGTTTATGAATAGTAGAGTGG
TGAGAATTTTACACTCATTACAGTAGAGGAAGTGAAAGCAGATGAAAGAATATA
TTGCATTTGATATTGGTGGTACACAAATTAAATATGGAATTGTTTCAGAAACAG
GAATAGTACTAAAGCACAAAACAGTTCCAACAGAAATTCATTTGGGGGGGGAA
CAAATTATTCAAAAACTCATTCTTTTATCAAAAAAATTAATGAGTGAACATACGA
TTTCGGGAATTGGTATTAGTACTGCGGGAATTGTTGACGTTAATAGAGGGGTT
GTGACGGGAGGGGCGGATCATATTCCGGGCTATAGTACGATTCCTATTATTAA
TAGATTGCAAGAGGTATTAAAAGTTCCCGTATCTATTGAAAATGATGTAATTG
TGCTGCGCTGGGAGAAAAGTGGAATGGTATTGGAAGAGAGAAAGAAAATTTCA
TTATGCTCACCCTTGGAACTGGTATTGGAGGAGCGATTTTTATAGATAGAGAGT
TATATAGAGGGCATTCATATAGTGCTGGTGAATGGGGAAACATGTTAATAGAA
GGAAAAACGTTTGAAGAGGTCGCTTCCATTTCAGGGTTAATTCATCTTGTACGA
AATTATAAAGGAAAAGGTAATTGGAATGGGAAAACGATTTTCGAACTGTATGAT
AAAGGCGACAGGGAAGTTACGCAAGCAGTTGAGGTTTTCTTTAAACATTTAGC
GATTGGAATTAGTAACCTTGCTTATATTTTTAATCCAGAAACAATTATTATTGGT
GGAGGAATTACTGATAGAGGAAATCAGTTTTTAAAAGAAGTAAAAGAAGAGGT
AGGAAGATACTTAAATAAAGAGAT
(v) The Bavntr-14 locus (SEQ ID NO:83):
GCTATCATTTTTTTCATCTTCTGCACCTCCACCACTGAATATACAAGGGCTTTGT
TACAGGAATGTGGCAAAAAATA[GAGCCAGCATAACGCTAGCTGTGTTTTG]GAT
ATTAAAAATAAATATATATATATGGACAACTATAGTCTACTTAATATATATGTAGT
ACAATAACCGTGTTTTTCATCCTCATTAATTATAAAACCAAAATCATTCAAAGCT
TTAACATGCATTTTTCAACTAAAAAATAAGTGTATATAATATACGCTAAAACTA
GCACTAAAATTACTAGCGCT[GTACCTTTCCAATTCATACCACCCGTCATATG]A
ACTGGACTACCCGTTTCAGCTCTGTTGAGCCCATCATGTAAAGAACCGGTAGG
ATTGTTTTTCAATTCACTTTGCCTTATACGTTCTCTTTTTTCTTCCGGTGACTCTT
TCTTCATATGATCCGTTCACCTCCAAATTAATACACTCTTTACTAAATTATACATT
CATATTTTTCTTTTATATGCAAACTTAAAAATATTTACAACATAAAGAAAGCTAGC
GATGCTAGCTCTCTTTATAATCCAATATGTTCTTTCTTATTTACAGTAATCCCTC
TATGATCAATTTCTAGCTCACACACTTTCATAGACGTAAATACTCTCGCTAATTG
CTCAGCAATTTCTTGACGTTTCTCATACGGCGTTAATATAAAAATAGATGGTCC
TGCACCGCTTAGCGCTGTACCGTATGCACCGAATTCTTTTGCACATTTACGAAT
AGATGGTAATAACGGTACAAGCTCTAAACGATACGGTTCGTGAAAATGATCCC
TTTCCATCATTTCCCCTACAACTTTCCATTTCTTTTGACATAAAGCAGCTACTAA
TACATTACTTATTGCACTAGCCTTAACAGCTTCATGAAACGGAAATACATCTGG
TAATACAGAACGACTTTCATCTGTATTTAGCTCTTCATTTGGAATAAGTGAAATT
ACACCTAATTCCTTACTTTCAATTCTCACAACCGATACATTTTTTCCATCAAGTG
CTCCGATTACAGTTCCTCCTAATATAGAAGCAGCAACATTATCAGGATGACCTT
CAAAATTTGTAGCAATTTGAACTTTTTGATCAATCGTTAAGTTCAATTTGCCTAG
TTGATTCGCAAGCTCTATCCCAGCTACAATTGCTGATGCACTACTTCCTAGACC
TCTTGTTAGCGGGATGTTACTAGTAACTTCTATTATATGGGGTGATAAAGAAGG
ACATACTVACATGCCGTGCTAACAATTAAATTTTTATCGTCTGTCGGAATTGAA
TCTTCAAAGGAATGGATCACTTGCCATT[ATCAGACTTCTCTTTTACTACTACAT
GTAAATACAATGACAAAGCTATTCCTACTGAATCAAATCCAGGTCCAACATTCG
CTGTACTAGCAGGAACACGAATACTTAATGGTATCACGACATAATCACCCCTTT
AATATGCTCTTTAATTTGTTCTATATT
(vi) The Bavntr-17 locus (SEQ ID NO:84):
TCATTTCGAGAACAATACACTATCAATACCTGAAGATACAATAATCAATCGTACA
ATCCAGTTAAAAGAAGAAGATAAACTACACTTATATAATTACTACAAAACGATTC
CTAAACCTGTACGTCCTAGATATCACAGTGATGATCCACTCTTTGTCGCTTTTC
ACTTTAGGTTGGGGACTTATGTTTGGAGTTATGACGATGATGCACCAAAATTTC
TAACGGAAATCTCCGTTCAAAAAATGATTCGTTTAGAGATAAAACGTGCTAATC
TTCGAAAGGGAATTTCAGCACAACACTTTCGAAATACTTTTATATTGCGGATGT
TCCAGCAGCTATGTAAGGATGAACATATTATGAAGCAAGTCGGGTTTAAAACTC
ACGAATCACTGAAAAGATATCGTAAGTATTATTATGCAAACTCTAACGAGAACA
AAGGTTAAAACTCTGTACTTGCTATATAAAAATTATATACCTACAGTTTTTTAGA
TAAATGTAAGATGGAGCTGTTAAGATGAAGAAAAATAAGGAGTCATATTCCATA
CACGTATACACTTTGCGAGACAAGACTACTAAATCTATTAATTAATCGAGTCGTGG
CGTTCATTAAGGGAAGAAATGCAAGTATTAGGCATAACGGATAGTGACATTTTC
CAAATCCAAATGATTGAAATAACCCCAAATAAAAAAGCGCCATAACGGTTGTTA
TAGTGCTTTTTTAACTTCGCTCTCTACTTATAAGAACAAACCGAGGGGAGCGGT
ATGATAATATCTTATTCTCTTGCATTACTTAATTATGAATTATTTTTACTATTTCTA
GTTAAATTATCTCTAATTTGCTCAAAACGTTCCTTACAAGAACCTACAAATATAT
CCATACCTATATATTTCTTATCGTCAGAATTTATAACTTTCTCGATTTCATTTTCA
CACTCTTTAATAGCCTGTTCAAGGTATTTTAAAGCTAATTCTGGGTTGTGTTTTA
AATACTCCGAACCCGATG[GAGTTAAACCATAAGGAATACACCTTTACTATATCT
TTAATTTCTTGTTCTTTCTCATATATTTTACCTTTTATTCTCTCTACCTGTTCTTCT
GTCCACCCTTTGTATGCACCTATATAATACGCTGGCGCTTCAATTCTCCCTGCT
TTCACTGCTTGATTCATCCCTTGCTTTGTCATCTCTAATTCCTCTGCTAAATCAG
CAGTTGAATATAATTTTTCCGTGCTCATATTAGCGCCTCCTTGTATTTTAGGTAA
ACAAATTTTCGTAATCCGTTTACCTTCGTTAAGTTAAATTTATTGTACCACTACA
TTTTACATAAGTAAACAGTTTTCGAATTTATATTTCATATCGTAAACTAAAATCAG
AATTTTTAAATTAAAATCGAATTTATTTACACATTTGCACATGTGTAAATGTGTAA
ATGTGTAAATGTGTACATTTTTAATACAGACTTGTAGCCAAAAATACAAAAGAAT
TAAATGTGACATGT[GCTACTATTGGCACTTTTATATCTC]TAGTCTTTTCGTAAC
TCCATGCATATAAACAACCATTTACAAAGTACGGGAAGAAGAACCAATCAAAAT
GCACGGCTGCAAAGATACCATTAGCTATGATAATTGCTGTTGTACGATTAATCC
GCTCAAATAGTGTTCCTATTCCTAAAGGCAGAAACTTTCTAAAAATACATTCCTC
AATTACAGGCGCTATTAATAGCGGGACAATTAAATAGAAGATATTCAACTCTAC
TTTTGCATTCACTTGTTCTGCATTGCCTGTATCAATTCCAAAATAGCCCTTTATA
TAGAAAAAAAAGAGACTGGACAGGATAGCAAGTCCAGTCGTCATTAACACCCA
TTTCCCCTTTTGTACTACTCTTTTTACATTCAACTTTCTTCTCCCCTTACTATGAT
TCTTGTTTATTTTTTAGCAAATTCAAATGTGATCTTTCCGCTAGAATCAAGCTTC
AGATACGCATCAAATTCTTTCCCTGATTTTCCTTTCATCCCTTTAATCAATCCTG
TTTTCTTACCTGCTAGTAATTTCTTAACGTTTGTTTCGCTAATTTTTTTGTTAAAA
ATTCGCTTGGAAGCGAAAACTTGCAACCTTCCTTATAGCCTGTACAACCGTAAA
ACGTTTTGCGATTCGCTATGTCTTTGCCACATTTCGGGCATTTCCCTACGGAAT
CCTTTGTTTTCATTTCTTGTACTTTGCTATCTATCTGAAGCTCACCAATTGACTT
CGTAGCATTTACGATCAGCTGCTTGGTGAATTCTCCTGTCTTCGCAACAAATAC
CTCTTTAGAGCCTTGTTTCTTCCCGATTTTACGCAAATACCCTTCCCATTGCGC
TGTCATTTCGGGCTTCGCTAATAATGTGCCGCCCACGGCTTCGCAAAGATTT
CACCTTTCGGTGTAACTGTCACTTTATTCTTTTTCACTTCTATATATTCTTGTTTT
TTGAGTGTTTCAA
(vii) The Bavntr-28 locus (SEQ ID NO:85):
AAGAGCTATACAAAATTGGTAAACATAAACGTGTTCGTATCTTACCAATTTACG
GTGGTCAAGATATTAACCGCCAAATTCGTGCTCTAAAAAAACACCCACACATTA
TTGTTGGTACGCCGGGTCGTATTTTAGATCATATTAACCGTAAAACACTTCGCT
TACAAAACGTAGAGACAGTTGTTCTTGACGAAGCGGATGAAATGTTAAACATG
GGCTTCATTGAAGATATTGAAGCAATTTTAACAGATGTGCCAGAAACACATCAA
ACATTACTATTCTCAGCGACAATGCCGGACCCAATTCGTCGTATTGCTGAGCG
TTTCATGACTGAGCCTCAACACATTAAAGTAAAAGCAAAAGAAGTAACAATGCC
AAACATTCAGCAGTTCTATTTAGAAGTGCAAGAAAAGAAAAAGTTTGACGTGTT
AACACGCTTACTAGATATTCAATCTCCAGAGCTTGCGATTGTGTTCGGTCGTAC
AAAGCGTCGTGTTGATGAATTATCAGAGGCATTAAACTTACGTGGTTATGCTGC
AGAAGGTATCCATGGTGATTTAACTCAGGCGAAACGTATGTCTGTATTACGTAA
ATTCAAAGAAGGTTCTATTGAAGTTCTTGTTGCAACAGACGTTGCTGCACGTGG
TCTTGATATTTCAGGCGTAACACACGTATATAACTTCGATATTCCACAAGATCC
AGAATCATATGTTCACCGTATTGGCCGTACTGGTCGTGCAGGTAAAAAAGGTA
TTGCAATGCTATTTGTAACACCACGTGAATCTGGACAATTAAAAAATATCGAGC
GTACAACAAAACGTAAAATGGACCGCATGGATGCACCGACACTTGACGAGCCA
CTAGAAGGTCAACAACGTTTAATCGCTGAAAAACTTCAAAGCACAATTGAAAAT
GAAAACTTAGCATACTACAAGCGTATTGCAGAAGAAATGTTAGAAGAGAACGA
CTCTGTAACAGTAGTAGCTGCTGCTTTAAAAAT[GATGACAAAAGAGCCAGATA
CAAGTCCAATCGC]TTTAACATCAGAACCACCAGTTGTTTCAAGAGGTGGCGGT
TCTAAAAAACGCGGCGGTAACGGAGGCGGATACCGTGATGGTAATCGTAATC
GTAGTCGTGATGGACGCGGTGGCGATGGTCGTAACCGTGACCGCAATCGTGA
TGGTCGTAATCGTGACGGTAACCGTGATCGCAATCGTGAAGGTAGCCGTGAT
GGTAACCGTGGTCGTCGTGGCGAAGGTCAAGGACGCCCAGGATCTTCAAACG
GACGCGGC[GAAAGAAAACATCATAGCCGTAAGCCACAAGC]TTAATAATAAAA
AAAGAGAATGCCCTTTGCCGGCATTCTCTTTTTTTATTTCTGTAATTGTACATAC
TACATAATAACTTGTGTAGAAATGAGGTGCCACATGCTTGTAAGGCTTGGGCAT
GTCGCGATGAGTGTACATTTGAAAAATGCATCTCCATCTCAAACGATGACGTAC
GCACAGTTTCAAAAAATAGATGATCGGGAAGCGGCAATTCGTAAACTTGAAAG
AATTGCTAATTCGAATTTGGAAAATTGCTTACGGTTATTAAAACATAATAAAGGC
CATGATATATCCTTTTTTCGACTTAGTTCTAAGTTGATTCCTTTAGCGAATCATG
AGGAGTTGTTAGAGTGGAATTATATTCGCCCTTTAAAAGAAAATTTAAAAGTGC
TAGGTGATTACGCTATTCGAATGAATATGCGTATTGATTTTCATCCAGATCATTT
TGTTGTACTCAATTCACCTGAGGAGAATATTTTTAAACAATCTGTAAAGACGTTA
CAGATGCATAGAAAATTATTAAAAGGTATGGGGATTGAACATAAGCAACGATGT
GTAATGCATGTTGGGGGCGGATATAAAGATAAAGAGCTTGCATTAGAGCGTTT
TATAGAGAATTGGTCTAATGTCCCAAGAGGTATTCAGGAAATGATTATGTTAGA
AAATGATGATACAACTTTTACGCTCGAGGATACATTATATTTAGGAGAAAAACT
GGACATTCCCGTTGTATTTGATTTACATCACCATATGATGAATCATGATCGAGA
AGATTGGCATGAAGATTGGGCGCGTGTTGTACATACGTGGGAATCGTCTTTGT
TACCAGTGAAAATGCACATTTCTAGTCCTAGAGAGGGAAAAGACCCGAGAGCA
CATGCGGACTTTATTGATGTAGATACTTTTTTATCTTTTTTAAAAAAGATAAAGG
GAAGTGTTCCGCAAATTGATTGTATGATTGAAGCGAAGAT
(viii) The Bavntr-29 locus (SEQ ID NO:86):
TAGAGCCGCAAAACTTCCTCGTATTGAAATTACAAAACCAAGCAGTGTAGTTGG
GAAGAATACGGTAAGTGCGATGCAATCTGGTATTCTTTATGGTTATGTTGGACA
AGTGGAAGGTATTGTTAAGCGCATGAAAGAGGAAGCTAAACAAGAACCGAAAG
TTATTGCAACAGGTGGATTGGCGAAATTAATTTCAGAAGAATCGAATGTGATTG
ATGTTGTAGATCCATTTTTAACATTAAAAGGTTTGTATATGTTATACGAGCGGAA
TGCAAATTTACAGCATGAGAAAGGTGAATAAATAAGTATGAAAGATTATTTAGT
AAAAGCGTTAGCGTTTGATGGAGAAGTGCGTGCGTATAGTGTACGTACTACAA
ACACGGTAAGTGAGGCGCAAAGACGTCATGATACATGGAGAACAGCTTCAGC
GGCGCTTGGTCGTTCTTTAACTGCAGGTACAATGATGGGGGCAATGTTAAAAG
GTGACCAAAAGCTAACAATTAAGGTAGAAGGAAATGGTCCGATTGGTCCTATC
TTAGTAGATGCTCATGCAAATGGAGATGTACGTGGTTATGTAACGAATCCACAT
GTTGACTTTGAAGGAACAGAACAAGGGAAATTACGTGTATATCAAGCGGTAGG
TACTGAAGGATTTGTAACGGTAATTAAAGATATTGGTATGCGTGAGCCGTTTAT
TGGTCAATCTCCAATTGTTTCAGGAGAATTAGGAGAAGACTTCACGTATTATTT
TGCAGTTTCTGAGCAGACGCCGTCTTCTGTTGGTGTTGGTGTCCTTGTAAATG
GAGACGATAGCATATTAGCAGCAGGTGGATTTATCCTTCAAATTATGCCGGGT
GCACAGGAAGAGACAATTTCATTCATTGAAGAGCGTTTGCAGAAGATTCCTCC
TGTATCAACATTGATCGAACAAGGGCTTTCTCCAGAAGAGTTACTGTATGCAGT
CCTTGGAGAAGACAAGGTAAAAGTATTAGAAACGATG[GATGTTCAATTTAATT
GCACATGCTCACGC]GAACGTATCGAGAGTGTGTTAATTAGTTTAGGTAAAACG
GAGTTAGAGCAAGTTCGAGAAGAAGAAGAAGAAACAGAAGTTCATTGTCATTT
CTGTAATGAGCGATACAAGTTCTCTAAAGAAGATATTACAAATTTAATTGAGAAT
TTGTAATAAGGGAATTTACCCTCTAAATACATTGACAAACAGGGAATTTTCTGA
CAAAATCTAAATATA[GACAAAACCAATAAAAATACTCGGTGTTAGGAGTGAC]A
GGAATGCGAGTGGCACAATCAGTTTCAGAATTAATCGGGAAAACGCCGATCGT
TAAGTTGAACCGCATCGTAGAATCAGACAGCGCAGATATATACTTAAAATTAGA
ATTTATGAATCCGGGGAGCAGCGTTAAAGATCGTATCGCATTAGCTATGATTGA
AGATGCTGAGAAAAAAGGATTATTAAAAGAAGGCGATACAATCATTGAGCCGA
CAAGTGGTAACACAGGAATTGGCTTAGCGATGGTAGCAGCTGCTAAAGGATAT
AAGGCAATTTTAGTAATGCCAGAAACAATGAGCGTTGAGCGTCGCAATTTATTA
CGTGCTTATGGCGCTGAATTAGTATTGACTCCAGGGCCTGAAGGAATGGGCG
GAGCAATTCGTCAAGCGACTGAATTAGCAAAAGAACATGGCTACTTTATACCG
CAACAGTTCAAAAATCAATGAAATCCAGAAATTCATCGTTTAACAACAGGTGCA
GAAATTGTTGAGCAAATGGGTGATCAATTAGATGCATTTATTGCAGGTATTGGT
ACAGGTGGAAGGATTACTGGTGCTGGTGAAGTGCTGAAGGAAGGATATAAAGA
TATTAAAATTTATGCAGTAGAACCTGCGGATTCACCGGTGTTATCTGGTGGAAA
ACCGGGTCCACATAAAATCCAAGGAATTGGGGCTGGATTTGTTCCAGAAACAT
TGGATGTAGAAGTATATGATGAAATCATTCAAGTGAAAACAGAGCAAGCGTTC
GAATACGCAAGAAGAGTGGCTAAAGAGGAAGGTATTTTTAGTTGGTATCTCTTC
AGGAGCGGTTATTTATGCAGCGACAGAAGTTGCGAAAAAGTTAGGTAAAGGGA
AAAAGGTACTTGTTATTATCCCAAGTAATGGTGAACGTTATTTAAGTACACCAC
TTTATCAATTTGAGTCTTAATTACATGTGATTGAAAAAGCATCCTTGA
(ix) The Bavntr-27 locus (SEQ ID NO:87):
ATGACATATATATAAGATGGCTTTCATTTTATAACACTCCACTTACTTGATACGT
ACTTTCTGTCTGCTTTTCAAACCAATGGATTTTTTCTCTAAAACGGACGACTTCT
CCAATTACAATCATGCTCGGATTTTGAATTTGTTCTTTTTTCGCAACATCTACAA
TTGTCCCTAACGTCCCAACAACTGTGCGCTGCATAGATGTAGTTCCCCGCTCA
ATAATAGCGGCAGGCGTACTTTTATCTTTTCCATGCTTCATCAGTTGTTCACATA
TATAGGGTAAATTGCTAACCCCCATATATACTGCTAAAGTCTCTACACCTTTCG
CTAAATTTCCCCATTTCACTTCCTCTGCAGCATCTTCTTTTCGATGCCCTGTCAC
AATAGCAAAGCTTGCACTTGCATCACGATGTGTCACTGGAATACCCGCATAAG
CTGGTGCAGCTATTCCAGCTGAAATACCAGGAACAATTTCAAATGGTACACCTT
GCTTTGCTAACGCTTCGGCCTCTTCTCCCCCTCTACCAAATACAAATGGATCCC
CACCCTTAAGTCTCGTTACAATTTTTCCTTTTTTCGCATATTTAATAAGAAACGT
GTTAATTGTCTCTTGCTTCATCGTGTGATAATTTGGTAGTTTTCCGCAATAAATT
AAATCCGCTTCAGGCTTTGCATAGGAAAGCAATTCTTTATTTACAAGACGGTCG
TACAAAATAACATCTGCCTTCTCAATACATTTCAATCCTTTCACAGTAATTAAAT
CTGGATCCCCAGGGCCCGCCCCAACGATATATACTTTTCCCATCATTTCTCCTC
ATTTCTACTATTAAAGTGAAACTTCCATCCTTATATATGAATGGAAGTTTTCATTT
CTTCATATTTCAATGTTCCTTACTCATGAATAACAAAGGCCGCGCTTTTTCGAAT
CTTCTTTTTTTCAAATAAAGCAATCTCCTTTACCTCTGGTAACGGTAAAAAATGT
TTTTCAAGCTCAATCTCC[GGCATGTTAAATGCCTTTTCTTCACTTTCAGC]AACA
ACAACAACAATTGGAACAATACTATTCTCTAATACAGCTTCAAATCGATATAAAT
CCATATTGCCCCTCCTAAGACGCTATTACTTCTTCTAACACGTTATTTAACGCTA
TTTGTAAT[GTCTCTACACCAACGCGGCCTACAAAATC]ATAAAACGTTTCAGCC
GGTAATTTATTTTCTTTGAAATAACTTATAAATGATGCGAGTACATCAGGTAGAT
CCTCACCATCTATTTTTCCTTTTAACTTTTGATTATAAGCACCACCATCTAACAA
CGTTCCACCTACATATATTTCAAATGCTTCAAGAATCCCTTTTTCGTTCGTTTTC
ATTTTCACACCTTGTAATCCAATGTCCGCAATTTGACGTTGACCACACGAATTC
GGACATCCTACCATATGAATACGAACCGGAACATCGAGTGCAATTTGTGTATCT
AAGTATTCGGCAATTTTTCGTAATCTTTCTTTCGTTTCTACTAAAGCAAGATTAC
AATATTCAATACCAGTACAAGAAACCGCGTGACCAATAAACGATTTCGGATTCG
CTGATATCGCTTCAAACAACGGCTCAGTTAGTAAGCCCGTTACATTTTCCGGC
GGAACATTCGGAATAATGAAGTTTTGCGAATTACATGTACGAATTTGTCCATTT
CCATATTGTTTTGCAATTCTTGCAATCTCAAACATTTCTTCTGCATGTAGGCGCC
CTACCGGCACATTAAAACCTACATATTTTAGTCCCTCTTGTTTTTGATCTTGGAC
ACCATAAAAGTATCCCGCATTCCAGCCTTTGAGAGCACTTTCCCCTTTACTTTG
TAATGGTCCTGTATACTCTATTAGTTTCTCTTTAAACTTCTCTGGTCCCCAGTCT
GCAACAAGAAATTTCAAACGAGCTAAATGACGTTTTTCGCGATATCCAAAATCA
CGGAATATCGTAGCAATCGCAATCGCTACCGCCTTCACTTCTTCTGGTAAAAC
GAATACATCTAATTCATCAGCTAAATATGGACGAGCTGATAACCCACCACCTAC
TTTTATATGAAAACCAACTTTTTTCTCACCATCTATCTCTTTCTTAGCAGGTGTA
AATGCAACACAGTTAATCTCTGC
(x) The Bavntr-18 locus (SEQ ID NO:88):
GCTTCTCTCAATAAGTCGATTGCGAGTTCTAAAAAGAGAGCTTCAATTAAAGGT
GGGAATGGTACTTGTGCCCTTGATAAAATTAAAGTTTCAAGTAAATCACTTGGA
ATGAGTTCATAATGATAATTTAAAACTGCAACGTATAACGGTGTAGCGCATATC
GAGAATAGAACGGCGATAAGGCGTAAAATCCTTGAAAATGTAGCGTATAGCCA
AGAGACGTTGTAGTCTTCTGGTGAAATGAAAAAATCGAAATAGGATACAGGTG
TTAATAAAACACTTGGAGAACCGTCCACAAAAATAGCGATTTTTCCATCGATTA
ATGCTTTTGTAACTCTATCAGTGCGTTCTGTATTGATATAAAGAGGAAAAATCG
ACTTTTCTCCCATCAATTCTTGTATATAAGCACTATCATTAATTTGATCATATTCA
AGAGCACGTAATGATTCCTCGAGAAAATCTACATTATCTTTTTCAGCGAGATTG
TCTAAATACATCATGACAACTTTTGTTTTGGAAAACTCACCGACAATCATTTCTT
TTGTTTGTAAATCTAAAACAGGAAGACGTTTTCGAACTAAATTAATATTTGTGTC
AATGTCCTCTACAAAACCTTCTTGCGGACCAATAACAGTCGATTCGTTTAAAGG
TGGAGTTGGTGCACGATAATTATCAATTGCAATATTTGCGAGCATACATTTTTG
ATCTTGTTGATTTACTTGAACAATCGCATGTCCCTTTAAAACCATGTCCTCAATT
TTTTGTAAATCGTTTGTAATTGTAATGCCGCTCATTGGAATATGTTCCTTCAGCT
CTTCTAGTGATGCGTTAGGTCGTTCTAAAAGAGTAGGCATTAAATACTTTTGCA
ATTTCTCACCATCAAGTGAAGGACGATAGTAAGAAATCCAATAAGGCATCGTTT
CATCGTCAGATGTATGATAATTCACAAAATCACTAGACTTTTTCAGTTTCTCTAT
TAAATCTTGTAAAGAATGAATAC[GATCTTTTTCTGGTTTCGAAAATCATAATA
CTATTACC]TTTAGAATTTTGTTGCTTATCCTGATCTGAGTTGTCTTGTTTAGAGT
GTTGCTGTTTATCTTGTGCTAAATCATCTTGCTTGGAGCTTTGCTGCGTATCCT
GGAATGAATCATCCTGATCGGAGTCGTCTTGCTTAGAATGATTCTGTTGCTCTT
GAGAAGATTCATCTTGTTTAGAGTGTTGCTGTTGCCCCTGATCGGAGTCGTCTT
GCTTAGAATGATTCTGTTGCTCTTGAGAAGATTCATCTTGTTTAGAGTGTTGCT
GTTGCCCCTGATCGGAGTCGTCTTGCTTAGAATGATTCTGTTGCTCTTGAGAA
GATTCATCTTGTTTAGAGTGTTGCTGTTGCCCCTGATCGGAGTCGTTCTGCTTA
GAATGATTCTGTTGCCCTTGATTCGAGTCGCCTTGCTTAGAGTGTTGCTGTTGC
CCCTGATCGGAGTTGCCTTGCTTAGAGTGTTGCTGTTGCCCCTGATCGGAGTC
GTCTTGCTTAGAATGATTCTGTTGCCCCTGAGAAGATTCATCTTGTTTAGCACT
TTGTTGCTGATTTTGATTCGATTGATTTTGCTGCTTATCTTGAGAAGATTCCTTG
TGTTCCTCTTTTTTTTGCTCGCTATTGTTACTTTTATTATGTTTCGTACTTCTAGT
CTGTTCTTTGTTGTCATCCTCTTGATTGTTAGGTTTTTGCTCCGAATTATCTGTT
TCGTTTTTCTCTGTTGTATTTAATTTTTTTTTCTTACGTAGCCAATTCCAAATCAT
ATGTATGCCCTCTTTGATTGTGTAATATAGTGCTATTTTGTGGCTAATTTTGTAT
ATTATTCATTTATTTATGAAAA[GAAGGAGGAGATTGGTTTGCATAGAAAAAATC]
TTTTAGTAAGCAGTTCATACTATGACGAGAAGAGGGCGAGTCTGTGCAAAATTG
TAAAAAAGGAGAAGTTTATTTTAAAGGAGTATACATTTGAAAATGGTAGGACAA
TTCCTGTTCAAATGGGGTATGAGACGTATGGTACTTTAAATAGAGAGAGATCCA
ATGTGATTTTGATTTGTCATTATTTTAGTGCAACTAGTCACGCGGCAGGAAAAT
ATACAGCGCATGATGAGGAGTCTGGCTGGTGGGATGGTTTAATTGGACCGGG
AAAAGCAATTGATACAAATCAATACTTTGTTATATGCACTGATAATCTTTGTAAT
GTGCAGGTGAAGAACCCTCATGTAATTACAACGGGGCCGAAATCAATTAATCC
GAAAACTGGAGATGAATATGCGATGGATTTTCCGGTTTTTACATTTCTTGATGT
AGCTCGTATGCAATGCGAGTTAATAAAAGATATGGGAATCGCAAGGTTACACG
CTGTAATGGGACCATCAGCTGGCGGGATGATTGCGCAGCAATGGGCTGTTCA
TTACCCTCATATGGTAGAGCGGATGATTGGTGTTATTACGAACCCACAAAATCC
AATTATTACGTCGGTAAATGTAGCGCAAAATGCAATTGAGGCTATCCGGCTGG
ATCCGAGTTGGAAGGGCGGGAAATATGGAGAAGAGCAGCCAATGAAGGGGCT
TCAGTTAGCAAATAGAATGATGTTTATGAACGCGTTTGATGAGCATTTTTATGA
AACAACCTACCCTCGTAATAGTATAGAAGTGGAACCGTATGAAAAGGTTTCTTC
ATTAACATCATTTGAGAAAGAGATAAACAAATTGACATATAGAAGTATAGAGTTA
GTAGATGCAAATTCGTGGATGTACACTGCGAAAGCAGTTTTATTACATGACATT
GCGCATGGATTTTCGTCTTTAGAAGAGGCGCTGTCTAATGTAGAAGCAAATGT
ACTTATGATTCCGTGTAAACAAGATTTGCTTCAGCCGTCACGTTA
(xi) The Bavntr-21 locus (SEQ ID NO:89):
TCATTAGGTTATGAATTTAAACAAGACACAGTGGCAGGTAATACATTCTACTTTT
TAAACACAAATGATGGATATGATGCGACTCGTATTTTAAACGAATCGTTACTAC
AATCGATGCGTGAAAAGATTTCTGGTGACATGGTTGTTGCTGTTCCTCACCAAG
ATGTATTAATTATTGCTGATATCGTCAACGAAATCGGTTATGATATTATTGCACA
AATGACAATGAAATTTTTTGCCGAAGGGCATGTTCCGATTACATCACTTTCATT
CGTATATGAAGATGGGGACTTTGAACCAATCHTATTTTAGCGAAGAATCGGAA
GAAGACAGATGGAAAAGAGAAAGGATGAACGAAGTGAACGTTTTTTACAACCT
TGAAGGAATTGGTGACACTCTAATTGTTGCCTTACAAGATATTACTTTAGAGAA
CCGTACTTTTGATCGCAAAGGAGATGTTGCTCGCGTATACGATCGTGAAAGCA
ACGAAACAGCAGGATTCAACATCTTTAATGCATCTTCTTATTTAGAAGTAAAAG
AAACAGGTAACCTTACGTTAACGAAAGAACTTGTAGAAAAAATTAACGAAATTT
TAGCGAAGAACGGCTTTGAAGAAACAGTAGAAGCAGATCTTTCTCCAAAATTTG
TTGTAGGCTATGTAGCTGAAAAAGAAAAACATCCAAATGCTGATAAGCTAAACA
TCTGTAAAGTAGAAATCGGTACAGAAACATTACAAATCGTATGTGGCGCACCAA
ACGTTGATGCAGGACAAAAAGTTGTCGTTGCAAAAATCGGCGCTGTAATGCCA
AGCGGTATGTTAATCAAGCCAGCTGAGCTTCGCGGAGTTCCTTCTGCTGGAAT
GATTTGTTCAGCACGTGAATTAGAGCTTCCAGATGCTCCACAAGAAAAAAGGTAT
TCTTGTATTAGAAGATAGCTTTGAAGTTGGACAAGAGTTTAAATTTTAATTGATA
TAAGAAAAGAGAGGGTGTCCTTTTGGACA[GCCTCTCTTTTTATTTTGTCATGTT
TTGTCGGTAAGTCGATA[TTCATTGTCGAATCGTTGATATATTGAGAGTTACGGT
AGATATATTTGAAAAATCGTTGATATATTGAGAGTTACGATAGATATATTTGAAA
AATCGTTGATATATTGAGAGTTACGGTAGATATATTTGAAAAATCGTTGATATAT
TGAGAGTTACGATAGATATATTTGAAAAATCGTTGATATATTGAGAGTTACGATA
GATATATTTGAAAAATCGTTGATATATTGCGAGTTGTGACAGATATATTCAAAAA
ATCATTCATATTATAAGGATGTCACCTTTT[TCACAAAATCTATATTGTATTTTAT
AAACAATCTAGTTAAAATTTAGAAAAATA[GAAGGGGATAGACGTATGAAACAAG
AACAAAAAATC]GCAGAACTTCTGAAACGAATTGAAACTTCAAAACAGCAAGATA
TCGAGTTAGGTACTTATGAGATATATTTGTTTAGTCAAAATGAATTAGAAAAAGG
ACAAATTGGTTATCGATATGATAAACATAAAAATTCATTAATAAGTAAAGAACAC
GGAAAATGGAAAGATGAATGGATTGTTATTGGATATGAAACAGATATGGGAGA
CCCAGTTTTCGTGAATATCGATGATGATGCGTATCCAGTGTATACGGCAGAGC
GTGGTACAGAAGTGTGGCAACCCGTTCATATTGGAAATATAGATGAGATTATAA
AACAATTATAAAAAACTTTGAGACCGGTGCAAAGTGTAACGGATAGTGTAGATG
AATATTGGAAGAGTAAAGCTTAAACGCAGTCTTACGTGTAAGGCTGCGTTTTTC
TATATGAAAACACTCGTAAATTAGAATATTATGGATAAATATGACATGCTTAATT
TTACCTGCACTTTTTGCCGAATGCTGATACAATAAAGGATGTTACTATTTAGTAG
AAAGAGTGGTAAGCATGTTAGATTGGATGAAAAAGCTGTTTAACAAAGAGGAA
GAACAAACAGCGATGAATAAAGAAGTACCAAAGCAAGTTGAAAGTCAGCCGAA
AATTCCTCGTGTAAACCACCACACTGAAGCAAGAGAAGCACAAATGGCAAGTA
GGAATGCAGGTAAATGCCGTTTTCCATTAGTACCGGATAATGGGTTCGATGAA
GAGGATGTTATAGAAACAGGGCATTTTGAAGAACAACCTGTTCAAGCTGTAAC
ATATGAAAATGAGCCTATTCAAAGAGGAATCAAAGTGGAAAGAAGTAGACGAC
AGTATGTGGAGAAGGTAGTTTCTACATATGAAGAACCGGAAATGCAATATGAA
CCGGAGCGAGAACCTGTCGTAAAGAAAGCATCTACACCAGCACAAGAAAGTAA
CCGTAGACCATTTCGTCCAACAGAAATGATTTCGCCAATTTACGG
(xii) The Bavntr-20 locus (SEQ ID NO:90):
CTTTCGGAAGAATTTCATCAGGATGTTTCCAGCCGCCGTGAATTAACGTAAATT
CTGTTTTATTATCCCCTAAGTCTTTCAAATGAAATGAAACAACCCAGCCATCTGT
ATCCCATGAGAAAGATAAATGATTTGGCTCATCGATTTCTAACACTTTACATGG
TGATGGTCCAAATGGTGATTGCACATGAAATTCATGTCCTACCTCTAATACAAA
ATCATTTGGCATAAACCATGAGGCAATCCCTTCTGCAGTGGATACTACATTCCA
CACCTTTTGAATAGACGCGTTAAAAACGATCGTTTGTTTAATATCAGTTAATGTA
TGTTGTTGTTCCATTTTCATTCTCCTTTATTCGGTCTCAAATATAACACCTTTTG
GTTTCATTTAGATAATATAACACCTTTTAGTTTCATGTCAACGCTAACTCTTTTTT
ACTGTTGAGAAATTCTCAACATTTTTTCAACAGGACAAACACCCTCTGCGCATT
TCCTCATAATATACTCACAAAGGCAACAAAAAATAAAAACTTCTCTCAACATGAA
ACTCCATCATTCATCTTGAAAGAAGCGCCTTACGTATATCATGCGTATTTCGTC
TCTCCTCCTTCCCTCAGGGACACTACGCGAGATATAAAAAATCAGAAGGAATAT
GCCCACCATTTGAGTCGGTAGTTATTTCTCTTCTCCTCAACATTTCCTTAGGAG
CGAAATGACTGCCGACTCACTTTTTTCAAACAAAAATAAAAAGGGGATGAAAGC
TATGTTCTATTGCATTAACTGCTCTGATATTCACCATGAAAAACATCCGAATGAT
AAAGTATTCAAAAACGGTTTTTATATTGATCCATTTTTAGGTGATCGTTATCACC
TTGGTATGTGTAAAGATGCCCAAAATCATGAGACAGGGGATCCTCTTTTAACGA
CAAAGAAATTAGGCGCAACCCAATCTATTATGAACGTATTACCGACACATGTTG
TCCCAACATAAAGCATAAAAAAGAGCTGGCTCATCCAGCTCTTTTTTATGCTTT
ATAAACGATCATTGCTGAAAAACAATAAATTTGATTTTCGTCATCGTTAATAGAA
ACACCGACTTGGTACTTAATGTCTACAAAGTTACTATCATCAATCTTTTTCAAAA
AAACATTTACAGCGTCTTCTAAGTCTTTTTCGTGACTTTCATCAAATACTTTTAC
ACGAATCATGTTAACACCATCCTAGATCGCAGCCCTCCGCCGGTTGATTATTG
CCACGGTGCGTATCTATGATGCGATAAAACTTTCCGTCTTCATATATATATCCG
TCTTCTAACACATATTGTTTACCTTCAGTATAGACATAAAACTTACCTATCATAA
ATTTACTCGTATACAATTCCAAATAATCTGGTCTAAATTTAGCTACCACTTTATTT
GTAATATCAATTTTTATTGTGCGCCCCACCTTCTCTCCCTCCTTT[GCAAAGAAC
GGATTTAGGTATATTGTATTAGCGATTTTAG]ATTTTATGCTTAAAAATAAGTGGA
TATGCCTCCTTTCTCCACCTGTACTATTTTTCTAAAAAAATCATCCATAAATAAA
AGCAAAAAATATATATATAAAAAGAGTTTTTACTTATTGACTCAAATATAATTATT
ATGGTATTATATAAAATTTGACATTTTCATTCACTTATGCTATCATTAAAGATGGT
TACCACATATGGAGGTGGATGATTTGTTTCAAATTGGTGATAAAATCGTTTATC
CAATGAACGGCGCAGGAGTCATC[GAAGCCATTGAAGAGAAAGAAATATTAGG
AACAATACGTC]AATATTGTGTAATACGTATCATTAGTAAAGATATGCAAGTAAT
GCTTCCGATGGATCAATTACAAAAATCAGGTATTCGTTATATCGTAGATAAAGG
TACGTTAGATGATATACTTCTTGAATTTCAAAACGGGGAATCAGATACATCACT
TTCCTGGAAACAAAGATATACAATGAATATGGAAAAAATGAAAAACGGCAATCT
ACAAGATAGTGCAGAAGTTGTTCGGGATTTACTTCGCCGCAATAAAGAGAGAG
CTTTAAATGCGAGCGAAAAACAAATGCTAGATAACGCCCGAAATTAATGATGTTCA
GAATATCTTCAAGATACAATGAATCACTAAATTTAAAATAGCTGGCCGCTATTTT
TTTTGTAAAAAAAGACGAAGAGTTTTATCTCTTCGTCCTTTTCTATTACCCACGT
CCAGCTTGAAACGATGGATATAACGTCATTCCGCCATCTGCAAATAGCGTAATT
CCAGTTACATAACTAGCCTCTGACGAAGCGAGCCAAGTTGCTACTGCTGCTAATT
TTCTTCTGGTTTTCCAATGTAGCCCATCGGTATCATACTTTCTACGTCAGCACG
TTTTTTAGGATCAGCAAACTTTTCTGCATTTATCGGCGTATTAATTGCACCTGGT
CCAATATTATTTACTCGAATACCTTTTGGTGCATATTCCATCGCTAACGTTTCTG
TCATTAGTTTCACACCGCCCTTGCTGGCTGCATAATGCACGAATAGCGGCCAC
GGAATTTTCTCATGAACACTCGACATGTTAATGATAGATCCTTTAATATCGTGTT
CTACAAAATATTTAATCGCTTCACGGCTTCCTAAAAAAGCACCTGTTAAATTTGT
ATTAATTACTTTATTCCAATCTTCAAGCGGCATTTCATGCGACGGTACCGCATTT
TCTATCCCTGCATTATTAATCATAACGTCAAGCGTACCAAACTCTTTCACAGCA
GATTGAATGAGGTTCACAACATCAGATTCAACCGTCACATCACCTTTTACAGCA
ATCGCTTCTCCGCCTACTTTTTTAATTTCTTCTAATACATCAT
(xiii) The Bavntr-23 locus (SEQ ID NO:91):
ATAATTTGTTAAACCAGACTTACGTTCCGCTTCTGTAAACTTCGATTTAAAGAAT
GTAGTTGCGAATGCAATTGCAAGCGGCGGTACCATACCACCAGCCATAACCGC
TGAGTGCACCCCAAAGTTCTGTGCTTCTATTGCAGCAATACCAAATGTAAATGC
TGCTTTATTAATTGGACCACCCATATCAATTGCCATCATCGCACCTAAAATAAG
ACCTAGTAATATAGCATTTGTACCGTTCAAACCATTTAACCATCCTGTTAACATT
TCATTCAATGCTACTACAGGCGGAATTACTACTTTTTGCATAACAACTCCTGTAA
TCAATAATCCAAAGACTGGATATAACAAAACAGGTTTAATACCTTCTAACTGTAC
TGGTAATCCTGAAAATAGTCTTTTTAGCCCTAAGACAACATATCCAGCTAAGAA
ACCAGCAAHAATCCACCTAAAAAGCCAGCATTCGCATGTGCCGCTAAAAATCC
CCCGACAACACCAGGCATAAAACGAGGACGATCAGCAATAGAACTCGCAATAA
ATCCTGCTAAAATTGGTACAAGGAATAAAAATGCCCCTGTTCCCCCTCCTCCAA
TAGACATGAACAATTCAGCTAAAGGGCCTTCTGCTTTTATACCACCAAAACGAAA
ACGCTAGCGCAATTAAAATCCCACCACCAACAACGAATGGAAGCATATTACTTA
CGCCGTTCATTAAATGCTTATAAATTCCTAATCCTTTTTCTTTCTCTGTACTTTCT
GTCTTTCCATCTTCTTTTATTCCTTTAAAGATCGGTGCATCCTGTTTTACAGCAC
GATTAAGAAGTTTTTCAGTTTTTCTAATCCGGTCAGCGACTGGCACTTGAATGA
CATGTTTACCAGCAAAACGATTCATTTCTACTTGTTTATCTGCCGCAACAATTAT
AGCTGTTGCGCGTTCAATATCCTCTTTCGTTAAACCGTTTTTTATACCTGTTGAT
CCATTCGTTTCAACTTTAATT[GCAATGCCTAGCTCTGCTGCTTTTGCTTTC]AAA
CTATCCGCAGCCATATATGTGTGAGCGATTCCAGT[GGACAAGCTGTAACAGC
TAATACGTATGGTTCATTCCCTTCTGGTTGTGCAACTTCTACCTCTTCTTCTTTT
TCATTTTCTTTTTCATTTTCTTTTTCATTTTCTTTTTCATCAAATAGACGAAGAAG
TTCCTCTTCATCCTTTGCTTCTAACAATTGCTTACGAAATCCTTCATCCATTAAT
AGTGTAGATAAGCGTGATAACGTTTCTAAATGC[GTATTATTCGCGGCTTCACTC
GGAGCAATC]ATAAAGAATAAATGCGCAGGCTGTCCGTCAAGCGATTCATAGTT
GAtACCGCTTACACTTCTACCAAAACAAATCGCTGGTTGCTTAACAGCTTTTGT
TTTCGCATGAGGTATAGCAATCCCTTCACCAATGCCAGTTGTACTTTGTGACTC
CCGCTTTAAAATAGCTTCTTTAAATTCAGCTTTACTATTTAAACGATTTGCCCCG
TTTAATTTCTCAACTAATTCATCTATGACAGCTTCTTTATTTGAAGCTGTCAAATT
CATAATAACTGTATCCCTTTTTAATAGTTCTGTAATTTTCATATGCTGCTTCCCC
CTATCGCTTAGTTACAATTACTTGCGACAATAATTCTTCTACTTTTTCCTTTTCAC
ATAAATCAGCTGAAAATGCTGTTGCACTCCCCGTTGCAACGCCATATTGAAATG
CCTTTTCAATATCTTTTGTCTGTTCATATTTACCTACAAATCCAGCAACGAGAGA
ATCTCCAGCCCCAACCGAATTAATTACAACACCTTTTGGAACTGTTGCTTCATA
TATACCTTCTGCCGTAAACAATAAAGCTCCATCTCCGGCCATTGATACGATAAC
GTGCTCTACACCTTGTTCAATTAATTTTCTTCCATACGGTAAAATATCTTCTGCT
GTTGAAATTTCTACTCCAAATAACTCACCAAGTTCATGATGATTTGGCTTTATTA
AAAATGGTTTATTTTTAACTACATGCTGGAAAGGAGTACCACTTGCATCTACTAG
TACACGAATACCTTTTTCAGCTCCGAACGCTGCGATTGATTCATAAAAAGTAGT
TGGAATAGACGCCGGTACACTTCCAGCTAGTACAACATAATCTCCCTTTTGCAT
ACTTTCAATTTGTTTCATTAATTGTTCAAATTGTTCATTTGTCACACTAGGTCCTT
GCCCATTTAATTCTGTTTCTTCTTGCCCTTTTATTTTCACATTAA
(xiv) The Bavntr-25 locus (SEQ ID NO:92):
ACTAGTAATCGCGGTAGCATTATCTGCGCAATGTACAGATGCACTTGTAAATAA
AGTGACGAAAAATTTATTTCAAAAATATAAAACACCAGAAGATTATTTAAGTGTT
TCTCTAGAAGAATTACAACAAGATATACGTTCTATTGGATTGTATAGAAATAAAG
CAAAAAATATTCAAAAATTGTGCAGAATGTTATTAGATGATTACAATGGGGAAG
TGCCGAAAGATCGAGATGAGCTTACGAAGTTACCAGGTGTAGGTCGGAAAACA
GCAAACGTAGTTGTTTCGGTAGCGTTTGGTATTCCGGCAATTGCTGTGGATAC
GCATGTAGAGAGAGTGAGTAAACGGTTAGCGATTTGTAGATGGAAAGATTCAG
TGTTAGAAGTAGAAAAGACATTAATGAAGAAAATTCCGATGGATGAATGGAGC
GTTACACATCACCGTATGATTTTCTTTGGACGTTATCACTGCAAGGCACAGCGA
CCACAATGCGAAGAGTGCCCTTTACTAGAAGTATGCCGAGAAGGAAAGAAGC
GAATGAAGGGGAAATAAAGGATGGAGCGAGTTATAGAAATACCGAGAGAATTT
CGGTGCTTACCATTTTTTAAAGAAAGTGTAAATTCGATTGCGTATTATGCAGAA
CAGTCTTTCGAAGAAACAATACAAAAGACTTATTTTATATATGATATTGAAAAAC
AATATGAGCCATGGAATGAAATTGAAAACAGTATTCCGGTAATGTTGAATGTAT
GGAAAAATAAGCATGGGGACATTGCTACTTTATTTCGAAACAGAAAGAAACAAG
AAGCTGAAGGTCCGATGATTCTTTTTGCAGCACATTTATTATCAATTGTATATTG
GCTAAATGAGCAACCTGTTCATAGTTTGAATAAAATAGAAGATTTTACGAGTGA
ATTGGAAGTGCAACCAGTTAATTTTATAGAGCGATATTCGTTCATTATAAAGAAA
CCGAATAATTATCATTCTTATATTCAGTTA[GCACAGTTGTATATTGAAATAGAAA
AGCTATATGTAAAGAAAATG]ATAACAAAAAAGAAGTCCTGTTCTCGTTAAGAGA
AAGGACTTCTTTTTTTGTATTAAGACTCTGTTGTAACAAAATTTCCGTCTGCGTG
AGGTGTATTTCCTCCGGTATTTTGTTGTTGTTCTTGCTGCTGTTGCTCTTGTTGT
TTTTTAGCTTCTTCTTCAGCTTTTTTTCTAGCTTCCTCTTCAGCTTTCTTTTGAGC
CTCTTCTTGCTGTTTCTTAGCTTCTTCTTCAGCTTTTTTTCTAGCTTCCTCTTCAG
CTTTCTTTTGAGCTTCTTCTTGCTGTTTACGTTGTTGTTCTTGTTGTTTCHAGCT
TCTTCTTCAGCTTTTTTAGTAGCTTCTTCTTGTTTTAGTTTATCTTCATTGGCCTT
TTTCTGAGCTTCCTCATCAGCTTTTTTCTTAGCTTCTTCTTCAGCCTTCTTCTTA
GCTTCTTCCTC[GGCTTTCTTCGCGTCTGGAGTTCC]GCCAGGCGCAGTGAAGG
ATGCTCCAACTGCAGGGCTTGTTCCAGTACCTTTTTTCGCTACTACAGAGAAGC
TGTAAGTAACGCCTGGTTTAATACCACCAAGAGTAGCTGTTGTACCTTTTACTG
ATAAACTACCACTTGAACCGTCAGTTGCTTTATAGCTTGCTGCATACGCATCAA
CTTCTGTTGGTCCAGACCAGTTTAGTGTAACTGTGCTAGGGCCATCGAAAGTG
ACATTAAGACCACTAGGTGCATCTACTTTAATTTGTTTAATTGCATCTTTCTTCG
CACCTTTTACATATAACTCTCCGTTTATTTCTTGTACAGAAGAAGGGCGTTCGA
AACGAGTTTTATCTGTAGCGAATTTGCTCATCATTACTTGGAACATTTGCTGTG
CAATTCTAGTATAACGATCACTAATGTAGTTTTCTGGGCCATTTTTTTCGTACCC
AGTCCATACTGCCATTGTATATTGCGGTGTATATCCAGCGAACCAGCTATCTCT
GTTTGCATCAGCTGGAATACCATATTCTTTTATAACGTCTGCATCAAAGTTTTGT
GTTCCTGTTTTACCGGCAACATCAACACCTGAAACGTATGCTGTTGGACCAGTA
CCGCCAGAACCAGGTTTTACGACATCACGAAGAACGTCAGTCACCATGTAAGC
TGTGTAGTCTTGCATCGCACGTTGTTCTTTCGGTTTAAAACTTTTCTTTTTCCCA
TCTGGGAAGGTAACTTCTTTTACGAAGTGCGGTTTATTGTAGTTCCCACCATTA
CCAAAGGCTGCATATGCACCCGCTAATTGTAATGGTGAACTATCGTTACTACCAA
ATTGCTGTTGATTCAAATGTTTTGCCATCTTTAAATGTCATACCTAAACCTTCAG
CAAACGCTTGTGACTTAGGAAGACCAACCGCTTGGGCTGTTTTTAAAGCTGGG
ATGTTTAATGACTTTTTCAAAGCTTCACGTAGTGAAACGTCACCTTTGTAACTAT
TTGT
(xv) The Bavntr-35 locus (SEQ ID NO:93):
TATACGCGCCCCATGTTTCTTGCACCAGGATTACCACTTCCTTCATCCCGAATA
TCAACGTTATGTCTCCATTTCGTTACTATATAA[GCAGTCAATATGTTTCCAAATA
AATAAGAAGCCAC]TAAATATAAAAACTGCATACTATTAATCATATAATCATTCAC
TTTCTGCATAGGAGTACTTATACAATATTCAATGACTATGTATATTGTACTTTTTT
ATCTACATCCCTTTATATATTTTAACATCATATTTTTACAAATCATACAAAAATTC
TCATGCTTCTATTTTTTAAAATATATATCCTTCATTAAATAATATGTTCCTTTTGC
TGCTGATTGTGATTGTGATTGTGATTGTGATACAAATGTAACTTCTCCTATACTC
TTCTCACAGTATTTCTTAAATTCAGCATTTATTTCAGGAACGAAACGTAATATGC
TGCCACTTACTGCAATTGAAGTTGATAAATCAAACTGCATCTTGTTATAAACATT
TACC[GTAATCCTTCCTAATTCATTTGCAGCTTGCATC]ATAATTTCATGTGCAGC
ATCATTCCCATTTCTAGCTTCCCGAATAACTAATGGTGCGATTGCTGCAACTTTA
TCTTTTGAAGAACTATAAACTAGTCTTTTTATATGAGACGATGTTAATAATTGAA
ACTCATCTTGAATCCTTA
(xvi) The Bavntr-24 locus (SEQ ID NO:94):
CTTGCCAGTGTGCTGGAAAGGGAATTGAAAATTCATCCATCTGTACTAGAGATA
ATATTTTTACTACTTAAGTATCATCATACAATAAAAAAATTCGGTTTGTTAAAATT
CTAAGT[GCCTTTACTCATGAAAGGATAAGAAGGAGCCTAG]AATCAGTAGGCTC
CTTCTTATGTTAATTTACTTCACTTCACTTCACTTCAACTCGCTGCCCTACA[GC
AAGTGTTTTCGGCTTTATATTTACGAAACAAATAC]TCACTACAATAAATAATAGT
CCGATGAATAAGCTAATAGTAATGGCTTCATGTAAGAAAATTGAACTTACAATA
ATAGCGATTAGAGGAATGAGAAATGTATAAGCCCCAACTTTACTTGCTTCACCA
GCTCCAACGAGTGTGAAGTATGCAAGCCACCCCATTGCAATAACAAAGAATGA
AATAAAGAGTAGTACACTAACAAACGGCATGCTCCAAGCGATACTAGACCAAC
TTTCAAACTCTGATCCAAATCCAATTAAGCAAAGTCCGCCAATAATAAGTTGCA
GTGTTACCATCCAAATGGAATTAACGCGGTGCCCAGTCTTCTTAATAAATACGG
TTCCAAGTGCCCAGCCAATAGCGCATCCTATCGCGAGAAGAATCCCGATAATA
GAAATATGTCCAGTTAAACTGCTAGAGCTAATAACACCTACGCCAATAAATCCG
AGTATAAGCCCGAAAATTTTTAACCCGTACATTGATTCTTCAAGCCATATCCAC
GAGAAAATACCAAGTAAAACGGGTTGTAGAAATACGATGGCGGAAAATAGACC
AGCAGGCATATATTGAAGGCCGACAGTTTGTAATCCGTAAAATATAATGATGTT
AAGTAAAGAAGAAATAACGTATAAATGCCAAGTTTCTTTTAAGTGTAACTCTTTG
TATTTCGGTAATGCGAACAGGAGTAAAATGAATCCTCCAATTAAAGTTCGAACG
CCTGCAAATAAAACGGGTGGTGTATAATGCAGGGCT
(xvii) The Bavntr-19 locus (SEQ ID NO:95):
GAGTAACCAGCGTTTTAGGCTATTATTCCAAAACTTGTTTTTTAATGTGGAGTCT
TCCTCGATGGAGGCTAAGCCTTCTACTTCGATATAATCTTCATCTAATTTTTTTC
CTTCACGCCCAAGTAGTACATGTACATTTGGATTGTTTTCGATATCTGTTATCTT
TTTGGATTGTCGATCTGTTGCAACATACAGTACAAAATCTTCGTGGAAAAACAT
CATAAAGGCACTGT[GCGGCTTATCATTGCGTACAGTGGATAACAC]ACCGGTT
CTTTGGCCTTGAATAATTGTTGTGATTTTTTCTTTTAAGTGCATATTTTTTTACCT
CTTTTCTTAATTTACATTTCCTCTATTTAAACAAGATGATTTAAACATCTTTCATA
CTGTTTTCTTCAATTCAATTTTTTAAACGTAATTGTGATGAAATCGGACAAGTTA
GGAGAAGAATAATAATAATATCGAGGAGAAATACATACGATGGAAGGATGGAA
AGCATGTTTAATAAAATATTTCTTATCGTGGCGCTTATAGGTGTACCACTGTCT
GTACTTGGGAAAACACTTCATTGGCCTCAAACAATTATGTTTGCTGTGTATTGT
ATTACGATTATC[GCATTAGCGGGTTTTATGGGGAGAGC]GACAGAAAGTTTGG
CAATTGTATCTGGTCCTAGAATAGGTGGATTATTAAATGCTACTTTCGGTAATG
CTGTTGAACTAATCATTTCAATTTTTGCACTTCAGGCAGGATTAATTGAGGTGG
TATTAGCTTCTTTAACGGGTTCTGTACTTGGAAATTTATTATTAGTAGGAGGGCT
ATCCTTCTTTATAGGAGGGCTTAAATACAAAAGACAAAGTTTTAATGTGTATGAT
GCAAGGCATAATTCAGCTTTATTAATCTTTGCTGTAGTAGTAGCATTCGTTATTC
CAGAGATTTTTTCAATGAAGATGGACGCGGGAAAGACGTATCAATTAAGTATC
GGTGTCTCGATTATTATGATTAT
(xviii) The Bavntr-13 locus (SEQ ID NO: 96):
TCCAGTAGTACCAGTGTCTCCAGTTGGCCCAGTTGGTCCAGTTGGTCCAGTTG
GCCCAGTTGGTCCAGTAGTACCAGTGTCTCCAGTTGGCCCAGTTGGTCCAGTT
GGCCCAGTCGGCCCAGTTGGTCCAGTTGGCCCAGTTGGTCCAGTAGTACCAG
TGTCTCCAGTTGGCCCAGTTGGTCCAGTTGGCCCAGTCGGCCCAGTTGGTCC
AGTAGTACCAGTGTCTCCAGTTGGCCCAGTTGGTCCAGTTGGCCCAGTCGGC
CCAGTTGGTCCAGTTGGCCCAGTCGGCCCAGTCGGTCCAGTCGGCCCAGTTG
GTCCGGTAGGAAGGGTAAATGGTGGTATCGGTGGTAATGTAGGTCCTACAAG
ATTAGGGTCAAATGCACTAGCTGATAAAGATTCATCGGGGTTTAATGCATTTGA
ATAATTATTATTTGACATAAATTCACCTCCATAAAGCGTTCATTATATAGTAGAT
GCAAAACCGAAAGAAAATGACACGGACATTTGAATTATTGAAAAGAAATCTTAA
ACTACTTGAACAATTTAAAAAAATGGAAAGTTTAGTATATGTATAACATATGATT
GATTTGGAAGAGGGTGATTATGTTGAACAAGCAAGGAATTACAATTAGTTTATG
TATGATTGTTCGAGATGAGGAGAAGACAATAGCCCGTTGTTTAGACTCAGTTG
AAAAAATTGTGGATGAAATTATAGTGGTTGATACAGGTTCCATCGATCGAACGA
AAGAAATCGTAGAGAAATACACTTCTAACATATATGATTTCCAGTGGATTGATG
ATTTTGCAGCAGCAAGAAATTTTTCTTTTTCAAAAGCGACGCAAGAGTATATATT
GTGGCTAGATGCAGATGACGTATTATTAGAAGATGCTCAAGAAGCATTGAAAC
TGTTAAAAAGAGAATTGGATCCTAAAATTGATGCTGTTTCGATGCCTTATCATCT
TGCAATGGATTCTAACGGGAAACCTTTATATTGTACAAAAAGAAATCGACTTGT
AAAGCGTGAAAAACAATTTCAATGGTTTGGAAAGGTCCATGAATATTTAGCTAT
TTCTGGTGAAACTTTTAGTAGTAATGTTGCTATTACACATAAAAAAGAAAAAAAG
GTAACAAATCGTAATTTGAAAATTTTTCAAGATGCTGTAGCAGCTGGAGAAGAA
TTGAGTCCGAGAGATTTATTTTATTACGCAAATGAATGTATGGATAACCAAAAAT
ATGATGATGCAGTTCTTCTATATGAAACATTTCTTAATCAAGACGAAGGATGGT
ATGAAGAGAAAATTTATGCGTGTGGTAAATTAGGAGATT[GCTATGCAAAGCTT
GGATTATGGGAAAAGGG]AGTTGAATCTTGTGTGAAGTCGTTTCGGTATGCTAT
TCCTAGAGGAGAGAATTGTACAAGAATAGGATATATATATATGGAACAACAAAA
ATATAATGAAGCGATATTTTGGTTTAAACTTGCAACGGAAGTACCAATGGCTAC
G[GAGAGTCCATTTCACAGTCCAGCTAGCTACAC]ATGGCTTCCCTATTTACAAA
TGTGCATTTGTTATAGCAAGTTAGGAGAACAAGACAAGGCTTATTATTACAACG
AATTAGCAGCTTCTTACGTTCCAAATAATGCTGCAATTGAATATAACCGCAAAT
ATTTTCGGAGCATTTTTGATCAACAAAAAAATCGTTATAGGTTGTTTTTTTAGAA
ATAGCTTATCAAGATTCTAACTCTAATAGATTGACTATATAGTAAAGTATCGGTA
ATGTAACCAGCAATGGTTATATTTATAGACTTCATATGAGGTTTATATTTCAAAT
TCAATTTGCATAAGGAGTGTGTGAAATGAATTCCCCAAAAAATTCTTTATATGAA
GAAAAATCTGGTCATTATTATAATGCAGTGAATCCGAATTTATTAAAGCATATAA
AAAAGGAATGGAAAGAAGTTCTTGATATCGGTTGCTCGAGCGGTGCGTTGGGA
GCAGCAATAAAAGAAAATGGCACACGTGTATCAGGAATCGAGGCATTCCCAGA
GGCAGCAGAACAAGCAAAAGAAAAATTAGATCATGTTGTTTTAGGTGATATAGA
AACAATGGAGATGCCATATGAGGAAGAGCAATTTGATTGTGTTATATTTGGTGA
CGTATTAGAGCATTTATTTGATCCATGGGCTGTAATAGAAAAAGTAAAACCGTA
TATAAAGCATAATGGTGTAATTTTGGCTAGTATACCGAACGTTGCTCACATCTC
AGTATTAGCTCCCTTACTTGCTGGAAATTGGTCGTATACAGAATATGGTTTATTA
GATAAAACGCATATTCGTTTTTTTACATTTAATGAAATGCTTCGAATGTTTTTAAA
AGCAGGATATTCTATTAGTAAAGTAGATCGTGTATATATTGATCATAAAATGTAT
GAACCGTTAATTGAGGAGTTATATGGAGTTTGTAAAAAATATCGTCTTGGAAGT
GGCTTCATGGCTGAAACGGTAGTATTTCAGTATATTATTGAAGCAGAAAAATCA
TCGTTATGAGCGCTGCTGAGAAAACGATATTATTTGTTACATGTATAAATGATT
CGAAAATGTATGCACAGTGTGTACGACATATATTGAGATTGGCAGTGCCACC
(xix) The Bavntr-16 locus (SEQ ID NO:97):
TAGAAAACTTAGACTTATTACCTTCTCATAGTGATTTTGAAAACTTTGAAAGTCT
TCTAACTTCAAAGTTCGGTCATGCTGAAACATCTGATCCTAACTATCATGAAATA
GAGACTAATAAAATTAACTATTTTAGAAACTTACTTGAACCCTTAAAACAAAATT
ACGACTTCATTATTATTGATAGCCCACCAACAGCATCTTACTATACAAAAAGTTC
TGCAATGGCTAGCGATTATGTTCTTGTTGCTTTTCAGACACAAAGTGATAGCCT
AGATGGTGCTAATGATTACATCACACGCTTTTTAGCAAGATTAGTAGAAGAATA
TAATGCTCAACTTGATGTTGTTGGAATTCTTCCAAATCAATTACATAGTGGAGGT
AAAATTGATACTACAGTATTACAAGATGCTAAAGATATTTTTGGTGAAAACAATT
TATTTAAAAACTTAATCCCTTACGCAAAACGAATCCAAAGCGTACCACGTATTG
GTTTAAATGTGGATCAGTATTGGGATAAAAAATTATTCAATGAAGTTTTGGAACC
ATTTACTGATGAATTTTTAGAACGAATTAATAAAATGGAGGAATTAAGAAATGAG
TAATGAACAAGGAAAAGGGATGGGATTTTTCGGGAACAAAGGAAAACCCGCCT
CTGAAAAGAAAGATGAGAAAAAGACGAAACTTGACCTTGACTATAAACCTGATC
TCAATCCAAGTACTCCTTACGATCCAACCCTACCTGTAAAAGCAACACTAGTAA
ATGGTGATAAACGTGGCACACTGCGTATTCCCTTAGAAACAAAACACGAGTTT
GATGCCCTTCTTGAAATTTCTGAATACCAATACACTTATGAATTGCTTGGTGAAA
T[GCTGGATACTTGGATGAAGAAATTAACTCCTGAACAAC]TTAGATTGTTCCAT
GCATCTCTTGAAAATATAAAACGCAAGGCAGCTATTAAGGAAGCTAAAAAGAAA
AAATAATTATATAATGTGTAAATGTGTACATTTACACATTTACACATTTACACATT
TACACATTTTTTATTCGAATCAACTTCAAAAAAACGAAATGACCCCCTGATATAA
CAACAAAACCCACTTAACTTTAATTTTTTTGTTTTTAATTTGTGTAAATGTGTACA
TTTACACATTTACACATTTACACAAATATGAAAAGGAGATATACCATGAGAACC
CTTATTATTATTACCTTAGTTGTCATAGGTTTAGTATTTTTTAACGTAATATCTTT
ACATACAATTGGGACATTAGCTGCCATAATCGGAGTTATAATAGCAATTGCGCT
TGGTGCTTATTTTTTATATCG[GCTTGCTCTTGGATTTTTCGCGCTATTTGC]AAC
TTTCATTGGCGTTATTTTAGTGATTTGCTTAATTACATACGCTGTAACTAATCTA
GTTTAAAAAGAGAAGTTAATTATGAATGCCCTAGAAGAAAATAAAAAACAAGTG
GGGAACAATGGGCTACAAAAACAGGCCGGATAGTCAATCTATTTAGAGTAAAA
GAAAATGGGTGCTGATACCTAACAGTGAACAAGAAGTTATATAGCGACACTCC
TCTTTGAATAGCCGCTGTTGATATTGTAAGACATAAAAGTAGAAACTTGATTTCA
AGAAAGAAATGCATATTCAAATTAAAAAATATTACATGCAGTAAGTAGGACAAG
AGCAACAGTATTGGAAAGTTCAAAAATAGAAGCTTCGAAAAGAAAATAAAAGGT
TATTGATTTCTTTAGAACCATACTTATATGAAAGCTTAGAGCAAATTGCATTAGA
GTTAATAACAGAAGGTCTAGACTATATTTATACGGAGGATTTGCCTGTTCAGCA
TTTTATCGAATTTAAATTAACATCATGCTATTGTAAGCCCTTATAATGATTAGCG
CTCAAACGGGGGTATTTAGAAAGAAAAATGGCATTACGTTTTATACTCAAAACA
AAGTAAAAGAATCCAACTATAAAAGAAGGATTCTTTCACCGGGTATTCGTTGAT
AAATGACTTCACTAAAGGAATTGAGTTTGATACACTCAGTTCCTAATTTTAGATT
ATAAAAAAGAAGATTATTTGTCAAAAAAATTTGCTATAGAATAATAAATGCTTTTT
ATTGAACTTAAATGAACACTCTCAGGAAGTATTTATATTTTTAATTTGCCTTTTTC
TCTTGAAAATTTCACCCTTACTTG
(xx) The Bavntr-32 locus (SEQ ID NO:98):
CGAAAAAATTAGAAAATGGTCAATTAAAATTATTGAAAAAAGATAGTGAATCTG
GTCAACTTCTACCAGGTGCAAAATTTGATGTTATCGATAAAGATGGGAAAGTTG
TGGAAACAATTGTTACAGATGATAAAGGTGAAGCTTTATCGAAACAACTTCCAG
TTGGAAGCTATACATTAAAAGAAGTAGAAGCACCGAAAGGATATGAATTATCAT
CTAGTTCAGTTTCTGTTGATGTAGAGGCTAATAAAGTAGTGACTGTAGATGTGG
TGAATAAAAAGATCCCCGAAAAAGTAACAGGTCAATTTGAAGTGGTGAAAGTA
GATGCAAATGATAAAACGAAATTGTTATCAGGTGCAGAATTCGAAGTGTATAAA
GATGGCAAAAAGGTAGCAGAACTGAAAACAGGTGAGAGTGGAAAAGTGATGT
CACCGAAATTACCGCTAGGTGAATACACAGTGAAAGAAACGAAAGCACCAGCG
GGCTACAAGCTTTCAGATAAAGAATGGAAAGTAACAATTCAAAAOGAGAAAGAA
GTAGTAAAAGTAGAGGCAGAAAACGAAAAAATCTTAGGTTCTCTACAAATTATT
AAAATGGATGATAAAGATCAAACGAAACGCTTAGCAGGCGCAGAATTTACATT
GAAAGATGTGAAAGGCAATGTTGTAAAAGAAGGAATTACAACAGATAAGTCTG
GAACTGTTAAAGTAGACGGACTTGTGCCGGGTGAATATACGTTAGAAGAAACA
AAAGCGCCAGAAGGTTATAAGGCATTAGAAGTAACAATCGAAGTAAACGTAGT
AGCAAACGAAGTAGTAAAACAAGACGTGTTGAATGAAAAAGTGAAAGAAGAAA
TTACAGGGCAATTAGAAATTACAAAGGTAGATGGTAATGATATAAATAAAAAATT
AGCAGGCGCAGTGTTTGAAATTTGGAAAGACGGAACAAAAATCGATACATTAA
CATCAGATGAAAATGGTAAAGCAATTTCGAAAGAACT[GGATCCAGGAGATTAT
ATTTTAAAGGAAGTTCAAGC]GCCAGAAGGTTATGAGTTATCTGATAAGGAAAT
CGAATTTACGATTTCTAATCAAAAATTTGAAGTTGTAAAACTTCAAATTACAAAT
AAAAAAGAAACAAGCAAAGGTCCAGAGAATCCAGGCGAAGAAACAGAAAAGC
GAGGTGAAGAAACAGAAAAGCCGGGTGAAGAAACAGAAAAGCCAGGCGAAGA
AACAGAAAAGCCAGGTGAAGAAACAGAAAAGCCAGGTGAAGAAACAGAAAAG
CCAGGCGAAGAAACAGAAAAGCCAGGTGAAGAAACAGAAAAGCCAGGTGAAG
AAACAGAAAAGCCAGGCGAAGAAACAGAAAAGCCAGGTGAAGAAACAGAAAA
GCCAGGCGGAGAGACAGAAAAACCGGGTAAAGAAACAGAGAAACCAGGTGAA
GGAATGGAAAACCCAGATAAAGAAAAAGAAAATCCTACTTTACCAGAAAAAGG
ACAAGGTACCTCTCATGCTCAACAGCTTCCAGCCACAGGACATGATATGAATTA
TCTTGCATTCATTGG[GTTTGCTCTTGTTTTATTAGGGATACGCTTAAGATTTATG
AC]TAAAAATAACTAATATGTTTTATAAAAGGCATAAGAGAAATATTATCTCTTAT
GCCTTTTGTTTTTTTATTTAAGCTTCAATAACTTTAAGCATTCGATTTAAAAATGC
GGCGGATTCAGCGCGTGTTGCTGTGCCTTTTGGTACGAATTCATTTCGTTCGT
TCCCTTTTACAATTCCATATGCATAAACGTTTTGTAATGCTTTTTTATCGTATGCT
AAGTGTTGGTCAGTGAATGGTAATTTCACTTCTTTTCTATAAATCCAGTTATATT
CTAGTGCACGGTCAATCATAATAACAGCTTCATCACGTGTTATTGTAGCATTAG
GATCAAATTTATTATTCCCACGACCATTAATGATACCTGCGCTAGCGGCGCGGT
TAATTCCATCTACTAAAGATGGATGTGCTTCGTTTAAATCTGTAAATTTAGCGTT
TCCAGCAGGTAAATTTAAAGCTCTCGACATTAAATTTGCAAACTCAGCTCTTGT
TACAAGACGGTTTGGCCAGTAAGAACCATTGCCGTCACCGAACATGATTTTTCT
AGCAGCCAATTGACGAATGTCTTGTTCATACCATCCACCTGTAATGTCGTCTTT
TTCATGAGGGACTTCTGGCGGCGGAATAACATCTTTATGATGTTCATTATAGTA
AGCGTTCGTACCTTTTAAGAAAGCATCCCATTGTCCGCGTTGAATCATAAATGC
AGGGCAGTTTTTTCCGCTCCAAAATTGATGCTWTTGCACGTGATCTAGAGATAT
GTTTAATTCATTCATTAAATAAGGAGCTAACTTTTTTGCGTTTTCTACCGCTTTC
GTATAATTCCCATCACTATTCACAGCAATTTCAATTCCGATAGATTCATAGTTTC
CAGTTTTATTTCCGGCATGCCAACCAACTTCATTTAATGGTAAATGTTGATA
(xxi) The Bavntr-15 locus (SEQ ID NO:99):
TCCTCTACTGTGAAAATTATCGGCAATGGTACACAAACAGGTTGGGCATCTATT
ATGGTTGCTATTACATTCTTTAGCGGTATTCAATTACTTGGTCTTGGAATCGTTG
GTCAGTATATCGCTCGTATTTATGACGAAAGTAAAAACCGTCCGATTTATATCG
TAAAAGAAACAATCAACATTGACCAAGAAGAGACTGCACTGAAAAAAGAAAAA
GTGAATGCATAATAAAAAAGGTAACAATGAAAACTATCATTGTTACCTTTTTATA
TTGTAATAGCCCATAAGTCTCAAACGAAATTCTTTTAAATTTGGATATACTTCTA
AATTAGACATTATGTTGTTATAAATAAAGGGGAGGAAGAACAGATGAAAATTAC
GTCTTTTGATTTTTGGCAAAAGTTCGGGAAAGCATTATTAGTTGTTGTAGCTGT
AATGCCAGCAGTTGGTTTAATGATTTCTATCGGTAAGTTAATTGGAATGTCTGC
TGGGGATATTAACGCAGTTCATACAATCGCTCGCGTAATGGAAGACATCGGTT
GGGCAATTATTACAAATCTACACATCTTATTCGCAGTAGCAATTGGGGGATCTT
GGGCGAAAGATCGCGCAGGTGGTGCATTTGCAGCGCTATTAGCATTCGTCTTA
ACGAATAGAATTACAGGAGCTATATTTGGCGTAAACGCTGAAATGTTAGCGGA
TTCAAAAGCGAAAGTTTCTTCAGTATTAGCAGGAGATTTAATTGTAAAAGATTAC
TTTACTTCTGTACTTGGTGCACCAGCATTAAACATGGGAGTTTTCGTAGGGATC
ATCACAGGTTTCTTAGGAGCTACTTTATATAACAAATATTATAACTATAACAAAC
TGCCACAGGCATTAGCATTCTTTAACGGAAAACGATTCGTACCATTCGTTGTAA
TTGTTTGGTCTACAGTAACTGCAATTGTATTATCACTTTTATGGCCATTCATCCA
AAGTGGATTAAATGAATTCGGTCGCT[GGATCGCAGCTTCAAAAGATAGTTGCA
CCAGTTGTTGCACCATTTGTATATTGGAACATTAGAGCGTTTACTATTACC]ATTC
GGTTTACACCATATGTTAACGATTCCGATGAACTATACAGAGCTAGGTGGAACA
TATACGATGTTAACTGGCTCGAAAATTGGACAAGTTGTAGCAGGACAGGATCC
TTTATGGCTTGCATGGATTACAGATTTAAACAACTTATTAGCAAATGGAGATAC
AAAAGCATATAACGATCTATTAAATAATGTTGTACCAGCTCGTTTCAAAGCGGG
ACAAGTTATCGGTTCAACAGCAGCATTAATGGGTATTGCTTTCGCGATGTTCCG
TAATGTTGATAAAGAAAAACGTGCAAAATATAAACCAATGTTCTTATCAGCAGC
ATTAGCAGTATTCTTAACAGGTGTAACAGAACCAATTGAATTCATGTTCATGTTT
ATTGCTCCAGTATTATATGTTGTATATGCAATTACAACAGGACTTGCATTCGCAT
TAGCAGATTTAATTAACTTGCGTGTTCACGCATTTGGATTTATTGAGTTAATTAC
TCGTACACCGATGATGGTGAATGCAGGACTAACTAGAGATTTAATCAATTTCGT
TATCGTTTCATTAGTATTCTTCGGTCTGAACTTTACATTATTCAATTTCTTAATTA
AAAAGTTCAACTTACCAACACCAGGACGTGCAGGTAACTATATTGATAACGAAG
ATGAGGCATCAGAAGGAACAGGAATGTACAAGAGGGTTCTTTAGCAACAAAG
GTTATTGATTTATTAGGTGGAAAAGAAAATATTGCTGACGTAGATGCTTGTATG
ACACGTTTACGTGTAACAGTAAAAGATTTAGATGTTGTTGCACCAGAAGCACAG
TGGAAACAAAATGGTGCTTTAGGACTTATCGTAAAAGATAAAGGTGTACAAGCA
GTATATGGTCCGAAAGCTGACGTATTAAAGTCAGACATTCAAGATATGTTAGGT
GCGTAATAATATGAAATTGCTAACTTTAAACTGTCACTCTTGGCAAGAAGAAAA
TCAAATAGAAAAGATAAAATATCTTGCTAAAGTCATTCAAGAAGAAGAG
(xxii) The Bavntr-36 locus (SEQ ID NO:100):
CTCCTCCATTTTTTAAATTTTATATTGTCGCGGGGTGGAGCAGCACGGTAGCTC
GTCGGGCTCATAACCCGAAGGTCGCAGGTTCAAATCCTGTCCCCGCAACCAAA
ATGGTCCCGTGGTGTAGTGGTTAACATGCCTGCCTGTCACGCAGGAGATCGC
CGGTTCGACCCCGGTCGGGACCGCCATTTATGTTGGCTCGGTAGCTCAGTCG
GTAGAGCAGAGGACTGAAAATCCTCGTGTCGGCGGTTCGATTCCGTCCCGAG
CCACCATTTTTTTATGCCGGCTTAGCTCAATTGGTAGAGCAACTGACTTGTAAT
CAGTAGGTTGGGGGTTCAAGTCCTCTAGCCGGCATCCTTTAGGGGCATAGTTT
AAAGGTAGAACTGAGGTCTCCAAAACCTCCAGTGTGGGTTCGATTCCTACTGC
CCCTGTAAAGTATATGGGCCTATAGCTCAGCTGGTTAGAGCGCACGCCTGATA
AGCGTGAGGTCGATGGTTCGAGTCCATTTAGACCCACCATATATTATTCCGCA
GTAGCTCAGTGGTAGAGCTATCGGCTGTTAACCGATCGGTCGTAGGTTCGAGT
CCTACCTGCGGAGCCATGGCCCGTT[GGTCAAGTGGTTAAGACACCGCCCTTT
C]ACGGCGGTAACACGGGTTCGAATCCCGTACGGGTCATAAAAAAAAGAGTCA
GCAATGTGCTGACTCTTTTTTATTGTGCGTGAATCCTCCTTTATCAACTGATTTT
CTTGAAATAAATAAATAAATAAAGGGATTTAGGAAAAGCTATGGTATGTATAAAA
AGGAGGAGACAAAATGACACGAGTGAGAGACGTTATGAGTACTCATATTGTAC
AGTGTACACCGCTA[GATAATGTATATGAGGCTGCTGTAAAGATGGAAGAA
TC]AATTGGATTGATTCCGGTTGTTGAAAATGAGCAAGTTGTTGGGCTTGTTAC
GGACCGAGATTTAGTTGTTCGAGGAATCTTTCCAGCACACTGCA
(xxiii) The Bavntr-33 locus (SEQ ID NO:101):
AACTGTCGTATCTAAGTTACTTAAAAATCCAATAATAAAACCATTCGCTTTATAA
TTTGCAGCTTGATTCCATGCACGAATATCTACTCCACCTTTTATAAATAAATCAT
TCATAAAGGTAGTAGGATAGTTACTATATCCATACAACATAAACCCAGATCCAC
ATATTAAAATAATCCTCATAATAATATGTGATTTTATAACTGGAATATACTTTCGT
ATATACGTAACTACAAGCACAATCAAAACAATGCCAACGATTGTCAGGAACATA
TTCCAAAAACTGAAATAATCCATAACCATGG[GTATAACGGAATCCATATGACCA
ACCTGTGTGAAATC]AGACGGATAGAATGGTTCTCCTCTAAAAATAAGCTTAAAA
TGGTTCACCATCATAAAAATAATTAAAACTAAGCTAGTTATCGTTGTACTTAAGA
AAACTCTCCAAACAAATTATATGCAAAAACGTATACCATATAAATAAATAAATA
ACAGACTTAATATATACTGCCCATATTGTAAATACACCCATGTATTTACTACTAT
AAGATTCATATCCTCCTGCACGATGATATACATCCAATTCATTGTATGAGCGAT
TAAAAATAATACGACATACACTC[GAATAGCCGGCCTAAGCTTATGGCTGATAC
C]TTTATTCAAATGAATGATGTATAGTAGTGCACTCCACAAAACAATAATGAGTA
TACTTAGCGGTATTTTCTCATTAAAAAAATCTTTCGTTTTCTCAAATTCGAAAGA
CCTTAATATAACAAAGAATAGCGTTTCTAAACAGATCGACCACATTCCACTAAG
AATGATAGAAATTAATAGATTCTTTCTCGGTTGCTGAGTAGAGGAAACTAAAATG
AAATTCATCTCTTCTTCGTTGCTGTGTAAAAACATTAAAACGCAATTTATTCCCT
CTTTCCTTTACCCCTTCGTCTCGCATCAACAAACATATTGTCATATACAAAATAA
CATATTTGTATAGAGTTAGTCTCCCTATGTGTTTGTATT
(xxiv) The Bavntr-34 locus (SEQ ID NO:102):
AGCACCTGGTTTGGGACCAGGGGGTCGCAGGTTCAAATCCTGTCTTCCCGATA
TTACCAAAAAACATGGGGCCTTAGCTCAGCTGGGAGAGCGCCTGCCTTGCAC
GCAGGAGGTCAGCGGTTCGATCCCGCTAGGCTCCACTTTATTTATTATATGTCT
CGGAGGTATACCCAAGTTCGGCTGAAGGGATCGGTCTTGAAAAACCGACAGGC
GGTGAGAATCGCGCGGGGGTTCGAATCCCTCTACCTCCTCCATTTTGTTTTTAA
GTGGTTTAGACATAAATAATAAAATATTTGTTTTTCCTTCATGGAGGTATACCCA
AGCTCGGCTGAAGGGATCGGTCTTGAAAACGGACAGGGGGCGAGAGTCGCG
CGAGGGTTCGAATCCCTCTACCTCCTCCATTTTTTAAATTTTATATTGTCGCGG
GGTGGAGCAGCACGGTAGCTCGTCGGGCTCATAACCCGAAGGTCGCAGGTTC
AAATCCTGTCCCCGCAACCAAAATGGTCCCGTGGTGTAGTGGTTAACATGCCT
GCCTGTCACGCAGGAGATCGCCGGTTCGACCCCGGTCGGGACCGCCATTTAT
GTTGGCTCGGTAGCTCAGTCGGTAGAGCAGAGGACTGAAAATCCTCGTGTCG
GCGGTTCGATTCCGTCCCGAGCCACCATTTTTTTATGCCGGCTTAGCTCAATT
GGTAGAGCAACTGACTTGTAATCAGTAGGTTGGGGGTTCAAGTCCTCTAGCCG
GCATCCTTTAGGGGCATAGTTTAAAGGTAGAACTGAGGTCTCCAAAACCTCCA
GTGTGGGTTCGATTCCTACTGCCCCTGTAAAGTATATGGGCGTATAGCTCAGC
TGGTTAGAGCGCACGCCTGATAAGCGTGAGGTCGATGGTTCGAGTCCATTTAG
ACCCACCATATATTATTCCGCAGTAGCTCAGTGGTAGAGCTATCGGCTGTTAAC
CGATCGGTCGTAGGTTCGAGTCCTACCTGCGGAGCCATGGCCCGTTG[GTCAA
GTGGTTAAGACACCGCGCTTTCAC]GGCGGTAACACGGGTTCGAATCCCGTAC
GGGTCATAAAAAAAAGAGTCAGCAATGTGCTGACTCTTTTTTATTGTGCGTGAA
TCCTCCTTTATCAACTGATTTTCTTGAAATAAATAAATAAATAAAGGGATTTAGG
AAAAGCTATGGTATGTATAAAAAGGAGGAGACAAAATGACACGAGTGAGAGAC
GTTATGAGTACTCATATTGTACAGTGTACACCGCTAGATAATGTATATGAGGCT
GCTGTAAAGATGAAAGAAGAATCAATTGGATTGATTCCGGTTGTTGAAAAT[GA
GCAAGTTGTTGGGCTTGTTACGGACC]GAGATTTAGTTGTTCGAGGAATTGCTG
AGAAACATCCTGGATCTAATAAAATTACAAATGTAATGACAACAAACATTATTTC
AGTTGCTCCAGATGATTCTCTTGAAAAAGCTACAGAGTTAATGGCCCAGCATCA
AATTAGACGATTACCAGTCGTTGAGAGTGATCAACTTGTTGGGATGTTAGCGC
TAGGTGATTTAGCTATAAGAGAATCAGCAGATGATCAAGCTGGATTTGCTTTAA
GTGAAATCTCGGAGCATACGGAATAAAGCAAGACTTTAATTAGTGGGATAAGTT
ACGTATAAAAATCAGATGCATTATACTAGTATATAGAATTAAGCTAATATGTTAG
CATGATGTATCTGAGACATTATATAATAAGTATACAGTTAGTAATTTCGACAGTT
TATTTAAATTTGTCATTCCTAATTTTAAATAAGAGAAAAAATGATATACTGATAGA
TATAAGTATTATGTTAGATTTCGAATATAACAATATTTATTATTAATGATGTAATG
ATGGATTAATATCTCCTATGTAGACAAAAGGGAAGGGGAATTATATGAAGGCT
GTACAGCGCGATCCAAATTGGAATTTGGTTACAGATAGATATATAGAGCCAAAT
AATTTCGCTGAATTATTTTCTTTGCTTGTACCTTGTCACCCAAAAGGTGAAGGG
AAAGAACGAACTATATTAGTGTGGAAAGAAAAAGAATTTTATAAAGAAGAAAAT
TTAGCGGCATTTATCGTATATGGAATGAATAAAGCAAAGAATTTACCGCAGTTT
CATAAAGATGAAATTCCAACTTTAGTACGTATTCTTCGCTTATGCCAAGAGATT
GGTTGGTATGAAGAAGCAAATACTTTTATGGTAAATCAAGGACTAGCTGAGTTT
GTTCACAGTTCATTGGAATATGAAACGTGGGATCTTTTGACGCAGGCGGTTGC
TTTAAACTATTTAATTATTAAATATCGTATTGGTG
(xxv) The Bavntr-26 locus (SEQ ID NO:103):
AGAAAGATAATAAAGATGATTTTGGTAAACATATTAAAGTAACATTCTTAAAAAA
TGTAGACAAGCATGAAACAATCGTAAAAGAAACAGCGCTTGATAAATTGAAGG
GTGACACACTTACTGCGGTAAATAACGATTTAGCTGCTTGGTTCTGGGATGAA
AAAGGTATTTCAGCAGGTAAATCTGATAAATTCAAAGTGAAATTTGAATTCGTT
GATAATAAAAAAGATCAAAATGAATTCCAAGGCGATAAGTTACAATTAACTTGG
ACGTTTGATGCACAGCAAGGCGATGGTGAAACAAAATAATAAGGACAACAAAA
AGAGACTATCAAAAACGATAGTCTCTTTTTCTGCTAAAAGTGAAATGTAAAAAG
AAACATAATTTTTATATCATTTTGTAATTCACTTTATTATAATAAAGATATAGTTCT
TGATAAAGAACGAGTTGTTCGATAGGAGAGATTAAAATGCTGAAGCCACCTCG
TAAATTCAAAAAGATGCTTATATTGCCATGTTTGTGTTCTATTACTTTTTATTTAG
GCTCTCAAATGATGACTTATACAGAAGCAGCTTTCATTCATGAAACGAAAGTAG
CGGCTACTATTTCTACAGCAAGTATTTTTCCAAAAACAGTTGATCAATTAATAGA
ACAAGCTAAGCAGCATAAGGAAGTTATTTTGCATGAGTATAAAGAGATGAAATC
AAAATTAAATGTTAGATCCACTCAGGAGATAGAACAGGCGCTTGTTATATGGAA
GCAAGGACGTGAGAAGATAGCTGCTGAGAGAGAATCGTTACAAAAAGTATATA
AATCAATAGAAGAACCTTATAATCAAGTACAAGAAGAGTTAAAAGGAAATACAA
CTGAATCGAATAAGCAAGTATTCTCGTATGTGAATGCTGGCTTCCATATAGTAA
AAGAAAACTGTCAGTATGTTGATAAAGAAGTGAATTTACAGGCGATCGATAAAC
AAATTCAAGATTTTGAGAAGTTACTA[GTAGATGAAACAGCAAAACAAGTGAGT
GAGGC]TGAGAAGCAAAAGGAACTAGAAGAGAAGAAACAAGAGGAAGTAAAGA
AACTAGAAGAGAAGAAACAAGAAGAAGCAAAAAAACTAGAAGAGAAGAAACAA
GAGGAAGCGAAAAAATTAGAAGAGAAGAAGAAACAAGAGGAAGCAAAAAATCA
AGAAGAGAGTAAGAAACAAGAAGAACAAAAAAATAAAGATAGCAAGTAAATTAT
AGTAACGTTCCAAGCAGATAGAGATTATAAAACAAAAAAGGAGAGAGAGCACTA
TCACTCACCTTCTTTTC[GTGTCTTGCTTATTTTATTTAGCCAATAAGAGGTAGTT
AGATAGAC]GAAGTTTATTAATTAGTGAAGATAGGTCAAAAATCAAAAATTTTTT
CTTTATTTTTCAATAATTTACTGAAATTTCTAAATAATATTGAGAAATAAAAATAA
CTGAAAGTATTAATAAATATGTGTTGTATTTATATAGGTATGTTCGTTATAATGA
ACATAAGGTTTTTAAAAAAGAACAAATATTAGCTAAGCTAATATAGCTTTAGTTA
TATCTCTTATTTTAAATAAGAGATAGTAATAAAAAATATAAAAAACAGCTAGGGG
GAATTGATTGTGAGTCTGAAAAAGAAATTAGGTATGGGAGTTGCATCAGCAGC
ATTGGGGTTATCTTTAATTGGTGGAGGAACATTTGCTTACTTTAGCGATAAAGA
AGTATCGAACAATACATTTGCAGCTGGGACGTTAGATCTTACATTAGACCCTAA
AACGCTTGTAGATATTAAAGATTTAAAACCAGGGGATTCTGTTAAGAAAGAGTT
CTTATTAAAGAATAGCGGTTCATTAACAATTAAAGACGTTAAACTAGCAACAAA
GTATACTGTGAAAGATGTAAAAGGTGATAATGCTGGTGAAGACTTTGGTAAGC
ACGTTAAAGTGAAATTCCTTTGGAACTGGGATAAACAAAGTGAGCCTGTATATG
AAACAACTTTAGCAGACTTACAAAAAACTGATCCAGATCTTTTAGCTCAAGACA
TTTTTGCTCCTGAGTGGGGGGAAAAGGGTGGATTAGAAGCTGGTACCGAGGA
TTATTTATGGGTACAATTTGAATTTGTAGATGATGGAAAAGACCAAAATATCTTC
CAAGGTGATTCATTGAATTTAGAATGGACATTCAATGCTAACCAAGTAAGCTGGA
GAAGAAAAATAATAAAAAAAGCGGGTATCCCCCGCTTTTTTTTATAAAGAAAAA
AGAAGTGCGGTTTGTAAGCACTTCTTTCTATTATTATTTTTGATTTTGCTTCCAT
TTTG
(xxvi) The Bavntr-12 locus (SEQ ID NO:104):
TAAACAGTTGTATCGGAAGAGAACTTTGCTTCTTTACTTAGTAGCCCATATTGC
ATAAGTTTTTCTTGAGTGGCAACGTTCATGTGTTGTAGCTCTGCCTGTAAGGAA
CGAATATCTTTCTTGTATTTACTACATGATTGCATCAAAATTTCTACACTTTCAG
CTGAACAAAAAACAAGGCGATTAACGTTAAAAATTTCAATGATTTGTTCAAGTG
TTAATGTATATTCTTCCTTTTTGAAAGTTGGAATTTGATATATTTCTGCACCAGC
TTCTTGTAACTTTTGCTTCATTACACTTGTTTTATTTGTTGCGGATGTAAATAAAA
CTTTTTTACCATGTAATGGTTTATGTTCTTTCCAAGCGATTTGATCTCGTAATGA
AACGACATCACCAACAATTGTCATAGATGGATTAGAAATATTTTCGTTTTTGAC
GATAGAAACAATGGTAGATAGTGTCCCTGTAACAACGCGCTGTTTACCAGTTG
TACCCCATTCAATGACTGCTACTGGCGTATCTTCTTTTTTTCCTGCTTGTCGTA
GGTTTTTACAAATTGTAGGTAAGTTTTTTATACCCATGTAGTAGGCGATCGTATC
GCTATTGTGAGATGAATTATATTTTCCATGATCGGTTAAAGAACCTTTTGCATGT
CCGGTTAGTAAAGTAACGCTATTACTGTAGTTACGGTGGGTAAGGGGGATACC
TGCATAGCTACTAGCGGCGGTGCTAGATGTAATGCCTGGTACAATTTCATATG
GAATATTTGCTGCTGCTAAAGTTTCTGCTTCTTCACCAACACGGCCAAAAATAG
ATGGATCTCCGCCTTTTAATCGGAGAACAATTTTTCCCTCTTGCGCAAATTGAA
GAAGGTGTGCGTTAATCATTTCTTGTCGCATAATATGATTCTTTGGCATTTTTCC
GCAATACATAAGTTCACATGTTTGTTTTGTATAGCTAAGAAAGAAAGGGTTTAG
TAAACGGTCATATAAAACAATATCTGCACGCTTTAAACACTCTATCGCTTTCTTT
GTAATAAGCCCTTCATCACCTGGTCCTGCACCAACTAAATATACATATCC[GTTG
ATATAATTGCACCTCATCTAGTTTTATTTAAAAGGAAGC]CAGGGGGGAGGCTT
CCTTTTGTAAATTACATATATATATATAAATGTCACCATTAATGACTTCTACTTCG
TACGTTTGAATACAACCGTCATCAG[GTTTTTGAACTTCACCTGTAAGTAATGAA
ATCTTCCAATC]ATGTAGCGGACAAAAGACGAATTCACCAGATACAATTCCTTC
GGCTAATGGTCCGTTTTTATGAGGACAACGATTTTCTACAGCTCGAATATCACC
ATTTGAAAGGCGGAATAGGGCGATAGACATACCTTTCATTTGGACCTCTTTACC
GATTTGAATAGGAAGATCTTCTGCACGCATAACTTTTATTTTATCTTTCGTTTGT
ATCATATAGATCACAGCTCCTTATTTCACAGTTTCTACTTCATACATCGCTTTTA
ATGATTTCGTTTCTAGTGCTTGTCCCCAAGCTTCTTTATATGTGCCACGGGCCG
TTTGGAAACGTTCATTTAACTTAGTGACCATATTTGCATCTTGCAGTATTTCTTT
TATGTGATCGAAGTCTAATCGTTCTGTCCAGTAGGCAGTACGCTCTCCGTAAAT
ACCAGTTTCACGATAATATTGCATGTAAGCTGTAGCGATGTGAAGAACATCATC
TTCAGTAGGGACAATCATTACAAAATCAGCTTCACGTACTTCTGTACCACCATT
TCCACCAATATAAAGTTGGTATCCATTTTCGACACAAACAACGCCAAAATCTTT
CGTAAGTACTTCCGCACAGTTACGCGGACAGCCCGTTACACCCATCTTCATTTT
ATGAGGTGTATCTACCATTTCTAATGATTGTTCAAGGAGCATACCAAGTCCTAA
TGAATCTTTCGTACCGAAACGGCAGAAACGAGAGCCAACACATGATTTTACATT
ACGAAGAGATTTTGAATACGCATATCCAGAAGTCATATTTAAGTCAGCCCATAC
GTTAGGTAAATCTTGTTTCTTAACACCGTATAAGCCAATTCGACTTGCACCGGT
AATTTTCACAAGTGGAACATCATACTTTTTCGCAACTTCAGCAATTTTCATTAAA
TCATCTGCTGTTGTAACGCCTCCGTACATACGTGGAATAACAGAGAATGTACCA
TCGTGCTGGATGTTACCATTCATTCTTTCATTAACGAAACGGGAGGCTTTATCA
TCTTCATATTCTTCTGGAATCGCCATACGTAAATAGTAGTTCAAAGCAGGACGA
CATTTTGAACAGCCATCTTCATGTACAAAACCAAGAACATTTCGTACTTCTTTTG
GTGATTTTAAGCCTTTTTCATGAATTGCCGCTACGACTTCATCACGTGATAAAG
GTGTACATCCGCACATACCAGCAGACTGGGCTGAGGCATCAAAAGCATCTCCG
AGTGTATGAGATAAAACTTGTTCTAC
(xxvii) The Bavntr-30 locus (SEQ ID NO:105):
TGGTATTTAACAGAGGACATTATAGAGCAGATGGCGAAGAATTTTTTGAGTAGG
AAATTATCCACAGGGAATTATTATGATTTAGATAGTATACAAATTTATATTCTATC
AATCTAAGTTCGCTTTTGAGTAGTTGGAAGTATATCTTTTATAGGAGGTTTTTAT
GGAATATGTTTATCATATGGTACCAGAAAAACTAAAAGGAGAATATTTAATCCC
ATTAAATGAGTTGAAAAGATTATATCCTAATTTATACGGTGATTACATACAAAAA
TATAAAGACCATCCTCAAAGAGAGAACTTATTAACACGTAAAATACCGAAACTA
GAATGTTTATGGAATGATGTTGTGCATTTTTTACCACTGCATCCTTATGAGATTT
ATAAAGCATTATTGGAAATAGGAGTAAACATCCATACAAATAAGCTATTTTATAA
AATTCCTATTAGCGCACTGAAGGGTCAGTGTATGGCAATATACAAGTACTCAAA
GCACAATTGGGGTGGACCAAATAGCGAATTAAAAGAATGTGAAATAGAATTCAT
CAATTTTAATGAATATAAAGAATTAAAGCAATTAAATACCTACACAAGAGAATAT
TATAGAGATGAATACAAAAATGGTAGGAGATTCGGGATGTTTCACTTAATTCCA
CATGTTCTTGTCAAAGGGAAAGTGGAGGTGAAACATATTGAAATCATAAATTGG
AGTATTGATCCAGTTACTGATTAATGTTACAAGCATTCAAAAATAAGATGCCATT
TTGAATTGATATTTGACTTAAAAGAATGGAAGAGTAATTAGATCTTATATGTAAG
AGTAAAAAAGAAGCTTACTAGAAGCTTCTTTTTTACTACTATTTTTCACATGCAA
CTCCATCGCCATCACGATCAAGATGTGAGTCATAACCTGGTTGTCCTTTATATA
GAGGAGCTTTCCCGGCAGCTCTAACTGCGGCACAATTTTTGTAATATGCACTA
CTAGTATTTCCTGAAGCAG[GTTGTGTTTGAGTTTGCTGTGATTTTTTCTGTTC]C
TCTTGTTGTTTGCGAGCTTGTTCATCAGCCAGACGTTTTTGCTCTTCTTGTTGTT
TACGAGCTTGTTCATCAGCCTGGCGTTTTTGCTCTTCTTGTTGTTTACGAGCTT
GTTCATCAGCCTGGCGTTTTTGTTCTTCTTGTTGTTTACGAGCCTGTTCATCAG
GCTGGCGTTTTTGTTCTTCTTGTTGTTTACGAGCCTGTTCATCAGCCTGGCGTT
TTTGTTCCTCTTGTTGTTTACGAGTTTGTTCATCAGCAAGACGTTTTTGTTCTTC
TTGTTGTTTACGAGCTTGTTCATCAGCCTGGCGTTTTTCATCCTCTTGCTTACG
AGCTTGTTCATCGGCTTGACGTTTCTCATCTTCTTGCTTTTGAGCTTTTTCGTCC
GCTTCTTTCTTTTCTAGTTCTTTTTTCTTCTC[GCTATCTTTTTGTTCTGCATTGTT
AGATGCAAC]TTGTTTACTAGTTGCTGTCGTTTTTTCAGAAGTAGGGTTTGAAAT
ACTTGATAACGCACCAAAAATAACAAATAAAATAGCAGTGATAATAAATTGTTTT
TTTGCTACACCTGTCTTTTTAATTACAGATAAGATTGCTAGTACGAGGAAGAAA
ACAAATAAAACAAAACTTAAAATAGAAAAAAATTCAACAATTGGGTTATTAGCAG
TCTCAGCACTAGTTATTATTAATACAATCGAAATCATAAAAAGAATAACGGCAG
GACGACCATACTGTTTTGCTTTTCCATTTTTCTTAAAGAATGAAATGATACATAG
AATTAAAAGAATAAAAGCAATTAAAAATAAAGCTGTACCAATATTACTCAAAATC
GCCATTTCTATACCTCCATTTGTAAGATTTTCATTGCTAATTATATAGGAATTTAT
TGGGTATTACACGATTTATATAAAATTAGTAGTTCTAATAGACTGATAAACATTT
ATTCGAATATATGAGAAAATGGATGTATTTCCTATTACATTTTTGAATAAAATCC
ATAAAGTATAATATACGGATTGAAAAGAGGAGATTTGAATGGAATTCCAATTGT
TGGTGACTTGTATATTACAAGAAGGTAATGCTTTCTTTTTAGTAACGAAAGTAG
ACGATGTTATTACATTGAAAGTACCAATTACAGCGGGAGTAGCAGGTTTATTCT
TAGCTTTAGGTGTACCAAGATGTTCTTAATTTAAATCTCTAGAGTAGAAAGAGG
AGGACAAGCCTCTTTCTACTAATACATAATTATTTTATGTAAACTAAAGTATTCTT
TTTGTAATAGCACGTTTAAAATGGACATGCATATACTATAATGTTTCATTGGATG
TTTGTGATAAAAGGTAGTTTTAAATAGTACATTCAGTGATATTTTTTTAGCTATG
AAAATAAAAGAGGTGCTATTATATATGAAAAGAA
(xxviii) The Bavntr-10 locus (SEQ ID NO:106):
TCGCAACTCCTAACATCAAGATAGCAAATATCCAGTTATTCATTTTAATAT
CCCTGCTGCTTCTTCTCTATTTTTCTTGCTAACTTATTGTTCGAGTACAGATGGA
GAGAAAGATAAGTTAGAAAGGCAAAAGTAACAATGTAATGATATAAGTGACCTA
ACTCAAAACTTATTTGAAAAACCAGTATAGCAAAGGCAATAATGAATCCTGCTA
CCGTCATTAGGATACTAGCTCGTAATTCTTGTTGCTTCATTTCAAAGTAAATTCT
TCTTCGATTCGCAATATCAATTACTTTCATGATTACCCCTCCTAATCTTATCTTC
TACGCAATTGATCTGTCCGATTTTGTGTACTTCCTCTTGGTCTTAATCTTGATTT
TCTAACTTCTTTCACATTATCTTGAACATTTACATCTCTTTGTACTGTAGTTGTCC
CTACCCTTACATCCGCAGTTACTTCACTACTACCTCGAACGTTATTTTGAACATT
TACATCACGCTGTACTGTCGTTGCTCCTGCTCTTACAGTATGATTTACTGCTTC
ACTTCCTCTTACATTGTTCTGCACATTTACATCACGCTGCACTGTTGTTGAACCT
GCTCTTACACTACTTTCTACTGCTTCGCTTCCTCTTACATTGTTTTGCACATTTA
CGTCACGCTGTACTGTTGTTGAACCCGCTCTTACAGCACTTTCTACCGCTTCAC
TTCCCCTTACATTGTTTTGTACATTTACATCACGCTGTACTGTTGTTGAACCTGC
TCTTACATTACTTTCTACCGCTTCACTTCCCCTTACATTGTTTTGCACATTTACAT
CACGCTGTACTGTTGTTGAACCTGCTCTTACATTGCTATCTACCGCTTCGCTTC
CTCTTATATTATTTTGTACATTTACGTCTCTCTGTATCGTTGCTACCCCTGCTTTT
GCATCACTATTGATTGTTCCTGTCCCACCTGTACTTCTTAATGTATCTAGAACGT
TCACTTCACGCTGTAC[TGTTTGTGACCCTGATTTCAT]ATTTCCTTGCGATGCTT
CAGTACCTCCCGTATTTCTTAATTTATTTTGTATTTCAATTTCTCTTTCCGCTGTT
TGTCTTTCGCCTTTTACATTGCCTTGCTGCCCTTCAGTACTTCCTGTATTTCTTA
GTTTGTTTTGTAATTCGATTTCTCGCTCTGCTGTTTGTCTTTCGCTTTTCACATT
ACCCTGTTGCCCTTCTTCTTTTCCTGTATTTCTTAGTTTGTTTTGTAATTCAATTT
CTCGTTCTGCTGTTTGTCTTTCGCCTTTEATATTGCCCTGCTGCCTTTCATTAGA
AATAGAAGCTACATTCGGACGATTAGCTATTTTAGGGCTAGCAGATGGCTCTAT
ACCAGCATTTGCGTTATTTTGAATACGTTTACCTATTGTATTTAACGTTTGACGA
TCAGCGTTTTCTTGCGC[CTTCTGTTTACCTTCAGCTTGAC]CGCCCTTAAATTCT
GATACAGTTCCTTTCACACTGTCTGATACACCTTTTGCAACGCCTGATACTTTTT
CTTTTCCTTTATCAACTGCGCCGCCAACAGTCTCAGCCACCTTGTTTCCCGCAT
CGTTCATCATGCCGCCAACTCCACGATTCGCAGCGTAACTCATGGCAGCACCC
GTATTTCTAGCAAATTTCTTCGCCTGCATACCGCCGCCTTTGGCAGTATTCTTT
TGTTCGCCTTGTGATTCCGCACTCTCATGATTCGAATCCATTTCAGAGCGTTTA
GGCGTGTCTTCACGTGTTTGTCCACTTCCACCACTCATTAAGTTCTTCGCACCG
CTAAACATACGACCAGCGCCCTTTATAGCTCCTTTCGCTGCCATAGCTCCGCC
AACCATACCCGTTAAGTGATTCTTTCCTTCTTTTAATCCAACATCAACACCAAAA
TATTTTAAGATAGATTCTGAACCATTAATTAATGCTACTGTTAAACAAACCATTG
CTACGATATATAAAAATGCATTGATGTCTTTTTCTGCTAAATAATTAACGATAAT
CGTGTAGACTCGTAAATTTAAACCCGTAAAAGCAAATACTAAAAATCCTTTAAA
GATATCCTGTATAACCATTTTTGTTTTTTCGCCCGTTTCAATATCCGTAACGAAC
ACAATTGGAGCAATAATTTTCTTCATGGCAATTTCAAAGATCGTCATAACAAATA
CAAAAACAGTAAATAAATAGGCAACTCCTAGAGCTAATAACCCCCAAAATATAG
TACCGAAGTTCATTGGGTATCGAACATAGCCCCCTGGGAACTTATCTTTAAATG
GATTAAACACACTATCATTTATTTTTTCTACCGTTTCTTTGTCCCCATCGTTCGT
AATTTTATAAACCAAATATTCGGTTTCTTTAGGTATATCTTTTTGCGATGTCAAC
ATTTCTATAACTTTAGGTGTTACTACATCACCTAGTTGTGCTTTCAAGAAAATAT
CCTTAC

The foregoing description of the invention has been presented for purposes of illustration and description and is not intended to be exhaustive or to limit the invention to the precise form disclosed, and obviously many modifications and variations are possible in light of the above teaching. The embodiments were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

What is claimed is:

1. An isolated nucleic acid comprising the nucleotide sequence of SEQ ID NO: 1.

2. The isolated nucleic acid as described in claim 1, wherein said nucleotide sequence is used for identifying strains of Bacillus anthracis and related bacteria.

3. An isolated nucleic acid comprising the nucleotide sequence of SEQ ID NO: 2.

4. The isolated nucleic acid as described in claim 3, wherein said nucleotide sequence is used for identifying strains of Bacillus anthracis and related bacteria.

5. An isolated nucleic acid comprising the nucleotide sequence of SEQ ID NO: 3.

6. The isolated nucleic acid as described in claim 5, wherein said nucleotide sequence is used for identifying strains of Bacillus anthracis and related bacteria.

7. An isolated nucleic acid comprising the nucleotide sequence of SEQ ID NO: 4.

8. The isolated nucleic acid as described in claim 7, wherein said nucleotide sequence is used for identifying strains of Bacillus anthracis and related bacteria.

9. An isolated nucleic acid comprising the nucleotide sequence of SEQ ID NO: 5.

10. The isolated nucleic acid as described in claim 9, wherein said nucleotide sequence is used for identifying strains of Bacillus anthracis and related bacteria.

11. An isolated nucleic acid comprising the nucleotide sequence of SEQ ID NO: 6.

12. The isolated nucleic acid as described in claim 11, wherein said nucleotide sequence is used for identifying strains of Bacillus anthracis and related bacteria.

13. An isolated nucleic acid useful for identifying strains of Bacillus anthracis comprising the nucleotide sequence of SEQ ID NO: 7.

14. The isolated nucleic acid as described in claim 13, wherein said nucleotide sequence is used for identifying strains of Bacillus anthracis and related bacteria.

15. An isolated nucleic acid comprising the nucleotide sequence of SEQ ID NO: 8.

16. The isolated nucleic acid as described in claim 15, wherein said nucleotide sequence is used for identifying strains of Bacillus anthracis and related bacteria.

17. An isolated nucleic acid comprising the nucleotide sequence of SEQ ID NO: 9.

18. The isolated nucleic acid as described in claim 17, wherein said nucleotide sequence is used for identifying strains of Bacillus anthracis and related bacteria.

19. An isolated nucleic acid comprising the nucleotide sequence of SEQ ID NO: 10.

20. The isolated nucleic acid as described in claim 19, wherein said nucleotide sequence is used for identifying strains of Bacillus anthracis and related bacteria.

21. An isolated nucleic acid comprising the nucleotide sequence of SEQ ID NO: 11.

22. The isolated nucleic acid as described in claim 21, wherein said nucleotide sequence is used for identifying strains of Bacillus anthracis and related bacteria.

23. An isolated nucleic acid comprising the nucleotide sequence of SEQ ID NO: 12.

24. The isolated nucleic acid as described in claim 23, wherein said nucleotide sequence is used for identifying strains of Bacillus anthracis and related bacteria.

25. An isolated nucleic acid comprising the nucleotide sequence of SEQ ID NO: 13.

26. The isolated nucleic acid as described in claim 25, wherein said nucleotide sequence is used for identifying strains of Bacillus anthracis and related bacteria.

27. An isolated nucleic acid comprising the nucleotide sequence of SEQ ID NO: 14.

28. The isolated nucleic acid as described in claim 27, wherein said nucleotide sequence is used for identifying strains of Bacillus anthracis and related bacteria.

29. An isolated nucleic acid comprising the nucleotide sequence of SEQ ID NO: 15.

30. The isolated nucleic acid as described in claim 29, wherein said nucleotide sequence is used for identifying strains of Bacillus anthracis and related bacteria.

31. An isolated nucleic acid comprising the nucleotide sequence of SEQ ID NO: 16.

32. The isolated nucleic acid as described in claim 31, wherein said nucleotide sequence is used for identifying strains of Bacillus anthracis and related bacteria.

33. An isolated nucleic acid comprising the nucleotide sequence of SEQ ID NO: 17.

34. The isolated nucleic acid as described in claim 33, wherein said nucleotide sequence is used for identifying strains of Bacillus anthracis and related bacteria.

35. An isolated nucleic acid comprising the nucleotide sequence of SEQ ID NO: 18.

36. The isolated nucleic acid as described in claim 35, wherein said nucleotide sequence is used for identifying strains of Bacillus anthracis and related bacteria.

37. An isolated nucleic acid comprising the nucleotide sequence of SEQ ID NO: 19.

38. The isolated nucleic acid as described in claim 37, wherein said nucleotide sequence is used for identifying strains of Bacillus anthracis and related bacteria.

39. An isolated nucleic acid comprising the nucleotide sequence of SEQ ID NO: 20.

40. The isolated nucleic acid as described in claim 39, wherein said nucleotide sequence is used for identifying strains of Bacillus anthracis and related bacteria.

41. An isolated nucleic acid comprising the nucleotide sequence of SEQ ID NO: 21.

42. The isolated nucleic acid as described in claim 41, wherein said nucleotide sequence is used for identifying strains of Bacillus anthracis and related bacteria.

43. An isolated nucleic acid comprising the nucleotide sequence of SEQ ID NO: 22.

44. The isolated nucleic acid as described in claim 43, wherein said nucleotide sequence is used for identifying strains of Bacillus anthracis and related bacteria.

45. An isolated nucleic acid comprising the nucleotide sequence selected from the group consisting of SEQ ID NO: 23 through SEQ ID NO: 77, and SEQ ID NO: 78.

46. The isolated nucleic acid as described in claim 45, wherein said nucleotide sequence is used for identifying strains of Bacillus anthracis and related bacteria.

47. An isolated nucleic acid comprising the nucleotide sequence selected from the group consisting of SEQ ID NO: 79 through SEQ ID NO: 105, and SEQ ID NO: 106.

48. The isolated nucleic acid as described in claim 47, wherein said nucleotide sequence is used for identifying strains of Bacillus anthracis and related bacteria.
